(19) United States
(12) Patent Application Publication
(10) Pub. No.: US 2025/0382670 A1
(43) Pub. Date: Dec. 18, 2025

(54) NOVEL COMPOSITIONS AND METHODS FOR CELL-FREE DNA DETECTION

(71) Applicants: JIANGSU CODE BIOMEDICAL TECHNOLOGY CO., LTD., Nanjing (CN); CODE BIOLOGICAL MEDICINE TECHNOLOGY INC., Ontario, CA (US)

(72) Inventors: Shiyang Pan, Nanjing (CN); Jian Xu, Nanjing (CN); Yuan Mu, Nanjing (CN); Yue Pan, Ontario, CA (US); Jianfeng Tao, Nanjing (CN)

(21) Appl. No.: 18/865,870

(22) PCT Filed: May 15, 2023
(86) PCT No.: PCT/CN2023/094285

§ 371 Date: Nov. 14, 2024

(30) Foreign Application Priority Data
May 14, 2022 (WO) PCT/CN2022/092895

(51) Int. Cl. C12Q 1/6883

(52) U.S. Cl. CPC C12Q 1/6883; C12Q 2600/118; C12Q 2600/156; C12Q 2600/166

(57) ABSTRACT

Provided herein are internal standard oligonucleotides, primers, probes and kits for the detection and quantification of cell-free DNA using multiplex quantitative real-time PCR.

### TECHNICAL FIELD

This disclosure relates to compositions and kits for detecting cell-free DNA and uses thereof. Specifically, primers and probes for multiplex quantitative real-time PCR and methods of detecting and quantifying circulating, cell-free DNA are provided.

### BACKGROUND

Since its discovery in human blood plasma about 70 years ago, circulating cell-free DNA (cfDNA) has become an attractive subject of research as noninvasive disease biomarker. The interest in clinical applications has gained an exponential increase, making it a popular and potential target in a wide range of research areas. cfDNA can be found in different body fluids, both in healthy and not healthy subjects. The recent and rapid development of new molecular techniques is promoting the study and the identification of cfDNA, holding the key to minimally invasive diagnostics, improving disease monitoring, clinical decision, and patients' outcome. cfDNA has already given a huge impact on prenatal medicine, and it could become, in the next future, the standard of care also in other fields, from oncology to transplant medicine and cardiovascular diseases (see, e.g., Ranucci et al., Methods Mol Biol. 2019; 1909:3-12). cfDNA can be detected in a variety of bodily fluids, such as blood (blood plasma or serum), urine, saliva, cerebrospinal fluid and synovial fluid. Therefore, the detection and quantification of cfDNA in bodily fluids can be impacted by a few factors such as the type of test samples, nucleic acid extraction methods, preservation methods, and the detection and quantification methods.

Different quantification methods can lead to large deviation of test results due to the relatively low cfDNA content in bodily fluids. Quantitative analyses using gene amplification means are used to solve this problem. Due to the loss of nucleic acid during the extraction process, quantitative result sometimes cannot reflect the actual cfDNA quantity in the bodily fluid. In addition, there are different degrees of DNA degradation (as high as 30%) either during or after purification or long-term preservation. These variations greatly affect the accuracy and interpretation of the final results.

Current methods for quantifying circulating cfDNA include quantitative, fluorescent PCR using an external standard and PicoGreen labels. The method uses a house-keeping gene as the quantitative standard and generate a standard curve using the known concentrations of the external standard. Because the external standard and the test sample are quantified in different containers, the variation between the quantification impacts the accuracy and stability of the results. In addition, because the method using PicoGreen fluorophores detects the cfDNA directly without amplification, the sensitivity of the quantification method is low and the variation between different tests is significant.

Severe acute respiratory syndrome coronavirus 2 (SARS-COV-2) is an enveloped, positive-strand RNA virus that causes the disease COVID-19 (Coronavirus Disease-2019). While coronaviruses typically cause relatively mild respiratory diseases, as of February 2021 COVID-19 is on course to kill 2.5 million people since its emergence in late 2019. While recent progress in vaccine development has been remarkable, the emergence of novel coronaviruses in human populations represents a continuing threat. SARS-COV-2 genome comprises the following open reading frames or ORFs, from its 5′ end to its 3′ end: ORF1ab corresponding to the non-structural proteins forming the transcription-replication complex, and ORF-S (the S gene), ORF-E (the E gene), ORF-M (the M gene) and ORF-N (the N gene) corresponding to the four major structural proteins, spike surface glycoprotein(S), envelope protein (E), membrane glycoprotein (M) and nucleocapsid protein (N). It also comprises several accessory proteins like ORFs interspersed among or overlapping the structural genes and corresponding to proteins of unknown function.

SARS-COV-2 RNA genome has a 5′ methylated cap and a 3′ polyadenylated tail, which allows the RNA to attach to the host cell's ribosome for translation. ORF1 b encodes a protein called RNA-dependent RNA polymerase (RdRp or nsp12), which allows the viral genome to be transcribed into new RNA copies using the host cell's machinery. The RdRp is the first protein to be made; once the gene encoding the RdRp is translated, translation is stopped by a stop codon. RNA-dependent RNA polymerase (RdRp, RDR) is an enzyme that catalyzes the replication of RNA from an RNA template. This is in contrast to a typical DNA-dependent RNA polymerase, which catalyzes the transcription of RNA from a DNA template. RdRP is an essential protein encoded in the genomes of all RNA-containing viruses with no DNA stage. It catalyzes synthesis of the RNA strand complementary to a given RNA template. The RNA replication process is a two-step mechanism. First, the initiation step of RNA synthesis begins at or near the 3′ end of the RNA template by means of a primer-independent (de novo), or a primer-dependent mechanism that utilizes a viral protein genome-linked (VPg) primer. The de novo initiation consists in the addition of a nucleoside triphosphate (NTP) to the 3′-OH of the first initiating NTP. During the following so-called elongation phase, this nucleotidyl transfer reaction is repeated with subsequent NTPs to generate the complementary RNA product. The protein nsp9 which is encoded by ORF1a may participate in viral replication by acting as Single-stranded RNA-binding protein. The protein nsp6, also encoded by ORF1a, plays a role in the initial induction of autophagosomes from host reticulum and later limits expansion of these phagosomes that are no longer able to deliver viral components to lysosomes.

Several variants of SARS-COV-2 carrying mutations on the Spike protein with a predicted impact on the epidemiology of the Covid-19 disease emerged since mid-2020 and are currently spreading worldwide. These variants of concern were first reported in the UK (lineage B.1.1.7; notable mutations N501Y, 69-70del, P681 H) and VOC-202102/02 (B.1.1.7 with E484K); South Africa (SA) (lineage B.1.351; notable mutations N501Y, E484K, K417N); Brazil (BR) (lineage P.1; notable mutations N501Y, E484K, K417T); UK and Nigeria (lineage B.1.525; notable mutations E484K, F888L, 69-70del) and are currently spreading to multiple countries around the world.

All these new variants of SARS-COV-2 are characterized by an enhanced human-to-human transmissibility in comparison to earlier variants of the virus. The UK, SA and BR variants all share the mutation N501Y in the receptor-binding region (RBD), predicted to increase the spike's binding affinity towards the human ACE2 receptor. Variants SA and BR share an additional mutation in this region (K417T/N) suspected to contribute to further binding affinity to hACE2. The UK variant carries another mutation outside the RBD (del69/70) with a predicted impact on transmissibility. Furthermore, the variants SA and BR share an additional mutation in the RBD (E484K) reported to enhance SARS-COV-2 ability to escape the immune response (both natural and vaccine induced). Monoclonal and serum-derived antibodies are reported to be from 10 to 60 time less effective in neutralizing virus bearing the E484K mutation. The distinct mutation L452R carried by the Californian variant was shown to enhance SARS-COV-2 immune evasion ability in previous studies. Some vaccines might see their efficacies reduced against these variants.

Consequently, these emerging variants of SARS-COV-2 are of concern due to their increased transmissibility (UK, BR, SA). Furthermore, the reduced sensitivity to neutralizing antibodies of the variants carrying the mutation E484K (SA, BR) may compromise vaccine effectiveness.

As a result, approaches that provide accurate and stable tests for cfDNA in bodily fluid samples and methods for predicting the severity of diseases such as SARS-COV-2 infection are needed in the art.

### SUMMARY

The disclosure relates to internal standard oligonucleotides, primers, probes and kits for the detection of cfDNA in bodily fluid samples using multiplex quantitative real-time PCR. The disclosure also relates to methods for detecting cfDNA and assessing severity of injuries or diagnosing diseases.

Accordingly, the current disclosure provides a double-stranded internal standard oligonucleotide for the detection of cell-free DNA in a biological sample, comprising a sequence that is at least 80% identical to the sequence of SEQ ID NO: 1.

In some embodiments, the oligonucleotide comprises a sequence consisting of SEQ ID NO: 1.

In some embodiments, the internal standard oligonucleotide has a length of about 100 bp to about 3000 bp.

In some embodiments, the internal standard oligonucleotide has a length of about 190 bp to about 200 bp.

The current disclosure also provides a method of generating an internal standard oligonucleotide described herein, comprising (a) providing a double-stranded oligonucleotide sequence that comprises a region of about 25-200 bp on a target human gene; (b) inserting the oligonucleotide into a recombination vector; (c) digesting the recombination vector of step (b) using one or more endonucleases, thereby obtaining a linear internal standard oligonucleotide.

In some embodiments, the recombination vector is a pMD20 vector.

In some embodiments, the one or more endonucleases comprises SmaI.

The current disclosure also provides an oligonucleotide comprising a sequence that is at least 90% identical to the full length of an oligonucleotide sequence selected from any one of SEQ ID NOs.: 2-6.

In some embodiments, the oligonucleotide is complementary and/or binds to human β-actin gene, and wherein the oligonucleotide comprises a sequence that is at least 90% identical to the full length of an oligonucleotide sequence of SEQ ID NO: 2 or 3.

In some embodiments, the oligonucleotide is complementary and/or binds to the sequence of SEQ ID NO: 1, and wherein the oligonucleotide comprises a sequence that is at least 90% identical to the full length of an oligonucleotide sequence of SEQ ID NO: 3 or 4.

In some embodiments, the oligonucleotide described herein comprises a sequence that is at least 90% identical to the full length of an oligonucleotide sequence of SEQ ID NO: 5 or 6, wherein the oligonucleotide has a 5′ terminus and 3′ terminus, and wherein the oligonucleotide is detectably labeled.

In some embodiments, the oligonucleotide comprises a sequence consisting of SEQ ID NO: 5.

In some embodiments, the oligonucleotide is detectably labeled with JOE at the 5′ terminus and/or wherein the oligonucleotide is detectably labeled with BHQ1 at the 3′ terminus.

In some embodiments, the oligonucleotide comprises a sequence consisting of SEQ ID NO: 6.

In some embodiments, the oligonucleotide is detectably labeled with FAM at the 5′ terminus and/or wherein the oligonucleotide is detectably labeled with BHQ1 at the 3′ terminus.

The current disclosure also provides a pharmaceutical composition comprising an effective amount of any of the oligonucleotides described herein, and a pharmaceutically acceptable carrier, diluent, or both.

The current disclosure also provides a method comprising contacting a biological sample with any of the oligonucleotides described herein.

In some embodiments, the method described herein further comprises detecting and quantifying a human β-actin gene in the biological sample.

In some embodiments, the method described herein further comprises quantifying cell-free DNA in the biological sample based on the quantification of the human β-actin gene.

The current disclosure also provides a method for detecting cell-free DNA in a biological sample, wherein said method comprises: (A) incubating the biological sample with: (1) a DNA polymerase and dNTP; (2) a forward primer for a human β-actin gene having a nucleotide sequence consisting of SEQ ID NO: 2; (3) a reverse primer for a human β-actin gene having a nucleotide sequence consisting of SEQ ID NO: 3; (4) a detectably labeled probe, wherein the probe comprises an oligonucleotide sequence that is able to specifically hybridize to an oligonucleotide sequence of human β-actin gene; wherein the incubation is in a reaction under conditions sufficient to permit the forward and reverse primers to mediate a polymerase chain reaction amplification of a region of the human β-actin gene to thereby produce an amplified human β-actin fragment, if said human β-actin in gene is present in said clinical sample; and (B) detecting the human β-actin gene; thereby detecting the presence of cell-free DNA in the biological sample.

In some embodiments, the method further comprises quantifying the human β-actin gene in the biological sample if said human β-actin gene is present in said clinical sample.

In some embodiments, the human β-actin probe is detectably labeled with JOE at the 5′ terminus and/or wherein the oligonucleotide is detectably labeled with BHQ1 at the 3′ terminus.

In some embodiments, the human β-actin probe comprises an oligonucleotide sequence of SEQ ID NO: 5.

In some embodiments, the human β-actin probe hybridizes to the amplified human β-actin fragments.

In some embodiments, the method described herein further comprises: (C) adding an amount of internal standard oligonucleotides having a sequence of SEQ ID NO: 1 to the biological sample; (D) incubating the biological sample in (C) with: (1) a DNA polymerase and dNTP; (2) a forward primer having a nucleotide sequence consisting of SEQ ID NO: 4; (3) a reverse primer having a nucleotide sequence consisting of SEQ ID NO: 3; (4) a detectably labeled internal standard probe, wherein the probe comprises an oligonucleotide sequence that is able to specifically hybridize to the internal standard oligonucleotide; wherein the incubation is in a reaction under conditions sufficient to permit the forward and reverse primers to mediate a polymerase chain reaction amplification of a region of the sequence of SEQ ID NO: 1 to thereby produce an amplified fragment of the region; (E) detecting the internal standard oligonucleotides.

In some embodiments, the internal standard probe is detectably labeled with FAM at the 5′ terminus and/or wherein the oligonucleotide is detectably labeled with BHQ1 at the 3′ terminus.

In some embodiments, the internal standard probe comprises an oligonucleotide sequence of SEQ ID NO: 6.

In some embodiments, the internal standard probe hybridizes to the region of SEQ ID NO: 1.

In some embodiments, the internal standard oligonucleotides 5×10⁴ copies in 5 μL are added to each 195 μL biological sample.

In some embodiments, the DNA polymerase has a 5′→3′ exonuclease activity that hydrolyzes the hybridized human β-actin probe or internal standard probe, to thereby separate the detectable labels on the probe and cause a signal to become detected.

In some embodiments, the hybridization of the probe to the amplified fragments separates the detectable labels on the probe and causes a signal to become detectable.

In some embodiments, the signal is a fluorescent signal.

In some embodiments, the probe is labeled with a fluorophore and a quencher of fluorescence of the fluorophore.

In some embodiments, the DNA polymerase is a Taq DNA polymerase.

The current disclosure also provides a method for quantifying cell-free DNA in a biological sample, wherein said method comprises: (A) incubating the biological sample with: (1) a DNA polymerase and dNTP; (2) a forward primer for a human β-actin gene having a nucleotide sequence consisting of SEQ ID NO: 2; (3) a reverse primer for a human β-actin gene having a nucleotide sequence consisting of SEQ ID NO: 3; (4) a detectably labeled probe, wherein the probe comprises an oligonucleotide sequence that is able to specifically hybridize to an oligonucleotide sequence of human β-actin gene; wherein the incubation is in a reaction under conditions sufficient to permit the forward and reverse primers to mediate a polymerase chain reaction amplification of a region of the human β-actin gene to thereby produce an amplified human β-actin fragment, if said human β-actin gene is present in said clinical sample; (B) adding an amount of internal standard oligonucleotides having a sequence of SEQ ID NO: 1 to the biological sample; (C) incubating the biological sample in (B) with: (1) a DNA polymerase and dNTP; (2) a forward primer having a nucleotide sequence consisting of SEQ ID NO: 4; (3) a reverse primer having a nucleotide sequence consisting of SEQ ID NO: 3; (4) a detectably labeled probe, wherein the probe comprises an oligonucleotide sequence that is able to specifically hybridize to the internal standard oligonucleotide; wherein the incubation is in a reaction under conditions sufficient to permit the forward and reverse primers to mediate a polymerase chain reaction amplification of a region of the sequence of SEQ ID NO: 1 to thereby produce an amplified fragment of the region; (D) detecting the internal standard oligonucleotide; (E) detecting and quantifying the human β-actin gene based on the detection of the internal standard oligonucleotide; thereby quantifying the cell-free DNA in the biological sample.

In some embodiments, the method described herein further comprises determining the amplification efficiency of the internal standard oligonucleotide and the human β-actin gene.

In some embodiments, the quantifying of the human β-actin gene is performed based on one or more of the parameters:
(1) The starting copy number of the internal standard oligonucleotide (S₀);
(2) The amplification efficiency of the human β-actin gene (E_{T});
(3) The amplification efficiency of the internal standard oligonucleotide (E_{S});
(4) The cycle threshold for the human β-actin gene (Ct,T); and
(5) The cycle threshold for the internal standard oligonucleotide (Ct,S).

In some embodiments, the quantifying of the human β-actin gene is performed according to the Formula (I)

$${T}_{0} = {S}_{0} \times \frac{{( 1 + {E}_{S} )}^{Ct , S}}{{( 1 + {E}_{T} )}^{Ct , T}} \qquad ( I )$$

The current disclosure also provides a kit, comprising:

(1) one or more internal standard oligonucleotide, wherein the one or more internal standard oligonucleotide comprises a sequence that is at least 90% identical to the sequence of SEQ ID NO: 1; (2) one or more oligonucleotide, wherein the one or more oligonucleotide comprises a sequence that is at least 90% identical to the full length of an oligonucleotide sequence selected from any one of SEQ ID NOs: 2-6; (3) a PCR buffer solution, a DNA polymerase, dNTP, and MgCl₂; (4) optionally instructions for performing any one of the methods described herein.

The current disclosure also provides an internal standard oligonucleotide, comprising: (a) an oligonucleotide sequence that is at least 80% identical to the corresponding region of a target human gene; (b) a forward primer binding site and a reverse primer binding site, wherein the length between the forward primer binding site and the reverse primer binding site is about 90 bp to about 200 bp.

In some embodiments, the reverse primer binding site is within the sequence that is at least 80% identical to the corresponding region of a human gene.

In some embodiments, the internal standard oligonucleotide has a length of about 100 bp to about 3000 bp.

In some embodiments, the target human gene is a human housekeeping gene.

In some embodiments, the housekeeping gene is a single-copy housekeeping gene.

In some embodiments, the housekeeping gene is selected from the group consisting of: human 18S rRNA (18S ribosomal RNA), human 28S rRNA (28S ribosomal RNA), human TUBA (α-tubulin), human ACTB (β-actin), human β2M ((2-microglobulin), human ALB (albumin), human RPL32 (ribosomal protein L32), human TBP (TATA sequence binding protein), human CYCC (cyclophilin C), human EF1A (elongation factor 1α), human GAPDH (glyceraldehyde-3-phosphate dehydrogenase), human HPRT (hypoxanthine phosphoribosyl transferase), and human RPII (RNA polymerase II).

In some embodiments, the internal standard oligonucleotide is double-stranded.

The current disclosure also provides a primer set for detecting cell-free DNA in a subject, comprising: (a) a forward primer and a reverse primer for amplifying a human gene in the biological sample; and (b) a forward primer and a reverse primer for amplifying an internal standard oligonucleotide; wherein the reverse primer for amplifying the human gene has a sequence that is at least 80% identical to the sequence of the reverse primer for amplifying the internal standard oligonucleotide.

In some embodiments, the reverse primer for amplifying the internal standard oligonucleotide has a sequence that is identical to the sequence of the reverse primer for amplifying the target gene.

The current disclosure also provides a primer set for detecting cell-free DNA in a subject, comprising: (a) a forward primer and a reverse primer for amplifying a human gene in the biological sample; and (b) a forward primer and a reverse primer for amplifying an internal standard oligonucleotide; wherein the forward primer for amplifying the internal standard oligonucleotide has a sequence that is at least 80% identical to the sequence of the forward primer for amplifying the human gene.

In some embodiments, the forward primer for amplifying the internal standard oligonucleotide has a sequence that is identical to the sequence of the forward primer for amplifying the target gene.

In some embodiments, the forward primer and the reverse primer for amplifying the human gene bind to regions on the human gene that are about 90 bp to about 200 bp apart. In some embodiments, the forward primer and the reverse primer for amplifying the internal standard oligonucleotide bind to regions on the internal standard oligonucleotide that are about 90 bp to about 200 bp apart.

In some embodiments, the forward and/or the reverse primer has a length of about 15 bp to about 30 bp.

The current disclosure also provides a kit, comprising:

(1) one or more internal standard oligonucleotide; (2) one or more primer set described herein; (3) a PCR buffer solution, a DNA polymerase, and dNTP; (4) optionally instructions for performing any one of the methods described herein.

The current disclosure also provides a method of generating an internal standard oligonucleotide for the detection of cell-free DNA, comprising (a) providing a double-stranded oligonucleotide sequence that comprises a region of about 25-150 bp on a target human gene; (b) inserting the oligonucleotide into a recombination vector; (c) digesting the recombination vector of step (b) using one or more endonucleases, thereby obtaining a linear internal standard oligonucleotide, wherein the internal standard oligonucleotide is about 100 to about 3000 bp in length.

In some embodiments, the biological sample is essentially free of cellular DNA.

In some embodiments, the method described herein further comprises removing cellular DNA from the biological sample.

In some embodiments, the cellular DNA is removed using centrifugation, microfluidic-based separation, columns or magnetic beads, or filtration-based separation. The current disclosure further provides a method of predicting the severity of an infection by SARS-COV-2, comprising: (A) obtaining a biological sample from a subject having a SARS-COV-2 infection; (B) quantifying cell-free DNA (cfDNA) in the biological sample; (C) predicting the severity based on the quantification of the cfDNA, wherein a cfDNA concentration above a cut-off value indicates deterioration of the SARS-COV-2 infection.

In some embodiments, the quantification of the cfDNA comprises quantifying a housekeeping gene in the biological sample.

In some embodiments, the housekeeping gene is a human β-actin gene.

In some embodiments, the quantification of the cfDNA comprises: incubating the biological sample with: (1) a DNA polymerase and dNTP; (2) a forward primer for a human β-actin gene; (3) a reverse primer for a human β-actin gene; (4) a detectably labeled probe, wherein the probe comprises an oligonucleotide sequence that is able to specifically hybridize to an oligonucleotide sequence of human β-actin gene; wherein the incubation is in a reaction under conditions sufficient to permit the forward and reverse primers to mediate a polymerase chain reaction amplification of a region of the human β-actin gene to thereby produce an amplified human β-actin fragment, if said human β-actin gene is present in said clinical sample; adding an amount of an internal standard oligonucleotides to the biological sample; and incubating the biological sample with: (1) a DNA polymerase and dNTP; (2) a forward primer for the internal standard oligonucleotide; (3) a reverse primer for the internal standard oligonucleotide; (4) a detectably labeled probe, wherein the probe comprises an oligonucleotide sequence that is able to specifically hybridize to the internal standard oligonucleotide; wherein the incubation is in a reaction under conditions sufficient to permit the forward and reverse primers to mediate a polymerase chain reaction amplification of a region of the internal standard oligonucleotide to thereby produce an amplified fragment of the amplified region; detecting the internal standard oligonucleotide; and detecting and quantifying the human β-actin gene based on the detection of the internal standard oligonucleotide.

In some embodiments, the cut-off value is about 90 ng/ml to about 350 ng/ml. In some embodiments, the cut-off value is about 169.3 ng/mL.

In some embodiments, the prediction of the deterioration of the SARS-COV-2 infection has a sensitivity of at least 80%, 85%, 90%, 95%, 99% or higher.

In some embodiments, the prediction of the deterioration of the SARS-COV-2 infection has a sensitivity of at least 85%.

In some embodiments, the prediction of the deterioration of the SARS-COV-2 infection has a specificity of at least 80%, 85%, 90%, 95%, 99% or higher.

In some embodiments, the prediction of the deterioration of the SARS-COV-2 infection has a specificity of at least 86%.

In some embodiments, the prediction of the severity of the SARS-COV-2 infection is based on one or more further indicators selected from demographic variables, clinical signs and symptoms, imaging results, laboratory findings, and medical history.

In some embodiments, the clinical signs and symptoms are selected from body temperature, systolic blood pressure, diastolic blood pressure, heart rate, respiratory rate, vasoactive agents administration, sedative agents administration, analgesic agents administration and unconsciousness.

In some embodiments, the imaging results are selected from abnormality of chest radiography and CT imaging.

In some embodiments, the laboratory findings are selected from partial arterial oxygen pressure, oxygen saturation, white blood cell counts and differentiation, neutrophil to lymphocyte ratio (NLR), platelet counts, hematocrit, serum sodium and potassium, pH, total bilirubin, creatinine, and D-dimer levels.

In some embodiments, the medical history is selected from past operation, chronic obstructive pulmonary disease, liver cirrhosis, renal dialysis, immunodeficiency disease, cancer, chemotherapy, radiation, long term and high dose steroids.

In some embodiments, the prediction of the severity of the SARS-COV-2 infection further comprises calculating the Acute Physiology and Chronic Health Evaluation (APACHE II) and/or Sequential Organ Failure Assessment (SOFA) scores on the worst value for one or more physiological variables.

In some embodiments, the calculation of the APACHE II and SOFA scores is performed within 24 hours of the time point when the biological sample is collected.

In some embodiments, the forward primer for the human β-actin gene has a nucleotide sequence that is at least 80% identical to SEQ ID NO: 2, and the reverse primer for the human β-actin gene has a nucleotide sequence that is at least 80% identical to SEQ ID NO: 3.

In some embodiments, the detectably labeled probe for human β-actin gene has a sequence that is at least 80% identical to SEQ ID NO: 5.

In some embodiments, the forward primer for the internal standard oligonucleotide has a nucleotide sequence that is at least 80% identical to SEQ ID NO: 4, and the reverse primer for the internal standard oligonucleotide has a nucleotide sequence that is at least 80% identical to SEQ ID NO: 3.

In some embodiments, the detectably labeled probe for the internal standard oligonucleotide has a sequence that is at least 80% identical to SEQ ID NO: 6.

In some embodiments, the internal standard oligonucleotide has a sequence that is at least 80% identical to SEQ ID NO: 1.

In some embodiments, the internal standard oligonucleotide consists of a sequence of SEQ ID NO: 1.

In some embodiments, the severe status of the SARS-COV-2 infection corresponds to an APACHE II score greater than 15 (>15).

In some embodiments, the severe status of the SARS-COV-2 infection corresponds to SOFA score greater than or equal to 2 (>2).

In some embodiments, a cfDNA concentration below the cut-off value indicates a non-severe status of the SARS-COV-2 infection.

In some embodiments, the non-severe status of the SARS-COV-2 infection corresponds to an APACHE II score less than or equal to 15 (≤15).

In some embodiments, the non-severe status of the SARS-COV-2 infection corresponds to a SOFA score less than 2 (<2).

In some embodiments, the method described herein further comprises determining a treatment plan for the SARS-COV-2 infection.

In some embodiments, the treatment plan for the deterioration of the SARS-COV-2 infection is selected from ICU admission, intratracheal intubation, hormone therapy, and ECMO treatment.

In some embodiments, the treatment plan for the non-severe status of the SARS-COV-2 infection is selected from reducing the dosage of current administration of therapeutic agents, release from ICU, discharge from hospitalization.

In some embodiments, the method described herein further comprises determining a baseline quantity of cfDNA in the biological sample.

In some embodiments, the method described herein further comprises collecting one or more additional biological samples to determine the cfDNA level at one or more additional time points.

In some embodiments, the method further comprises monitoring the levels of cfDNA from different time points over a certain time period.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

The following drawings form part of the present specification and are included to further demonstrate certain aspects of the invention. The invention may be better understood by reference to one or more of these drawings in combination with the detailed description of specific embodiments presented herein:

FIG. 1 shows the sources of cfDNA in human biological samples.

FIG. 2 is a schematic illustration of an internal standard oligonucleotide described herein. a) is the full-length region of the internal standard oligonucleotide, b) is the amplifiable region of the internal standard oligonucleotide, and c) is the reverse primer binding region (e.g., the same sequence as the reverse primer binding region of the human β-actin gene).

FIG. 3 depicts the amplification efficiency for the human β-actin gene and the internal standard oligonucleotide. a) is a graph showing the amplification curves of the internal standard oligonucleotide using serial dilutions, b) is the linear regression representation of the amplification of the internal standard oligonucleotide, c) is a graph showing the amplification curves of the human β-actin gene using serial dilutions, and d) is the linear regression representation of the amplification of the human β-actin gene.

FIG. 4 shows the linear range of the detection of cfDNA using a method described herein.

FIGS. 5A-5B depict the workflow of detecting and quantifying cfDNA using a method described herein.

FIG. 6 shows the distribution of cfDNA concentrations from 213 samples using a method described herein.

FIG. 7 shows the distribution of cfDNA concentrations grouped by gender using a method described herein.

FIG. 8 shows the nucleic acid sequence of SEQ ID NO: 1.

FIG. 9 describes the therapeutic management of adults hospitalized for COVID-19 based on disease severity. Key: ECMO=extracorporeal membrane oxygenation; ED=emergency department; Hgb=hemoglobin; ICU=intensive care unit; IL=interleukin; IV=intravenous; JAK=Janus kinase; LMWH=low-molecular-weight heparin; mAb=monoclonal antibody; MV=mechanical ventilation; NIV=noninvasive ventilation; the Panel=the COVID-19 Treatment Guidelines Panel; UFH=unfractionated heparin; ULN=upper limit of normal; VTE=venous thromboembolism.

FIGS. 10A-10B show the dynamics of plasma DNA and severity scores in monitoring hospitalized patients with COVID-19. Timeline charts illustrate the APACHE II score (solid column), SOFA score (hollowed column) and plasma DNA (area in purple) in all the 17 patients.

FIGS. 11A-E show the development and performance of the prediction model. FIG. 11A is a nomogram for the prediction of COVID-19 deterioration. FIGS. 11B-C show the calibration curves of the nomogram in developing (B) and validating subset (C), respectively.

FIGS. 11D-E show the ROC curves of the prediction model in the developing (D) and validating subset (E), respectively.

FIGS. 12A-B show the prediction performance of a concise model using only plasma DNA. FIG. 12A shows the ROC curve of the model using only plasma DNA in the developing subset. FIG. 12B shows the decision curve analysis for models using two-index (plasma DNA and neutrophil count) or a concise version (only plasma DNA).

FIG. 13 shows the distribution of D-dimer between raw data and imputed data. Fifteen missing values of D-dimer were imputed (in red) using multiple imputation of predictive mean matching (PMM) from the whole dataset with nearest neighbors of 3 for this continuous variable. The introduced influence from imputation was evaluated through distribution comparison to the raw data (in blue), which had no significant differences (P=0.685).

FIGS. 14A-B show an example study workflow. The flow diagrams show the study population enrollment and observation period.

FIG. 15 shows the receiver operating characteristic (ROC) analysis for discrimination of severe status by plasma DNA and APACHE II score.

FIGS. 16A-B show the Spearman's correlation of plasma DNA to APACHE II and SOFA scores under severe (A) and non-severe (B) status.

FIG. 17 is a diagram showing the monitoring of plasma DNA in a case study (Case #1).

FIG. 18 is a diagram showing the monitoring of plasma DNA in a case study (Case #3).

FIG. 19 is a diagram showing the monitoring of plasma DNA in a case study (Case #4).

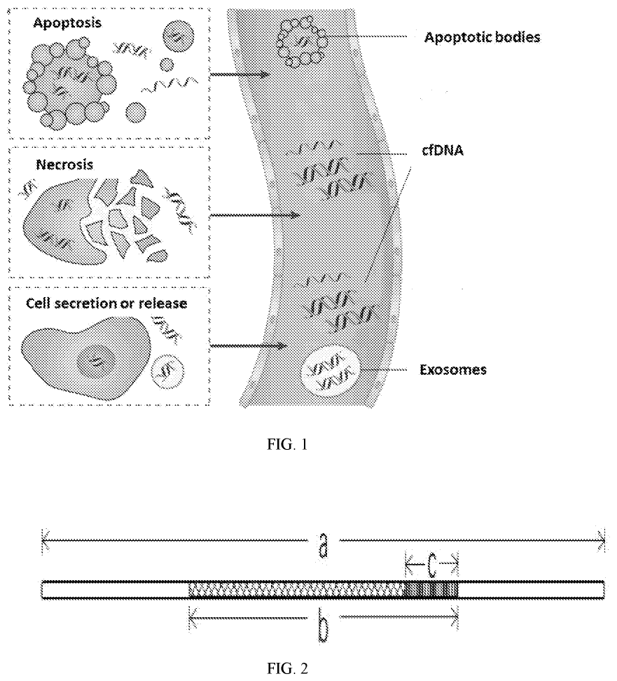

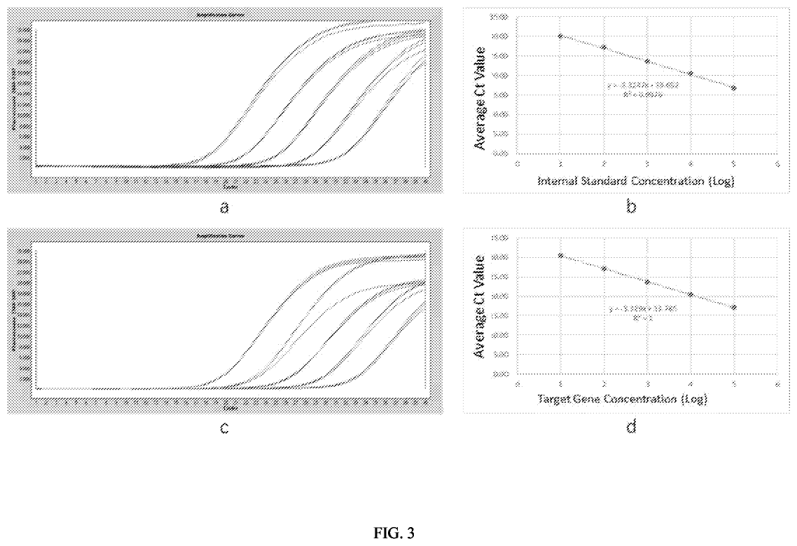

### DETAILED DESCRIPTION

The disclosure relates to compositions and methods for the detection and quantification of cell-free DNA (cfDNA). Specifically, internal standard oligonucleotides, probes and primers, and kits for performing multiplex quantitative real-time PCR are provided. The kit provided herein comprises multiplex quantitative real-time PCR primers and probes for detecting the human β-actin gene and the internal standard oligonucleotide.

As used herein, the singular forms “a”, “an”, and “the” include plural reference unless the context clearly dictates otherwise.

The term “about” is used herein to mean within the typical ranges of tolerances in the art. For example, “about” can be understood as about 2 standard deviations from the mean. In certain embodiments, about means ±10%. In certain embodiments, about means ±5%. When about is present before a series of numbers or a range, it is understood that “about” can modify each of the numbers in the series or range.

### Cell-Free DNA

Cell-free DNA (cfDNA) is the extracellular DNA existing in bodily fluids such as plasma. The cfDNA level in healthy people is normally stable and remains at low levels. The cfDNA level in healthy people is less or equal to 30 ng/mL. In situations such as infection, trauma, sepsis, acute respiratory distress syndrome (ARDS) and respiratory failure causing an abnormal amount of dead cells and released cfDNA into circulation, significantly increasing its level in plasma. As such, cfDNA can act as an indicator directly reflecting the degree of tissue or organ damage/failure caused by the complications which occurred in some COVID-19 patients.

The presence of cell-free DNA (cfDNA) in blood plasma was discovered in 1948 by Mandel and Metais (see, e.g., Mandel P, Metais P, Les acides nucléques du plasma sanguin chez l′homme, C R Seances Soc Biol Fil 1948; 142:241-3). Seventeen years later, in 1965, Bendich and colleagues hypothesized, that cancer-derived cfDNA could be involved in metastasis (Bendich A, Wilczok T, Borenfreund E. Circulating DNA as a possible factor in oncogenesis. Science 1965; 148:374-6). However, it took another year to discover the first link to disease. In 1966, Tan and colleagues observed high levels of circulating cell-free DNA (cfDNA) in the blood of systemic lupus erythematosus patients (Tan E M, Schur P H, Carr R I, Kunkel H G. Deoxybonucleic acid (DNA) and antibodies to DNA in the serum of patients with systemic lupus erythematosus. J Clin Invest 1966; 45:1732-40). Eleven years later, in 1977, Leon and colleagues used radioimmunochemistry to demonstrate that for at least half of cancer patients the level of cfDNA in their blood was significantly higher than in normal control subjects (Leon S A, Shapiro B, Sklaroff D M, Yaros M J. Free DNA in the serum of cancer patients and the effect of therapy. Cancer Res 1977; 37:646-50). The authors noted that patients with metastatic cancer had significantly higher cfDNA levels in blood. Because of technological limitations, it took another 12 years for the first experimental evidence to support that cfDNA in cancer patients does indeed contain tumor DNA based on temperature stability measurements (Stroun M, Anker P, Maurice P, Lyautey J, Lederrey C, Beljanski M. Neoplastic characteristics of the DNA found in the plasma of cancer patients. Oncology 1989; 46:318-22).

Recent studies linked cfDNA levels to outcomes in severe injury such as blunt trauma and burns (see, e.g., Butt A N, Swaminathan R. Overview of circulating nucleic acids in plasma/serum. Ann N Y Acad Sci 2008; 1137:236-42). Quantitative analysis of cfDNA and plasma APC/RASSFIA methylation provide a real-time indicator for monitoring efficiency and toxicity of chemotherapy (see, e.g., Wang et al. Clinical Epigenetics 2015; 7:119, the entire content of which is incorporated herein by reference). cfDNA levels correlated with the length of hospital stay, burn surface area, the number of operations needed for scalds (though not for the flash/flame burns). Plasma cfDNA levels also correlated with the need for patient ventilation in intensive care units (ICU). In line with these findings, cfDNA levels in blood turned out to be higher and have certain predictive value for sepsis and septic shock, aseptic inflammation, myocardial infarction, stroke including patients with negative neuroimaging results, where cfDNA concentrations seem to predict poststroke morbidity and mortality in patients with negative neuroimaging, and sickle cell disease. In short, cfDNA concentration is elevated in conditions that involve increased rates of cell death (apoptosis or necrosis).

Multiple properties of cfDNA suggest cell death as its major origin. Importantly, cfDNA is double-stranded and highly fragmented, with most molecules being approximately 200 bp (e.g., with a distribution of about 160 bp to about 240 bp).

Timely evaluation of disease severity and patient triage is crucial in providing optimized supporting care and appropriate interventions during hospitalization to improve the cure rate and reduce mortality; however, currently there is a paucity of accurate and objective laboratory indicators available to clinicians.

Provided herein are internal standard oligonucleotides for the detection and quantification of cfDNA in biological samples, e.g., bodily fluids. In some embodiments, the quantity of the cfDNA is measured using a quantity of a representative gene. In some embodiments, the representative gene is a house-keeping gene. In some embodiments, the house-keeping gene is a human β-actin gene.

### Internal Standard Oligonucleotide

Provided herein are internal standard oligonucleotides for the detection of cfDNA in a biological sample.

The term “oligonucleotide” is used herein to refer to a relatively short nucleic acid fragment or sequence. It can comprise DNA, RNA, or a hybrid thereof, or chemically modified analog or derivatives thereof. They can be single-stranded or double-stranded having two complementing strands which can be separated by denaturation. In some embodiments, the internal standard oligonucleotide is a double-stranded DNA oligonucleotide.

The disclosure also relates to an internal standard oligonucleotide including: (a) a forward primer binding site and a reverse primer binding site; and (b) an amplifiable sequence between the forward primer binding site and the reverse primer binding site, wherein the amplifiable sequence has a length that is substantially similar to an amplified sequence of a human gene, e.g., about 95% to about 105% of the length of the amplified sequence, about 90% to about 110% of the length of the amplified sequence, about 80% to about 120% of the length of the amplified sequence, or about 70% to about 130% of the length of the amplified sequence.

In some embodiments, the GC contents in the amplifiable sequence is similar to the amplified sequence of the human gene.

The disclosure also relates to an internal standard oligonucleotide including: (a) an oligonucleotide sequence that is at least 70%, at least 75%, at least 80%, at least 85%, at least 90%, at least 95%, at least 99%, or 100% identical to the corresponding region of a human gene; (b) a forward primer binding site and a reverse primer binding site.

In some embodiments, provided herein is an internal standard oligonucleotide sequence, comprising: a forward primer binding sequence and a reverse primer binding sequence, wherein the length between the forward primer binding sequence and the reverse primer binding sequence is about 90 bp to about 200 bp, wherein the reverse primer binding sequence is identical to the reverse primer binding sequence on the target gene sequence.

In some embodiments, provided herein is an internal standard oligonucleotide sequence, comprising: a forward primer binding sequence and a reverse primer binding sequence, wherein the length between the forward primer binding sequence and the reverse primer binding sequence is about 90 bp to about 200 bp, wherein the forward primer binding sequence is identical to the forward primer binding sequence on the target gene sequence.

In some embodiments, the target human gene is a human β-actin gene. In some embodiments, the human gene is a human house-keeping gene. In some embodiments, the housekeeping gene is a single-copy housekeeping gene. In some embodiments, the housekeeping gene is selected from the group consisting of: human 18S rRNA (18S ribosomal RNA), human 28S rRNA (28S ribosomal RNA), human TUBA (α-tubulin), human ACTB (β-actin), human β2M (β2-microglobulin), human ALB (albumin), human RPL32 (ribosomal protein L32), human TBP (TATA sequence binding protein), human CYCC (cyclophilin C), human EF1A (elongation factor 1α), human GAPDH (glyceraldehyde-3-phosphate dehydrogenase), human HPRT (hypoxanthine phosphoribosyl transferase), and human RPII (RNA polymerase II).

In some embodiments, the internal standard oligonucleotide is double-stranded. In some embodiments, the internal standard oligonucleotide is partially double-stranded and partially single-stranded. In some embodiments, the internal standard oligonucleotide is single-stranded.

The internal standard oligonucleotides can have a length of from about 100 nucleotides to about 3000 nucleotides. In some embodiments, the internal standard oligonucleotide is about 100 bp to about 3000 bp in length. In some embodiments, the internal standard oligonucleotide is about 100 bp to about 2000 bp in length. In some embodiments, the internal standard oligonucleotide is about 100 bp to about 1000 bp in length. In some embodiments, the internal standard oligonucleotide is about 100 bp to about 500 bp in length. In some embodiments, the internal standard oligonucleotide is about 100 bp to about 200 bp in length. In some embodiments, the internal standard oligonucleotide is about 150 bp to about 200 bp in length. In some embodiments, the internal standard oligonucleotide is about 180 bp to about 210 bp in length. In some embodiments, the internal standard oligonucleotide is about 190 bp to about 200 bp in length. In some embodiments, the internal standard oligonucleotide is about 200 bp in length. The internal standard oligonucleotides can be labeled with detectable markers or modified using conventional manners for various molecular biological applications.

The internal standard oligonucleotide can include a sequence that is at least 70%, 75%, 80%, 85%, 90%, 95% or 100% identical to a sequence of a target human gene, or a portion thereof. In some embodiments, the internal standard oligonucleotide includes a sequence that is at least 70%, 75%, 80%, 85%, 90%, 95% or 100% identical to a target gene, e.g., a human β-actin gene, or a portion thereof. In some embodiments, the internal standard oligonucleotide includes a sequence of about 10 bp to about 300 bp, about 20 bp to about 300 bp, about 30 bp to about 300 bp, about 40 bp to about 300 bp, about 50 bp to about 300 bp, about 60 bp to about 300 bp, about 70 bp to about 300 bp, about 80 bp to about 300 bp, about 90 bp to about 300 bp, about 100 bp to about 300 bp, about 150 bp to about 300 bp, about 200 bp to about 300 bp, about 250 bp to about 300 bp, about 10 bp to about 200 bp, about 25 bp to about 200 bp, about 50 bp to about 200 bp, about 100 bp to about 200 bp, about 150 bp to about 200 bp, about 10 bp to about 100 bp, about 25 bp to about 100 bp, or about 50 bp to about 100 bp that is identical to a target gene, e.g., a human β-actin gene. In some embodiments, the internal standard oligonucleotide includes a sequence of about 25 bp to about 200 bp that is identical to a portion of a target gene, e.g., a human β-actin gene.

The internal standard oligonucleotides described herein are designed to be used to generate amplification products (e.g., PCR products) that have similar length to the amplification products generated based on the cfDNA in the biological sample. The similar lengths of the amplification product contribute to the improved amplification efficiency and more accurate quantification of the cfDNA. In some embodiments, the length between the forward primer binding sequence and the reverse primer binding sequence on the internal standard oligonucleotide is substantially identical to the length of the amplified target gene sequence (e.g., with no more than 5, 10, 15, or 20 bp difference).

In some embodiments, the overlapping region between the internal standard oligonucleotide and the human gene is adjusted based on the selected target human gene. For example, internal standard oligonucleotides that have an overlapping region with human β-actin gene.

An example of the nucleic acid sequence of the human β-actin gene is NCBI Reference Sequence: NG_007992.1 (SEQ ID NO: 7)

In some embodiments, the length between the forward primer binding site and the reverse primer binding site is about 25 bp to about 300 bp, about 25 bp to about 250 bp, about 25 bp to about 200 bp, about 25 bp to about 150 bp, about 25 bp to about 100 bp, about 25 bp to about 50 bp, about 50 bp to about 300 bp, about 50 bp to about 250 bp, about 50 bp to about 200 bp, about 50 bp to about 150 bp, about 50 bp to about 100 bp, about 100 bp to about 300 bp, about 100 bp to about 250 bp, about 100 bp to about 200 bp, about 100 bp to about 150 bp, about 150 bp to about 300 bp, about 150 bp to about 250 bp, or about 150 bp to about 200 bp. In some embodiments, the length between the forward primer binding site and the reverse primer binding site is about 90 bp to about 200 bp.

In some embodiments, the reverse primer binding site is within the sequence that is at least 60%, at least 65%, at least 70%, at least 75%, at least 80%, at least 85%, at least 90%, at least 95%, at least 99 or 100% identical to the corresponding region of a human gene.

In some embodiments, an amplification product (e.g., PCR product) is generated using the forward and reverse primers and the internal standard oligonucleotide as the template. In some embodiments, the amplification product has a length of about 25 bp to about 300 bp, about 25 bp to about 250 bp, about 25 bp to about 200 bp, about 25 bp to about 150 bp, about 25 bp to about 100 bp, about 25 bp to about 50 bp, about 50 bp to about 300 bp, about 50 bp to about 250 bp, about 50 bp to about 200 bp, about 50 bp to about 150 bp, about 50 bp to about 100 bp, about 100 bp to about 300 bp, about 100 bp to about 250 bp, about 100 bp to about 200 bp, about 100 bp to about 150 bp, about 150 bp to about 300 bp, about 150 bp to about 250 bp, or about 150 bp to about 200 bp. In some embodiments, the length between the forward primer binding site and the reverse primer binding site is about 90 bp to about 200 bp.

In some embodiments, the internal standard oligonucleotide has a sequence that is at least 70%, 75%, 80%, 85%, 90%, 95%, or 100% identical to the sequence of SEQ ID NO: 1.

SEQ ID NO: 1 (5′ to 3′):
5′ ccggctcgtatgttgtgtggaattgtgagcggataacaatttca
cacaggaaacagctatgaccatgattacgaattcgagctcggtaccc
ggggatcctctagagattacgggagcggttggtggtggaaatcgtgc
gtgacattaagaatcgtcgacctgcaggcatgcaagcttggcactgg
ccgtcgttttacaac 3′

The sequence of SEQ ID NO: 1 is also shown in FIG. 8.

The disclosure also relates to a method of generating an internal standard oligonucleotide described herein, including (a) providing a double-stranded oligonucleotide sequence that comprises a region of about 25-200 bp on a target human gene; (b) inserting the oligonucleotide into a recombination vector; (c) digesting the recombination vector of step (b) using one or more endonucleases, thereby obtaining a linear internal standard oligonucleotide.

In some embodiments, the double-stranded oligonucleotide sequence in step (a) is obtained from direct chemical synthesis. In some embodiments, the double-stranded oligonucleotide sequence in step (a) is obtained and/or amplified from a human gene, e.g., through molecular cloning (e.g., through enzyme digestion, insertion and ligation into vectors). In some embodiments, the full-length internal standard oligonucleotide is obtained from chemical synthesis.

In some embodiments, the target human gene is a human β-actin gene. In some embodiments, the human gene is a human house-keeping gene. In some embodiments, the housekeeping gene is a single-copy housekeeping gene. In some embodiments, the housekeeping gene is selected from the group consisting of: human 18S rRNA (18S ribosomal RNA), human 28S rRNA (28S ribosomal RNA), human TUBA (α-tubulin), human ACTB (β-actin), human β2M (B2-microglobulin), human ALB (albumin), human RPL32 (ribosomal protein L32), human TBP (TATA sequence binding protein), human CYCC (cyclophilin C), human EF1A (elongation factor 1α), human GAPDH (glyceraldehyde-3-phosphate dehydrogenase), human HPRT (hypoxanthine phosphoribosyl transferase), and human RPII (RNA polymerase II).

In some embodiments, the internal standard oligonucleotide is double-stranded. In some embodiments, the internal standard oligonucleotide is partially double-stranded and partially single-stranded. In some embodiments, the internal standard oligonucleotide is single-stranded.

The vector can be any suitable vectors known in the art. For example, suitable vectors include but are not limited to pUC12, pUC13, pUC18, pUC19, pUC57, pUC120, pMD2.G, pMD18-T, pMDIAI, and pMDISI. In some embodiments, the vector is a pMD20 vector.

Any suitable restriction endonucleases can be used in the methods described herein. Examples of restriction endonucleases include, but are not limited to, HhaI, HindIII, NotI, BbvCI, BglI, EcoRI, FokI, AlwI, SmaI, SphI, Sse8387 I, PstI, Hin II, AccI, SalI, EcoRV, XbaI, BamHI, XmaI, KpnI, and SacI. In some embodiments, the restriction endonuclease used in the method described herein is SmaI.

### Primers and Probes

Provided herein are oligonucleotides (e.g., primers and probes) for the detection of cfDNA in a biological sample. Specifically, provided herein are primers and probes for the detection of human β-actin in the biological sample and primers and probes for the detection of the internal standard oligonucleotide described herein (e.g., to generate a standard curve for the quantification of the human β-actin gene).

The disclosure provides a primer set for detecting cell-free DNA in a subject, comprising: (a) a forward primer and a reverse primer for amplifying a human gene in the biological sample; wherein (b) a forward primer and a reverse primer for amplifying an internal standard oligonucleotide; wherein the reverse primer for amplifying the human gene has an sequence that is at least 60%, at least 65%, at least 70%, at least 75%, at least 80%, at least 85%, at least 90%, at least 95%, at least 99%, or 100% identical to the sequence of the reverse primer for amplifying the internal standard oligonucleotide.

In some embodiments, the human gene is a human β-actin gene. In some embodiments, the human gene is a human house-keeping gene. In some embodiments, the housekeeping gene is a single-copy housekeeping gene. In some embodiments, the housekeeping gene is selected from the group consisting of: human 18S rRNA (18S ribosomal RNA), human 28S rRNA (28S ribosomal RNA), human TUBA (α-tubulin), human ACTB (β-actin), human β2M (B2-microglobulin), human ALB (albumin), human RPL32 (ribosomal protein L32), human TBP (TATA sequence binding protein), human CYCC (cyclophilin C), human EF1A (elongation factor 1α), human GAPDH (glyceraldehyde-3-phosphate dehydrogenase), human HPRT (hypoxanthine phosphoribosyl transferase), and human RPII (RNA polymerase II).

The internal standard oligonucleotide can be any one of the internal standard oligonucleotide described herein.

In some embodiments, the reverse primer for amplifying the internal standard oligonucleotide has a sequence that is identical to the sequence of the reverse primer for amplifying the target human gene.

In some embodiments, the forward primer and the reverse primer for amplifying the human gene bind to regions on the human gene that are about 25 bp to about 300 bp, about 25 bp to about 250 bp, about 25 bp to about 200 bp, about 25 bp to about 150 bp, about 25 bp to about 100 bp, about 25 bp to about 50 bp, about 50 bp to about 300 bp, about 50 bp to about 250 bp, about 50 bp to about 200 bp, about 50 bp to about 150 bp, about 50 bp to about 100 bp, about 100 bp to about 300 bp, about 100 bp to about 250 bp, about 100 bp to about 200 bp, about 100 bp to about 150 bp, about 150 bp to about 300 bp, about 150 bp to about 250 bp, or about 150 bp to about 200 bp apart. In some embodiments, the forward primer and the reverse primer for amplifying the human gene bind to regions on the human gene that are about 90 bp to about 200 bp apart.

In some embodiments, the forward primer and the reverse primer for amplifying the internal standard oligonucleotide bind to regions on the human gene that are about 25 bp to about 300 bp, about 25 bp to about 250 bp, about 25 bp to about 200 bp, about 25 bp to about 150 bp, about 25 bp to about 100 bp, about 25 bp to about 50 bp, about 50 bp to about 300 bp, about 50 bp to about 250 bp, about 50 bp to about 200 bp, about 50 bp to about 150 bp, about 50 bp to about 100 bp, about 100 bp to about 300 bp, about 100 bp to about 250 bp, about 100 bp to about 200 bp, about 100 bp to about 150 bp, about 150 bp to about 300 bp, about 150 bp to about 250 bp, or about 150 bp to about 200 bp apart. In some embodiments, the forward primer and the reverse primer for amplifying the internal standard oligonucleotide bind to regions on the human gene that are about 90 bp to about 200 bp apart. The forward and/or the reverse primer can be of any suitable length. In some embodiments, the forward and/or the reverse primer has a length of about 10 bp to about 40 bp. In some embodiments, the forward and/or the reverse primer has a length of about 15 bp to about 30 bp. In some embodiments, the reverse primer for the internal standard oligonucleotide and the target human gene are identical. In some embodiments, the forward primer for the internal standard oligonucleotide and the target human gene are identical.

The disclosure also provides sets of probes and primers for detecting and quantifying the human β-actin gene, e.g., using multiplex quantitative real-time PCR. In some embodiments, the forward primer for amplifying the human β-actin gene includes an oligonucleotide sequence of SEQ ID NO: 2; the reverse primer for amplifying the human β-actin gene includes an oligonucleotide sequence of SEQ ID NO: 3; and the probe for detecting the human β-actin gene includes an oligonucleotide sequence of SEQ ID NO: 5. In some embodiments, the forward primer for amplifying the internal standard oligonucleotide includes an oligonucleotide sequence of SEQ ID NO: 4; the reverse primer for amplifying the internal standard oligonucleotide includes an oligonucleotide sequence of SEQ ID NO: 3; and the probe for detecting the internal standard oligonucleotide includes an oligonucleotide sequence of SEQ ID NO: 6.

The oligonucleotide sequences of SEQ ID NOs: 1-6 are shown in Table 1. In some embodiments, the oligonucleotide described herein includes a sequence that is at least 70%, 75%, 80%, 85%, 90%, 95%, 96%, 97%, 98%, 99%, or 100% identical to the full length of an oligonucleotide sequence selected from any one of SEQ ID NO: 1. In some embodiments, the oligonucleotide described herein includes a sequence that is at least 70%, 75%, 80%, 85%, 90%, 95%, 96%, 97%, 98%, 99%, or 100% identical to the full length of an oligonucleotide sequence selected from any one of SEQ ID NO: 2. In some embodiments, the oligonucleotide described herein includes a sequence that is at least 70%, 75%, 80%, 85%, 90%, 95%, 96%, 97%, 98%, 99%, or 100% identical to the full length of an oligonucleotide sequence selected from any one of SEQ ID NO: 3. In some embodiments, the oligonucleotide described herein includes a sequence that is at least 70%, 75%, 80%, 85%, 90%, 95%, 96%, 97%, 98%, 99%, or 100% identical to the full length of an oligonucleotide sequence selected from any one of SEQ ID NO: 4. In some embodiments, the oligonucleotide described herein includes a sequence that is at least 70%, 75%, 80%, 85%, 90%, 95%, 96%, 97%, 98%, 99%, or 100% identical to the full length of an oligonucleotide sequence selected from any one of SEQ ID NO: 5. In some embodiments, the oligonucleotide described herein includes a sequence that is at least 70%, 75%, 80%, 85%, 90%, 95%, 96%, 97%, 98%, 99%, or 100% identical to the full length of an oligonucleotide sequence selected from any one of SEQ ID NO: 6.

In some embodiments, the primers and probes described herein are used in a multiplex quantitative real-time PCR for the detection of the corresponding target genes. In some embodiments, the probes and primers for the detection of one or more genes, e.g., the human β-actin gene are used in combination for the detection and quantification of cfDNA.

In some embodiments, the oligonucleotides of the primers and probes described herein are modified (e.g., detectably labeled). In some embodiments, the oligonucleotide of probe for the detection of the human β-actin gene and the internal standard oligonucleotide is modified (e.g., detectably labeled). In some embodiments, the two ends of the probe (the 5′ end and the 3′ end) are respectively detectably labeled with a reporter fluorophore and a quenching fluorophore. The 5′ modifications of the probes of the invention are selected from the reporter fluorophores commonly used in the art, such as FAM, Texas Red, JOE; and the 3′ modification of the probe of the invention is selected from quenching fluorescent groups commonly used in the field, such as BHQ1, BHQ2, ECLIPSE. And the reporter fluorophore and the quencher fluorophore on the probe for a gene are different from the probe for another gene. Any other suitable oligonucleotide modifications can be used in the probes described herein. Any other suitable fluorophore and quencher fluorophore can be used to modify the probes described herein.

In some embodiments, the probe for the detection of the human β-actin gene is detectably labeled with JOE at the 5′ terminus and/or the probe for the detection of the human β-actin gene is detectably labeled with BHQ1 at the 3′ terminus. In some embodiments, the probe for the detection of the internal standard oligonucleotide is detectably labeled with FAM at the 5′ terminus and/or the probe for the detection of the internal standard oligonucleotide is detectably labeled with BHQ1 at the 3′ terminus.

Also provided herein are pharmaceutical compositions including an effective amount of the oligonucleotide described herein, and a pharmaceutically acceptable carrier, diluent, or both.

### Methods of Detection

Provided herein are methods including contacting a biological sample with the oligonucleotide described herein. In some embodiments, the methods further include detecting and quantifying a cfDNA (e.g., by detecting and quantifying a human β-actin gene) in the biological sample.

Accordingly, provided herein are methods for detecting cell-free DNA in a biological sample, wherein said method includes: (A) incubating the biological sample with: (1) a DNA polymerase and dNTP; and (2) a forward primer for target human gene; (3) a reverse primer for a target human gene; and (4) a detectably labeled probe, wherein the probe comprises an oligonucleotide sequence that is able to specifically hybridize to an oligonucleotide sequence of the target human gene; wherein the incubation is in a reaction under conditions sufficient to permit the forward and reverse primers to mediate a polymerase chain reaction amplification of a region of the target human gene to thereby produce an amplified target human gene fragment, if said target human gene is present in said clinical sample; and (B) detecting the target human gene; thereby detecting the presence of cell-free DNA in the biological sample.

Any suitable primer(s), probe(s), and target human genes described herein can be used in the methods described herein.

Also provided herein are methods for detecting cell-free DNA in a biological sample, wherein said method includes: (A) incubating the biological sample with: (1) a DNA polymerase and dNTP; and (2) a forward primer for a human β-actin gene having a nucleotide sequence consisting of SEQ ID NO: 2; (3) a reverse primer for a human β-actin gene having a nucleotide sequence consisting of SEQ ID NO: 3; and (4) a detectably labeled probe, wherein the probe comprises an oligonucleotide sequence that is able to specifically hybridize to an oligonucleotide sequence of human β-actin gene; wherein the incubation is in a reaction under conditions sufficient to permit the forward and reverse primers to mediate a polymerase chain reaction amplification of a region of the human β-actin gene to thereby produce an amplified human β-actin fragment, if said human β-actin gene is present in said clinical sample; and (B) detecting the human β-actin gene; thereby detecting the presence of cell-free DNA in the biological sample.

In some embodiments, the methods described herein further include quantifying the human β-actin gene in the biological sample if said human β-actin gene is present in said clinical sample.

Any suitable probes described herein can be used for detecting the human β-actin gene in the biological sample. In some embodiments, the probe for detecting the human β-actin gene includes an oligonucleotide sequence of SEQ ID NO: 5. In some embodiments, the probe for the detection of the human β-actin gene is detectably labeled with JOE at the 5′ terminus and/or the probe for the detection of the human β-actin gene is detectably labeled with BHQ1 at the 3′ terminus.

A biological sample as used herein includes any relevant biological sample that can be used for the detection and quantification of cfDNA. In some embodiments, the biological sample is obtained from a human subject. Bodily fluid includes blood and blood fractions or products (e.g., serum, buffy coat, plasma, platelets, red blood cells, and the like), mucosal secretions, such as with no limitations oral and respiratory tract secretions (sputa, saliva and the like), urine, malignant effusion, and other bodily fluids (e.g., prostatic fluid, gastric fluid, intestinal fluid, renal fluid, lung fluid, cerebrospinal fluid, and the like), etc.

In some particular embodiments, the biological sample is a clinical sample from a human individual having or suspected of having a disease or disease-related condition (e.g., for SARS-COV-2 patients, preferably a bodily fluid sample, more preferably oral or respiratory tract secretions).

The disease or disease-related condition described herein can be any disease that causes a change of level of cell-free DNA in the subject. For example, the disease or the condition can be a blood disease (e.g., haematological diseases, anaemia, clotting (including thromboses and venous embolisms) and abnormal development and function of platelets and erythrocytes); a cancer and neoplasms such as benign, potentially malignant, or malignant (cancer) cancer growths, leukemia and mesothelioma; a cardiovascular disease such as a coronary heart disease, diseases of the vasculature and circulation including the lymphatic system, and abnormal development and function of the cardiovascular system; an ear condition such as injury; an eye condition such as injury; an infection such as diseases caused by pathogens, acquired immune deficiency syndrome, and sexually transmitted infections; an inflammatory and immune system disease such as rheumatoid arthritis, connective tissue diseases, autoimmune diseases, allergies and abnormal development and function of the immune system; injuries and accidents such as trauma, fractures, poisoning and burns; a metabolic and endocrine disease such as metabolic disorders (including diabetes) and abnormality on the pineal, thyroid, parathyroid, pituitary and adrenal glands; a musculoskeletal disease such as osteoporosis, osteoarthritis, muscular and skeletal disorders; a neurological disease such as dementias, transmissible spongiform encephalopathies, Parkinson's disease, neurodegenerative diseases, Alzheimer's disease, epilepsy, and multiple sclerosis; an oral and gastrointestinal disease such as inflammatory bowel disease, Crohn's disease, diseases of the mouth, teeth, esophagus, and digestive system including liver and colon; a renal and urogenital disease such as kidney disease, pelvic inflammatory disease, and renal and genital disorders; a reproductive health and childbirth-related disease such as abortion; a respiratory disease such as asthma, chronic obstructive pulmonary disease, and respiratory diseases; a skin disease such as dermatological conditions; and a stroke including both ischemic stroke (caused by blood clots) and hemorrhagic stroke (caused by cerebral/intracranial hemorrhage).

The disease or condition can be one or more of cancer, normal pregnancy, a complication of pregnancy (e.g., aneuploid pregnancy), myocardial infarction, inflammatory bowel disease, systemic autoimmune disease, localized autoimmune disease, allotransplantation with rejection, allotransplantation without rejection, stroke, and localized tissue damage

In some embodiments, the disease or the disease-related condition can be one or more of a cancer or tumor, an infection (a bacterial or viral infection), a transplantation (e.g., an organ transplantation that results in graft rejection), tissue damage, or inflammation such as systemic lupus, kidney injury, and haemodialysis. The details of such diseases or the disease-related conditions can be found, for example, in Celec, P. et al., (2018). Cell-free DNA: The role in pathophysiology and as a biomarker in kidney diseases. Expert Reviews in Molecular Medicine, 20, E1. doi: 10.1017/erm.2017.12. In some embodiments, the disease is a hepatitis. In some embodiments, the disease is a hepatitis B-related liver disease. In some embodiments, the disease is a sepsis. In some embodiments, the biological sample is a clinical sample from a human individual undergoing a chemotherapy for cancer treatment. In some embodiments, the cancer or tumor comprises an acute lymphoblastic leukemia; acute myeloid leukemia; adrenocortical carcinoma; AIDS-related cancer; AIDS-related lymphoma; anal cancer; appendix cancer; astrocytomas; atypical teratoid/rhabdoid tumor; basal cell carcinoma; bladder cancer; brain stem glioma; brain tumor, brain stem glioma, central nervous system atypical teratoid/rhabdoid tumor, central nervous system embryonal tumors, astrocytomas, craniopharyngioma, ependymoblastoma, ependymoma, medulloblastoma, medulloepithelioma, pineal parenchymal tumors of intermediate differentiation, supratentorial primitive neuroectodermal tumors and pineoblastoma; breast cancer; bronchial tumors; Burkitt lymphoma; cancer of unknown primary site (CUP); carcinoid tumor; carcinoma of unknown primary site; central nervous system atypical teratoid/rhabdoid tumor; central nervous system embryonal tumors; cervical cancer; childhood cancers; chordoma; chronic lymphocytic leukemia; chronic myelogenous leukemia; chronic myeloproliferative disorders; colon cancer; colorectal cancer; craniopharyngioma; cutaneous T-cell lymphoma; endocrine pancreas islet cell tumors; endometrial cancer; ependymoblastoma; ependymoma; esophageal cancer; esthesioneuroblastoma; Ewing sarcoma; extracranial germ cell tumor; extragonadal germ cell tumor; extrahepatic bile duct cancer; gallbladder cancer; gastric (stomach) cancer; gastrointestinal carcinoid tumor; gastrointestinal stromal cell tumor; gastrointestinal stromal tumor (GIST); gestational trophoblastic tumor; glioma; hairy cell leukemia; head and neck cancer; heart cancer; Hodgkin lymphoma; hypopharyngeal cancer; intraocular melanoma; islet cell tumors; Kaposi sarcoma; kidney cancer; Langerhans cell histiocytosis; laryngeal cancer; lip cancer; liver cancer; malignant fibrous histiocytoma bone cancer; medulloblastoma; medulloepithelioma; melanoma; Merkel cell carcinoma; Merkel cell skin carcinoma; mesothelioma; metastatic squamous neck cancer with occult primary; mouth cancer; multiple endocrine neoplasia syndromes; multiple myeloma; multiple myeloma/plasma cell neoplasm; mycosis fungoides; myelodysplastic syndromes; myeloproliferative neoplasms; nasal cavity cancer; nasopharyngeal cancer; neuroblastoma; Non-Hodgkin lymphoma; nonmelanoma skin cancer; non-small cell lung cancer; oral cancer; oral cavity cancer; oropharyngeal cancer; osteosarcoma; other brain and spinal cord tumors; ovarian cancer; ovarian epithelial cancer; ovarian germ cell tumor; ovarian low malignant potential tumor; pancreatic cancer; papillomatosis; paranasal sinus cancer; parathyroid cancer; pelvic cancer; penile cancer; pharyngeal cancer; pineal parenchymal tumors of intermediate differentiation; pineoblastoma; pituitary tumor; plasma cell neoplasm/multiple myeloma; pleuropulmonary blastoma; primary central nervous system (CNS) lymphoma; primary hepatocellular liver cancer; prostate cancer; rectal cancer; renal cancer; renal cell (kidney) cancer; renal cell cancer; respiratory tract cancer; retinoblastoma; rhabdomyosarcoma; salivary gland cancer; Sézary syndrome; small cell lung cancer; small intestine cancer; soft tissue sarcoma; squamous cell carcinoma; squamous neck cancer; stomach (gastric) cancer; supratentorial primitive neuroectodermal tumors; T-cell lymphoma; testicular cancer; throat cancer; thymic carcinoma; thymoma; thyroid cancer; transitional cell cancer; transitional cell cancer of the renal pelvis and ureter; trophoblastic tumor; ureter cancer; urethral cancer; uterine cancer; uterine sarcoma; vaginal cancer; vulvar cancer; Waldenström macroglobulinemia; or Wilm's tumor.

In some embodiments, the biological sample is a clinical bodily fluid sample from a human individual suffered from a physical trauma, which may be caused by accidents, falls, hits, weapons, and other causes. For example, a physical trauma can be a wound, an injury in which skin is torn, cut or punctured (an open wound), or where blunt force trauma causes a contusion (a closed wound), a head injury, a penetrating head injury, a closed head injury, an eye injury, a chemical eye injury, an eye injuries during general anaesthesia, a brain injury, an acquired brain injury, a coup countercoup injury, a diffuse axonal injury, a frontal lobe injury, a nerve injury, a spinal cord injury, a brachial plexus injury, a sciatic nerve injury, an injury of axillary nerve, a soft tissue injury, a tracheobronchial injury, an acute kidney injury, an anterior cruciate ligament injury, a musculoskeletal injury, articular cartilage injuries, an acute lung injury, a pancreatic injury, a thoracic aorta injury, a biliary injury, a lisfranc injury, a knee injury, medial knee injuries, a back injury, a hand injury, and/or a chest injury. In some embodiments, the methods described herein further includes assessing the severity of the disease or injury of the subject based on the detection and quantification of the cfDNA.

The biological sample can be subjected to well-known isolation and purification protocols or used directly. For example, the sample can be subjected to a treatment to release/extract the nucleic acids of the sample and/or to remove proteins and other non-nucleic acid components of the sample using conventional techniques.

In some embodiments, the biological sample used for the detection and quantification of cfDNA is essentially free of cellular DNA (i.e., DNA contained within cells or cellular compartments). For example, the biological sample used in the methods described herein can be subject to one or more steps that remove cellular DNA (i.e., DNA within intact cells or cellular compartments).

Any suitable methods can be used to remove cellular DNA from the biological sample. In some embodiments, the cellular DNA is removed using centrifugation, microfluidic-based separation, columns or magnetic beads, phenol-chloroform-based separation, or filtration-based separation (see, e.g., Wan, J. C. et al. Liquid biopsies come of age: towards implementation of circulating tumour DNA. Nat. Rev. Cancer 17, 223 (2017); and Hoyoon Lee et al., npj Precision Oncology volume 4, Article number: 3 (2020)).

In some embodiments, the removing of cellular DNA is performed before the detection and/or quantification of cfDNA.

The biological sample that is essentially free of cellular DNA contains no more than about 30%, about 25%, about 20%, about 15%, about 10%, about 5%, about 4%, about 3%, about 2%, about 1%, or less cellular DNA.

In some embodiments, the probes used in the methods (e.g., the probe for human β-actin gene, or the probe for the internal standard oligonucleotide) hybridizes to the amplified fragments of the corresponding targets.

In some embodiments, the DNA polymerase used in the methods has a 5′3′ exonuclease activity that hydrolyzes the hybridized probes (e.g., the probe for human β-actin gene, or the probe for the internal standard oligonucleotide) to thereby separate the detectable labels on the probes and cause a signal to become detected. In some embodiments, the DNA polymerase is a Taq DNA polymerase. In some embodiments, the DNA polymerase is a hot start Taq DNA polymerase.

In some embodiments, the signal is a fluorescent signal. In some embodiments, the hybridization of the probes to the amplified fragments of the target genes separates the detectable labels on the probe and causes a signal to become detectable. In some embodiments, the signal is a fluorescent signal. Other suitable methods of detectably label a probe and detecting the signals are known in the art.

In some embodiments, the ratio of the final concentration of each primer to the final concentration of the probe used in the reaction is about 5:1, 4:1, 3:1, 2:1, 1:1, 1:2, 1:3, 1:4, or about 1:5. In some embodiments, the ratio of the final concentration of each primer to the final concentration of the probe used in the reaction is about 2:1.

In some embodiments, an amount of internal standard oligonucleotide is added to the biological sample as a quantification standard. The detection and quantification of the internal standard oligonucleotide can be used to generate a standard curve for the quantification of the human β-actin gene, which in turn can be used for the quantification of the cfDNA in the biological sample. In some embodiments, a standard curve is not used for the detection and/or quantification of cfDNA.

Accordingly, in some embodiments, the method described herein further comprises: (C) adding an amount of internal standard oligonucleotides to the biological sample; (D) incubating the biological sample in (C) with: (1) a DNA polymerase and dNTP; and (2) a forward primer having a nucleotide sequence consisting of SEQ ID NO: 4; (3) a reverse primer having a nucleotide sequence consisting of SEQ ID NO: 3; and (4) a detectably labeled internal standard probe, wherein the probe comprises an oligonucleotide sequence that is able to specifically hybridize to the internal standard oligonucleotide; wherein the incubation is in a reaction under conditions sufficient to permit the forward and reverse primers to mediate a polymerase chain reaction amplification of a region of the internal standard oligonucleotide to thereby produce an amplified fragment of the region; and (E) detecting the internal standard oligonucleotides.

In some embodiments, the subject is a human subject.

Any internal standard oligonucleotide described herein can be used in the method. In some embodiments, the internal standard oligonucleotide has a sequence of SEQ ID NO: 1.

In some embodiments, the internal standard probe comprises an oligonucleotide sequence of SEQ ID NO: 6. In some embodiments, the internal standard probe is detectably labeled with FAM at the 5′ terminus and/or wherein the internal standard probe is detectably labeled with BHQ1 at the 3′ terminus. In some embodiments, the internal standard probe hybridizes to the amplified fragments of the internal standard oligonucleotide.

The amount of the internal standard added to the biological sample is important to the accuracy and amplification efficiency of the cfDNA (e.g., the human β-actin gene). In some embodiments, about 5×10², 1×10³, 5×10³, 1×10⁴, 2×10⁴, 3×10⁴, 4×10⁴, 5×10⁴, 6×10⁴, 7×10⁴, 8×10⁴, 9×10⁴, 1 ×10⁵, 5×10⁵, 1 ×10⁶ or more copies of the internal standard oligonucleotides are added in to each 195 μL biological sample. In some embodiments, about 5×10⁴ copies of the internal standard oligonucleotides are added in to each 195 mL biological sample.

In some embodiments, the internal standard oligonucleotides (e.g., about 5×10⁴ copies of the internal standard oligonucleotides) in a volume of about 1 μL, 2 μL, 3 μL, 4 μL, 5 μL, 6 μL, 7 μL, 8 μL, 9 μL, 10 UL or more to each 195 μL of the biological sample. In some embodiments, the internal standard oligonucleotides (e.g., about 5×10⁴ copies of the internal standard oligonucleotides) are added to the biological sample in a volume of about 5 μL to each 195 μL of the biological sample. In some embodiments, the internal standard oligonucleotides (about 5×10⁴ copies of the internal standard oligonucleotides) are added to the biological sample at a final concentration (v/v) of about 1:20, 1:25, 1:30, 1:35, 1:40, 1:45 or 1:50. In a preferred embodiment, the internal standard oligonucleotides are added to the biological sample at a final concentration (v/v) of about 1:40.

In some embodiments, the cfDNA (e.g., the human β-actin gene) is detected at a DNA concentration of about 1 ng/mL, about 5 ng/ml, about 10 ng/ml, about 50 ng/ml, about 100 ng/mL, about 200 ng/mL, about 300 ng/mL, about 400 ng/mL, about 500 ng/ml, about 600 ng/mL, about 700 ng/mL, about 800 ng/ml, about 900 ng/mL, about 1000 ng/ml, about 2000 ng/ml, about 3000 ng/mL, about 4000 ng/mL, about 5000 ng/mL, about 10000 ng/ml or higher. In some embodiments, the cfDNA (e.g., the human β-actin gene) is detected at a concentration of at least 1 ng/ml (e.g., with a sensitivity of at least 95%). Because of the optimization of the probes, the limit of detection of the methods described herein is about 1 ng/mL.

In some embodiments, the internal standard oligonucleotide is detected at a DNA concentration of about 1 ng/mL, about 5 ng/mL, about 10 ng/ml, about 50 ng/ml, about 100 ng/mL, about 200 ng/mL, about 300 ng/mL, about 400 ng/mL, about 500 ng/mL, about 600 ng/mL, about 700 ng/mL, about 800 ng/ml, about 900 ng/ml, about 1000 ng/ml, about 2000 ng/mL, about 3000 ng/mL, about 4000 ng/ml, about 5000 ng/mL, about 10000 ng/ml or higher. In some embodiments, the internal standard oligonucleotide is detected at a concentration of at least 1 ng/ml (e.g., with a sensitivity of at least 95%). Because of the optimization of the probes, the limit of detection of the methods described herein is about 1 ng/mL.

Other suitable positive or negative reference markers can also be used in the method described herein. For example, virus preservation solutions can be used as a negative reference for the detection of SARS-COV-2 infection.

Also provided herein is a method for quantifying cell-free DNA in a biological sample, wherein said method comprises:
(A) incubating the biological sample with:
(1) a DNA polymerase and dNTP;
(2) a forward primer for a human β-actin gene having a nucleotide sequence consisting of SEQ ID NO: 2;
(3) a reverse primer for a human β-actin gene having a nucleotide sequence consisting of SEQ ID NO: 3;
(4) a detectably labeled probe, wherein the probe comprises an oligonucleotide sequence that is able to specifically hybridize to an oligonucleotide sequence of human β-actin gene;
wherein the incubation is in a reaction under conditions sufficient to permit the forward and reverse primers to mediate a polymerase chain reaction amplification of a region of the human β-actin gene to thereby produce an amplified human β-actin fragment, if said human β-actin gene is present in said clinical sample;
(B) adding an amount of internal standard oligonucleotides having a sequence of SEQ ID NO: 1 to the biological sample;
(C) incubating the biological sample in (B) with:
(1) a DNA polymerase and dNTP;
(2) a forward primer having a nucleotide sequence consisting of SEQ ID NO: 4;
(3) a reverse primer having a nucleotide sequence consisting of SEQ ID NO: 3;
(4) a detectably labeled probe, wherein the probe comprises an oligonucleotide sequence that is able to specifically hybridize to the internal standard oligonucleotide;
wherein the incubation is in a reaction under conditions sufficient to permit the forward and reverse primers to mediate a polymerase chain reaction amplification of a region of the sequence of SEQ ID NO: 1 to thereby produce an amplified fragment of the region;
(D) detecting the internal standard oligonucleotide;
(E) detecting and quantifying the human β-actin gene based on the detection of the internal standard oligonucleotide;
thereby quantifying the cell-free DNA in the biological sample.

### Prediction of Disease Severity and Progression

The disclosure features methods for determining the severity of one or more diseases, e.g., a SARS-COV-2 infection (used interchangeably with coronavirus disease 2019, or COVID-19), in a subject. The methods include obtaining a sample from the subject; determining the level of cell-free DNA (cfDNA) in the sample; and comparing the cfDNA level in the sample to a cut-off value. The cfDNA level in the sample as compared to the cut-off value indicates whether the one or more diseases the subject has are severe, e.g., life-threatening.

In a further aspect, the disclosure includes methods for monitoring a subject's condition, e.g., for deciding whether a subject has improved, e.g., improved sufficiently to be discharged from the hospital. The methods include determining a first cfDNA level in the subject, e.g., a baseline level; and determining at least one subsequent cfDNA level in the subject, e.g., a treatment level. Then, the first level and the subsequent levels are compared. If the biomarker level of cfDNA decreases sufficiently, e.g., statistically significantly, or by at least 5%, 10%, 15%, 20%, or more, from the first to the subsequent levels, then the subject's condition is likely to be improving and, if either one or both levels are low enough, e.g., below a selected threshold, then the subject can be discharged, e.g., for outpatient treatment.

The methods described herein can be used to predict the progression of a disease or disorder. In some embodiments, the methods described herein can be used to predict the severity of a disease or disorder. Methods of determining and monitoring disease progression can be found, for example, at PCT Publication No. WO 2007/127749A3, the entire content of which is incorporated herein. In some embodiments, the disease or disorder is a SARS-COV-2 infection.

Autologous circulating cell-free DNA (cfDNA) as a biomarker of cell death typically remains a low level in plasma of healthy people. While in the situations such as sepsis, trauma, malignancies and endotheliopathy, abnormal cell death occurs, leading to tissue damage even organ failure, and the intracellular genomic DNA is released and absorbed into circulation, significantly increasing the level of cfDNA in plasma. The novel duplex real-time PCR assay using an internal standard oligonucleotide described herein eliminates the pre-analytical errors while increasing the precision and accuracy for autologous cfDNA quantification in plasma. Thus, quantifying plasma DNA using the novel assay described herein has certain advantages for the assessment of disease severity and progression in patients infected by SARS-COV-2 (COVID-19 patients).

Accordingly, in one aspect, provided herein is a method of predicting the severity of an infection by SARS-COV-2, comprising: (A) obtaining a biological sample from a subject having a SARS-COV-2 infection; (B) quantifying cell-free DNA (cfDNA) in the biological sample; and (C) predicting the severity based on the quantification of the cfDNA, wherein a cfDNA concentration above a cut-off value indicates deterioration of the SARS-COV-2 infection.

Any methods described herein for the detection and quantification of cfDNA can be used in the methods for predicting the severity of the SARS-COV-2 infection. In some embodiments, the quantification of the cfDNA includes quantifying a housekeeping gene in the biological sample. In some embodiments, the housekeeping gene is a human β-actin gene.

In some embodiments, the quantification of the cfDNA includes incubating the biological sample with: (1) a DNA polymerase and dNTP; (2) a forward primer for a human β-actin gene; (3) a reverse primer for a human β-actin gene; (4) a detectably labeled probe, wherein the probe comprises an oligonucleotide sequence that is able to specifically hybridize to an oligonucleotide sequence of human β-actin gene; wherein the incubation is in a reaction under conditions sufficient to permit the forward and reverse primers to mediate a polymerase chain reaction amplification of a region of the human β-actin gene to thereby produce an amplified human β-actin fragment, if said human β-actin gene is present in said clinical sample.

Any suitable primers and probes described herein can be used in the quantification of the human β-actin gene. In some embodiments, the forward primer for the human β-actin gene has a nucleotide sequence that is at least 80%, 85%, 90%, 95, 99% or 100% identical to SEQ ID NO: 2. In some embodiments, the reverse primer for the human β-actin gene has a nucleotide sequence that is at least 80%, 85%, 90%, 95, 99% or 100% identical to SEQ ID NO: 3.

In some embodiments, the detectably labeled probe for human β-actin gene has a sequence that is at least 80%, 85%, 90%, 95, 99% or 100% identical to SEQ ID NO: 5.

In some embodiments, the quantification of the cfDNA includes adding an amount of an internal standard oligonucleotides to the biological sample; and incubating the biological sample with: (1) a DNA polymerase and dNTP; (2) a forward primer for the internal standard oligonucleotide; (3) a reverse primer for the internal standard oligonucleotide; (4) a detectably labeled probe, wherein the probe comprises an oligonucleotide sequence that is able to specifically hybridize to the internal standard oligonucleotide; wherein the incubation is in a reaction under conditions sufficient to permit the forward and reverse primers to mediate a polymerase chain reaction amplification of a region of the internal standard oligonucleotide to thereby produce an amplified fragment of the amplified region.

Any suitable primers and probes described herein can be used in the detection of the internal standard oligonucleotide. In some embodiments, the forward primer for the internal standard oligonucleotide has a nucleotide sequence that is at least 80%, 85%, 90%, 95, 99% or 100% identical to SEQ ID NO: 4. In some embodiments, the reverse primer for the internal standard oligonucleotide has a nucleotide sequence that is at least 80%, 85%, 90%, 95, 99% or 100% identical to SEQ ID NO: 3.

In some embodiments, the detectably labeled probe for the internal standard oligonucleotide has a sequence that is at least 80%, 85%, 90%, 95, 99% or 100% identical to SEQ ID NO: 6.

In some embodiments, the internal standard oligonucleotide has a sequence that is at least 80%, 85%, 90%, 95, 99% or 100% identical to SEQ ID NO: 1.

In some embodiments, the methods described herein further includes detecting and quantifying the human β-actin gene based on the detection of the internal standard oligonucleotide.

The cut-off value as described herein is a level, or a range of levels of the cfDNA used to determine the severity of the disease, e.g., SARS-COV-2 infection. In some embodiments, if the cfDNA level in the biological sample is above the cut-off value, it is predicted that the disease (e.g., SARS-COV-2 infection) will deteriorate. In some embodiments, if the cfDNA level in the biological sample is above the cut-off value, it is determined that the disease (e.g., SARS-COV-2 infection) is a severe status.

In some embodiments, if the cfDNA level in the biological sample is below the cut-off value, it is predicted that the disease (e.g., SARS-COV-2 infection) will not deteriorate. In some embodiments, if the cfDNA level in the biological sample is below the cut-off value, it is determined that the disease (e.g., SARS-COV-2 infection) is a non-severe status.

The cut-off values used in the methods described herein are novel and specific for the prediction of the disease or disorder (e.g., SARS-COV-2 infection) progression. In some embodiments, the cut-off value used in the methods described herein is about 10 ng/ml to about 1000 ng/ml, about 10 ng/ml to about 900 ng/ml, about 10 ng/ml to about 800 ng/ml, about 10 ng/ml to about 700 ng/ml, about 10 ng/ml to about 600 ng/ml, about 10 ng/ml to about 500 ng/ml, about 10 ng/ml to about 400 ng/ml, about 10 ng/ml to about 300 ng/ml, about 10 ng/ml to about 200 ng/ml, about 90 ng/ml to about 1000 ng/ml, about 90 ng/ml to about 900 ng/ml, about 90 ng/ml to about 800 ng/ml, about 90 ng/ml to about 700 ng/ml, about 90 ng/ml to about 600 ng/ml, about 90 ng/ml to about 500 ng/ml, about 90 ng/ml to about 400 ng/ml, or about 90 ng/ml to about 300 ng/ml.

In some embodiments, the cut-off value is 90 ng/ml to about 350 ng/ml. In some embodiments, the cut-off value is about 90 ng/ml to about 300 ng/ml. In some embodiments, the cut-off value is about 90 ng/ml to about 250 ng/ml. In some embodiments, the cut-off value is about 90 ng/ml to about 200 ng/ml. In some embodiments, the cut-off value is about 150 ng/ml to about 200 ng/ml. In some embodiments, the cut-off value used in the methods described herein is about or at least 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, or 350 ng/ml. In some embodiments, the cut-off value used in the methods described herein is no more than 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, or 350 ng/ml. In some embodiments, the cut-off value is about 165 to about 175 ng/ml. In some embodiments, the cut-off value is 169.3 ng/ml.

The prediction of the severity of the disease (e.g., SARS-COV-2) described herein can be based on further predictor(s) used in clinical diagnosis and treatment. For example, the prediction of the severity of the SARS-COV-2 infection can be based on one or more of demographic variables, clinical signs and symptoms, imaging results, laboratory findings, and medical history.

In some embodiments, the severity of the SARS-COV-2 infection is determined as severe. In some embodiments, the severity of the SARS-COV-2 infection is determined as non-severe. In some embodiments, the severe status of the SARS-COV-2 infection corresponds to the deterioration of the subject's condition. In some embodiments, the methods described herein predicts the deterioration of the subject's condition. In some embodiments, deterioration is one or more of abnormally high or low body temperature; usage or increment of vasoactive, sedative, and analgesic agents; respiration supports upgrading; increasing of visible or occult bleeding; and development of additional complications. In some embodiments, the deterioration of the subject's condition is indicated by a cfDNA level above the cut-off value described herein. In some embodiments, the deterioration of the subject's condition corresponds to one or more symptoms of a severe condition of a SARS-COV-2 infection. In some embodiments, the methods described herein predict the deterioration of the condition of a subject who has other clinical manifestation of a severe condition. In some embodiments, the methods described herein predict the deterioration of the condition of a subject who does not have other clinical manifestation of a severe condition.

Any suitable clinical signs and symptoms can be used herein. In some embodiments, the clinical signs and symptoms are selected from body temperature, systolic blood pressure, diastolic blood pressure, heart rate, respiratory rate, vasoactive agents administration, sedative agents administration, analgesic agents administration and unconsciousness.

Methods of obtaining and interpreting imaging results are known in the art. In some embodiments, the imaging result is abnormality of chest radiography and/or computed tomography (CT) imaging.

Laboratory testing can be used in combination with the methods described herein to predict the severity of the SARS-COV-2 infection. Suitable laboratory testing is known in the art. In some embodiments, the laboratory findings are selected from partial arterial oxygen pressure, oxygen saturation, white blood cell counts and differentiation, neutrophil to lymphocyte ratio (NLR), platelet counts, hematocrit, serum sodium and potassium, pH, total bilirubin, creatinine, and D-dimer levels (degradation products of cross-linked fibrin, whose level becomes elevated following clot formation).

The medical history of the subject is also important in determining and predicting the severity of the SARS-COV-2 infection. In some embodiments, the medical history is selected from past operation, chronic obstructive pulmonary disease, liver cirrhosis, renal dialysis, immunodeficiency disease, cancer, chemotherapy, radiation, long term and high dose steroids.

Other standards used in SARS-COV-2 diagnosing and severity determination can also be used in combination with the methods described herein. For example, the determination of the level of severity can be based on the COVID-19 Treatment Guidelines published by the National Institutes of Health (NIH) (see, e.g., NIH website: covid19treatmentguidelines.nih.gov/management/clinical-management/hospitalized-adults-therapeutic-management/hospitalized-adults-figure/; and World Health Organization, Clinical management of COVID-19: interim guidance, May 27, 2020).

Specifically, according to the NIH guideline, patients with SARS-COV-2 infection can experience a range of clinical manifestations, from no symptoms to critical illness. In general, adults with SARS-COV-2 infection can be grouped into the following severity of illness categories; however, the criteria for each category may overlap or vary across clinical guidelines and clinical trials, and a patient's clinical status may change over time (see, e.g., NIH website: covid19treatmentguidelines.nih.gov/overview/clinical-spectrum/).
Asymptomatic or Presymptomatic Infection: Individuals who test positive for SARS-CoV-2 using a virologic test (i.e., a nucleic acid amplification test [NAAT] or an antigen test) but who have no symptoms that are consistent with COVID-19.
Mild Illness: Individuals who have any of the various signs and symptoms of COVID-19 (e.g., fever, cough, sore throat, malaise, headache, muscle pain, nausea, vomiting, diarrhea, loss of taste and smell) but who do not have shortness of breath, dyspnea, or abnormal chest imaging.
Moderate Illness: Individuals who show evidence of lower respiratory disease during clinical assessment or imaging and who have an oxygen saturation (SpO₂)≥94% on room air at sea level.
Severe Illness: Individuals who have SpO₂<94% on room air at sea level, a ratio of arterial partial pressure of oxygen to fraction of inspired oxygen (PaO₂/FiO₂)<300 mm Hg, a respiratory rate >30 breaths/min, or lung infiltrates >50%.
Critical Illness: Individuals who have respiratory failure, septic shock, and/or multiple organ dysfunction.

Patients with certain underlying comorbidities are at a higher risk of progressing to severe COVID-19. These comorbidities include being aged >65 years; having cardiovascular disease, chronic lung disease, sickle cell disease, diabetes, cancer, obesity, or chronic kidney disease; being pregnant; being a cigarette smoker; being a transplant recipient; and receiving immunosuppressive therapy. Health care providers should monitor such patients closely until clinical recovery is achieved.

According to the WHO guideline (World Health Organization, Clinical management of COVID-19: interim guidance, May 27, 2020), the severity of SARS-COV-2 is determined using the following categories: mild disease, moderate disease (pneumonia), severe disease (severe pneumonia), critical disease (acute respiratory distress syndrome (ARDS)), and critical disease (sepsis or septic shock).

In some embodiments, the sever status described herein corresponds to severe illness or critical illness described in the NIH guideline. In some embodiments, the sever status described herein corresponds to severe disease (severe pneumonia), critical disease (acute respiratory distress syndrome (ARDS)), or critical disease (sepsis or septic shock) described in the WHO guideline.

In some embodiments, the non-sever status described herein corresponds to asymptomatic or presymptomatic infection, mild illness or moderate illness described in the NIH guideline. In some embodiments, the sever status described herein corresponds to mild disease or moderate disease (pneumonia) described in the WHO guideline.

The optimal pulmonary imaging technique has not yet been defined for people with symptomatic SARS-COV-2 infection. Initial evaluation for these patients may include a chest X-ray, ultrasound screening, or, if indicated, a computed tomography scan. An electrocardiogram should be performed if indicated. Laboratory testing includes a complete blood count with differential and a metabolic profile, including liver and renal function tests.

Although inflammatory markers such as C-reactive protein (CRP), D-dimer, and ferritin are not routinely measured as part of standard care, results from such measurements may have prognostic value.

In some embodiments, the prediction of the severity of the SARS-COV-2 infection further comprises calculating the Acute Physiology and Chronic Health Evaluation

(APACHE II) and/or Sequential Organ Failure Assessment (SOFA) scores on the worst value for one or more physiological variables.

In some embodiments, the calculation of the APACHE II and SOFA scores is performed within 24 hours of the time point when the biological sample is collected.

The Acute Physiology and Chronic Health Evaluation (APACHE II) is a severity score and mortality estimation tool developed from a large sample of ICU patients in the United States (see, e.g., Knaus W A et al., APACHE II: a severity of disease classification system. Crit Care Med. 1985; 13 (10): 818-29). The APACHE II score is made of 12 physiological variables and 2 disease-related variables. Within the study period, 87% of all ICU patients had all 12 physiologic measurements available. The worst physiological variables were collected within the first 24 hours of ICU admission. The “worst” measurement was defined as the measure that correlated to the highest number of points. The APACHE II score ranges from 0 to 71 points, with higher points correlating with higher predicted mortality.

In some embodiments, the deterioration of the SARS-COV-2 infection corresponds to an APACHE II score of 0 to 30. In some embodiments, the deterioration of the SARS-COV-2 infection corresponds to an APACHE II score of 10 to 20.

In some embodiments, the non-severe status of the SARS-COV-2 infection corresponds to an APACHE II score of 0 to 15. In some embodiments, the non-severe status of the SARS-COV-2 infection corresponds to an APACHE II score of 4 to 10.5.

The Sequential Organ Failure Assessment (SOFA) score is a scoring system that assesses the performance of several organ systems in the body (neurologic, blood, liver, kidney, and blood pressure/hemodynamics) and assigns a score based on the data obtained in each category. The higher the SOFA score, the higher the likely mortality (see, e.g., the online document files.asprtracie.hhs.gov/documents/aspr-tracie-sofa-score-fact-sheet.pdf).

In some embodiments, the deterioration of the SARS-COV-2 infection corresponds to SOFA score of 0 to 15. In some embodiments, the deterioration of the SARS-COV-2 infection corresponds to SOFA score of 4 to 9.

In some embodiments, the non-severe status of the SARS-COV-2 infection corresponds to a SOFA score of 0 to 5. In some embodiments, the non-severe status of the SARS-COV-2 infection corresponds to a SOFA score of 0 to 2.

In some embodiments, the sensitivity of the methods described herein is the rate of the methods described herein to predict the severe status in a subject.

In some embodiments, the prediction of the deterioration of the SARS-COV-2 infection has a sensitivity of at least 80%, 85%, 90%, 95%, 99% or higher. In some embodiments, the prediction of the deterioration of the SARS-COV-2 infection has a sensitivity of at least 85.0%

In some embodiments, the prediction of the deterioration of the SARS-COV-2 infection has a specificity of at least 80%, 85%, 90%, 95%, 99% or higher.

In some embodiments, the prediction of the deterioration of the SARS-COV-2 infection has a specificity of at least 86%.

The prediction of the disease severity described herein is especially useful for determining the treatment plan for patients with SARS-COV-2 infection. In some embodiments, the methods described herein further comprise determining a treatment plan for the SARS-COV-2 infection.

In some embodiments, the treatment plan for the deterioration of the SARS-COV-2 infection is selected from ICU admission, intratracheal intubation, hormone therapy, and extracorporeal membrane oxygenation (ECMO) treatment.

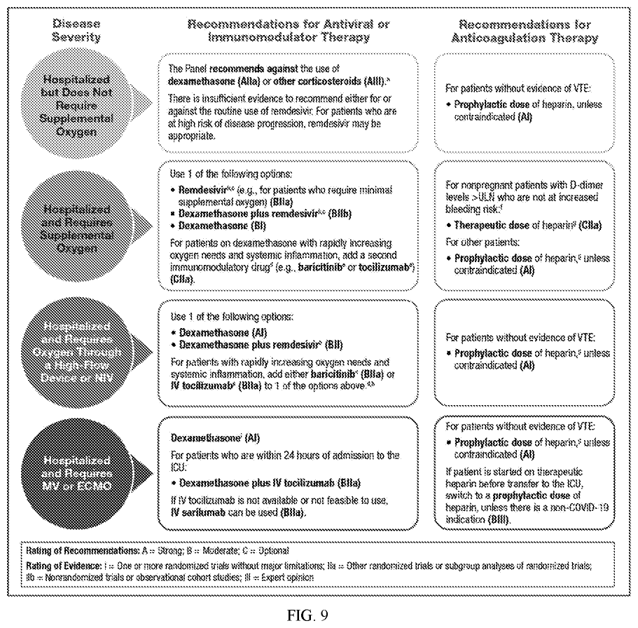

Suitable treatment for different levels of severity of SARS-COV-2 infection are known in the art. For example, the NIH Guideline for COVID-19 Treatment (FIG. 9, and NIH website: covid19treatmentguidelines.nih.gov/management/clinical-management/hospitalized-adults-therapeutic-management/hospitalized-adults-figure/) provides the treatment plans for disease severity including hospitalized but does not require supplemental oxygen, hospitalized and requires supplemental oxygen, hospitalized and requires oxygen through a high-flow device or NIV, and hospitalized and requires MV or ECMO.

In some embodiments, the treatment plan for the non-severe status of the SARS-COV-2 infection is selected from reducing the dosage of current administration of therapeutic agents, release from ICU, discharge from hospitalization.

The methods described herein can also be used to monitor the progression of the disease. In some embodiments, the methods described herein further include determining a baseline level of cfDNA in the biological sample. In some embodiments, the baseline level of cfDNA is used as a references level for comparison and determination of the severity of the disease.

In some embodiments, the methods described herein further include collecting one or more additional biological samples to determine the cfDNA level at one or more additional time points. For example, samples can be collected every 1, 2, 3, 4, 5, 6, 7, 9, 10, 11, 12, 13, 14, 15, 16, 17, 18, 19, 20, 21, 22, 23, or 24 hours; or every 1, 2, 3, 4, 5, 6, 7, 8, 9, 10 or more days for the monitoring the disease progression. In some embodiments, samples are collected every 4, 6, 8, 10, or 12 hours. In some embodiments, samples are collected every 4-6 hours.

In some embodiments, the methods described herein further include monitoring the levels of cfDNA from different time points over a certain time period. For example, the disease progression can be monitored over a period of about 1, 2, 3, 4, 5, 6, 7, 8, 9, 10 or more days.

The prediction of the severity of SARS-COV-2 disease can be the prediction of disease status for the next 1, 2, 3, 4, 5, 6, 7, 8, 9, 10 or more days. In some embodiments, the prediction of the severity of SARS-COV-2 disease is the prediction of disease status for the next 3-5 days. In some embodiments, the methods described herein predict that the subject's condition will deteriorate in the next 1, 2, 3, 4, 5, 6, 7, 8, 9, 10 or more days. In some embodiments, the methods described herein predict that the subject's condition will deteriorate in the next 3-5 days. In some embodiments, the methods described herein detect the deterioration of the subject's condition and predict one or more symptoms of a severe condition in the next 1, 2, 3, 4, 5, 6, 7, 8, 9, 10 or more days. In some embodiments, the methods described herein detect the deterioration of the subject's condition and predict one or more symptoms of a severe condition in the next 3-5 days. In some embodiments, the methods described herein detect the deterioration of the subject's condition and predict one or more symptoms of a severe condition in the next 3 days.

Any of the biological samples described herein can be used to predict and monitor the severity of the disease. In some embodiments, the biological sample is a plasma sample.

Quantification of cfDNA

Due to the optimization of the primer and probe sets for the detection and quantification of cfDNA, the methods described herein further improves the amplification efficiency of the cfDNA (e.g., the human β-actin gene) and the internal standard oligonucleotide. Thus, in some embodiments, the method described herein further includes determining the amplification efficiency of the internal standard oligonucleotide and the cfDNA (e.g., human β-actin gene).

Any suitable methods and algorithms used to determine the amplification efficiency of nucleic acid can be used in the methods described herein to determine the amplification efficiency of the cfDNA and internal standard oligonucleotide.

In some embodiments, the quantification of the cfDNA (e.g., human β-actin gene) is performed based on one or more of the parameters:
(1) The starting copy number of the internal standard oligonucleotide (S₀);
(2) The amplification efficiency of the cfDNA (e.g., human β-actin gene (E_{T}));
(3) The amplification efficiency of the internal standard oligonucleotide (E_{S});
(4) The cycle threshold for the cfDNA (e.g., human β-actin gene (Ct,T));
(5) The cycle threshold for the internal standard oligonucleotide (Ct,S) In some embodiments, the quantification of the cfDNA is represented by copy number per volume (e.g., copies/mL). In some embodiments, the concentration of cfDNA (T₀) is determined by adjusting the concentration of the internal standard oligonucleotide with the amplification efficiencies of the internal standard oligonucleotide and the target gene.

In some embodiments, the concentration of the cfDNA (e.g., human β-actin gene) can be determined by Formula (I):

$${T}_{0} = {S}_{0} \times \frac{{( 1 + {E}_{S} )}^{Ct , S}}{{( 1 + {E}_{T} )}^{Ct , T}} \qquad ( I )$$

wherein:
T₀ is the concentration by copy number (copies/mL) of the cfDNA. In some embodiments, the cfDNA is a human housekeeping gene. In some embodiments, the cfDNA is human β-actin gene;
S₀ is the concentration by copy number (copies/mL) of the internal standard oligonucleotide;
E_{T} is the PCR amplification efficiency of the cfDNA (e.g., human β-actin gene);
E_{S} is the PCR amplification efficiency of the internal standard oligonucleotide;
Ct,T is the Ct (cycle threshold) value for the cfDNA (e.g., human β-actin gene);
Ct,S is the Ct (cycle threshold) value for the internal standard oligonucleotide.

Described herein is an example of using the above formula for determining the concentration of the cfDNA. In this example, the PCR amplification efficiency for the cfDNA (e.g., human β-actin gene) is 99.71% (e.g., determined by the LinRegPCR software or serial dilutions) and the PCR amplification efficiency of the internal standard oligonucleotide is 99.88% (e.g., determined by the LinRegPCR software or serial dilutions), and 195 μL plasma sample is mixed with 1×10⁴ copies/μL internal standard oligonucleotide in a volume of 5 μL. Thus, every 1 mL mixture has

$$\frac{1 mL}{195 µL} \times 5 \times {10}^{4}$$

copies of the internal standard oligonucleotide, which results in S₀ of

$$\frac{1 mL}{195 µL} \times 5 \times {10}^{4}$$

copies/mL of the plasma sample. Based on the studies and references that the human haploid genome is about 3.3×10⁻³ ng, and every human haploid genomes contains one copy of the cfDNA (e.g., human β-actin gene), the concentration of the single-copy cfDNA (ng/mL) can be determined by Formula (II):

$$cfDNA concentration ( ng / mL ) = 84 \; 6.15 \times {2}^{( Ct , S - Ct , T )} \qquad ( II )$$

Wherein Ct,S and Ct,T are the Ct values of the internal standard oligonucleotide and the cfDNA (e.g., human β-actin gene), respectively, under the same threshold.

One of the advantages of the methods provided herein is the high sensitivity of detecting cfDNA, e.g., the ability of detecting the cfDNA (e.g., human β-actin gene) and the internal standard oligonucleotide with high Ct level.

In a real-time PCR assay a positive reaction is detected by accumulation of a fluorescent signal. The Ct (cycle threshold) is defined as the number of cycles required for the fluorescent signal to cross the threshold (i.e., exceeds background level). Ct levels are inversely proportional to the amount of target nucleic acid in the sample (i.e., the lower the Ct level the greater the amount of target nucleic acid in the sample).

### Kits

Also provided herein are cfDNA detection kits including the internal standard oligonucleotide, and the primers and probes for multiplex quantitative real-time PCR described herein.

Accordingly, provided herein is a kit, comprising:

(1) one or more internal standard oligonucleotide described herein; (2) a primer set described herein; (3) a PCR buffer solution, a DNA polymerase, MgCl₂, and dNTP; and (4) optionally instructions for performing any of the methods described herein.

In some embodiments, provided herein is a kit comprising: (1) a first composition comprising one or more internal standard oligonucleotide, wherein the one or more internal standard oligonucleotide comprises a sequence that is at least 90% identical to the sequence of SEQ ID NO: 1; (2) a second composition comprising one or more oligonucleotide, wherein the one or more oligonucleotide comprises a sequence that is at least 90% identical to the full length of an oligonucleotide sequence selected from any one of SEQ ID NOs: 2-6; (3) a third composition comprising a real-time PCR buffer solution, dNTP, MgCl₂, and a DNA polymerase; and (3) instructions for performing any one of the methods described herein. Any suitable real-time PCR buffer solution can be used in the kits described herein.

In some embodiments, the kit further includes a high level control, a low level control, and a negative quality control. In some embodiments, the kit includes dNTPs and other necessary components for performing a multiplex quantitative real-time PCR reaction. The necessary components are known in the art.

### EXAMPLES

The invention is further described in the following examples, which do not limit the scope of the invention described in the claims.

### EXAMPLE 1: Primers, Probes and Kits for the Detection of cfDNA

This example describes a cell-free DNA detection kit that is a duplex fluorescence PCR assay intended for the quantitative detection of extracellular DNA (cfDNA) in human plasma from SARS-COV-2 confirmed patients as an aid for the assessment of disease severity which results from organ or tissue damage that can develop from the SARS-COV-2 infection.

The cell-free DNA kit is to be used with the QIAamp DNA Blood Mini Kit (QIAGEN, catalog #51104 or 51106) and the following multiplex quantitative real-time PCR Instruments and software:
Applied Biosystems™ 7500 Real-Time PCR Instrument System with software V1.4.1 or above
Applied Biosystems™ 7500 Fast Real-Time PCR Instrument System with software V1.4.1 or above
Roche Cobas z 480 Software version 1.5.0 or above.

TABLE 1
Primer and Probe Sequences and Modifications

| | | 5′ | 3′ | SEQ |
|---|---|---|---|---|
| | Sequence | Modifi- | Modifi- | ID |
| Name | (5′-3′) | cation | cation | NO |
| Human β-actin | GGACCTGA | / | / | 2 |
| Gene Forward | CTGACTAC | | | |
| Primer | CTCATGAA | | | |
| Human β-actin | CTTAATGT | / | / | 3 |
| Gene Reverse | CACGCACG | | | |
| Primer | ATTTCC | | | |
| Human β-actin | CACCGAGC | JOE | BHQ1 | 5 |
| Gene Probe | GCGGCTAC | | | |
| | AGCTTC | | | |
| Internal | AAACAGCT | / | / | 4 |
| Standard | ATGACCAT | | | |
| Oligonucleotide | GATTACGA | | | |
| Forward Primer | A | | | |
| Internal | CTTAATGT | / | / | 3 |
| Standard | CACGCACG | | | |
| Oligonucleotide | ATTTCC | | | |
| Reverse Primer | | | | |
| Internal | CCACCAAC | FAM | BHQ1 | 6 |
| Standard | CGCTCCCG | | | |
| Oligonucleotide | TAATCTCT | | | |
| Probe | AG | | | |

### Device Description and Test Principle

### 1) Product Overview/Test Principle:

The example cell-free DNA kit is a duplex fluorescence PCR assay which incorporates a novel technology with an internal standard for the quantitative detection of circulating cell-free DNA in plasma. Variation in cell-free DNA levels could sensitively provide a signal indicating the damage of tissues or organs and its severity. The combination of the unique design of primers and probes and the internal parameters solved the issues in traditional quantitative detection for cell-free DNA regarding poor accuracy and stability. Through the real-time dynamic monitoring of abnormal cell death, the quantitative measurement of cell-free DNA could play a crucial role in the assessment of COVID-19 patient's condition, treatment efficacy, and disease progression. The cell-free DNA kit can assist doctors in deciding which patients should be treated more aggressively and to help manage limited medical resource more efficiently.

The cell-free DNA kit is designed to target the specific DNA sequence of human β-actin (NCBI Accession No.: NG_007992.1), a housekeeping gene, for accurate cfDNA quantification (DNA primer/probe sequences (Chinese Patent Publication No. CN 106399536 A, the entire content of which is incorporated herein by reference). By providing a measurable indication of tissue damage caused by the COVID-19 complication, the cell-free DNA kit can provide crucial information to healthcare providers as to when a patient is more likely to experience severe disease which could aid in treatment and supportive care decisions. The cell-free DNA detection kit could thus help to improve the cure rate and lower the mortality rate of COVID-19.

The cell-free DNA quantitative detection kit is performed on standard laboratory PCR equipment and the concentration of cell-free DNA is calculated from the instrument output:

$$cfDNA concentration ( ng / mL ) = 84 \; 6.15 \times {2}^{( Ct , S - Ct , T )} \qquad ( II )$$

Where Ct,S is the Ct of internal standard and Ct,T is the Ct of target gene (β-actin).

The principal of the cell-free DNA concentration equation in shown below:

$${T}_{0} = {S}_{0} \times \frac{{( 1 + {E}_{S} )}^{Ct , S}}{{( 1 + {E}_{T} )}^{Ct , T}} \qquad ( I )$$

wherein:
T₀ is the concentration by copy number (copies/mL) of the cfDNA (e.g., human β-actin gene);
S₀ is the concentration by copy number (copies/mL) of the internal standard oligonucleotide;
E_{T} is the PCR amplification efficiency of the cfDNA (e.g., human β-actin gene);
E_{S} is the PCR amplification efficiency of the internal standard oligonucleotide;
Ct,T is the Ct (cycle threshold) value for the cfDNA (e.g., human β-actin gene);
Ct,S is the Ct (cycle threshold) value for the internal standard oligonucleotide.

The concentration (ng/ml) is calculated by using 3.3 pg of single-copy human genomic DNA as a conversion factor (see, e.g., Chen D, Pan S, Xie E, et al. Development and Evaluation of a Duplex Real-Time PCR Assay with a Novel Internal Standard for Precise Quantification of Plasma DNA. Ann Lab Med. 2017, 37:18-27).

### 2) Description of Test Steps:

### A. Sample Preparation

Sample collection: Draw 2 mL peripheral venous blood into an EDTA-K2 anticoagulation vacuum blood collection tube (purple).

### Plasma Isolation

### Step 1:

Equilibrate all samples to room temperature.
Centrifuge the 2 mL blood sample at 1,600 g for 10 minutes. Pipette 400 μL of the upper layer plasma carefully into a 1.5 mL DNase-free microcentrifuge tube.

### Step 2:

Centrifuge the resulted 400 μL plasma from “step 1” at 16,000 g for 10 minutes at 4° C., then pipette 195 μL of the upper layer plasma into a new 1.5 mL DNase-free microcentrifuge tube. This 195 μL is the isolated sample plasma.

### Addition of Internal Standard (IS)

### Step 1:

Thaw Reagent C from the kit at room temperature.

### Step 2:

Vortex for 10 seconds followed by performing a quick spin. Add 5 μL of Reagent C into each of three different tubes containing: 195 μL plasma sample (from the sample preparation step), 195 μL High Level Control (HC, Reagent D) and 195 μL Low Level Control (LC, Reagent E), respectively. Mark them separately and mix well for further operation.

### No Template Control (NTC, Reagent F)

Reagent F is the negative control (no template control) containing DNase-free water. Pipette 200 μL of the NTC into a new 1.5 mL DNase-free microcentrifuge tube.

### Nucleic Acid Extraction (Manual)

QIAamp DNA Blood Mini Kit (QIAGEN, catalog #51104/51106) has been validated and is recommended for the use of nucleic acid extraction. 8 μL from each of the samples and controls after the extraction will be used for running the test.

B. PCR reagent preparation (Master Mix)
Thaw Reagent A and Reagent B to room temperature. Then mix by pulse-vortexing for 10 seconds, followed by a quick spin.
Take out the required number, N*, PCR reaction tube(s)
Aliquot 17 μL PCR mixture into each of the PCR reaction tubes (or wells) as per Table 2.
The total volume of PCR-Mix is N×17 μL.
After preparation, the Reagent A and B should immediately be tightly covered and stored in the dark at −20±5° C.
Note: N=number of samples to be tested+1 (high level control)+1 (low level control)+1 (negative control)+1 (sampling error).

TABLE 2
PCR Master Mixture Volume Calculations

Amount (μL)

| Reagent | Single Reaction | N Reactions |
|---|---|---|
| PCR Reaction Mix A | 16 | 16 × N |
| PCR Reaction Mix B | 1 | 1 × N |
| Total | 17 | 17 × N |

C. Sample addition
Add 8 μL of the samples yield from the extraction step into the above PCR reaction tubes/wells. Cap the tubes/well.
The total volume for each tube/well will be 25 L:
17 μL (PCR Mix)+8 μL (Sample extraction)=25 μL

### D. Instrument Channel Setting

### Software Setting

Applied Biosystems™ 7500 Real Time PCR System (software v 1.5 and above)

The manufacture's manual for general instruction is provided in the manufacture's website: thermofisher.com/order/catalog/product/4351106?SID=srch-srp-4351106 #/4351106?SID=srch-srp-4351106

Briefly:
1. New Experiment:→enter the name of this experiment.

Select sequentially 7500 (96 Wells)→Quantitation-Standard Curve→TaqManReagents→Standard.
2. Plate Setup→Define Targets and Samples →Define targets→Add New Target→Target information set up (Table 3)→Sample Name
3. Assign Targets and Sample-Click a well from View Plate Layout→activate all targets and tasks→select None for passive reference→Assign sample(s) to the selected wells
4. Channel Setting (Table 4)
5. Save→Start run.

TABLE 3
Target information

| Target Name | Reporter | Quencher | Color |
|---|---|---|---|
| Target 1 (IS*) | FAM | None | Color 1 |
| Target 2 (β-actin) | JOE | None | Color 2 |

*IS: Internal Standard (Reagent C)

TABLE 4
PCR Conditions

| | | | | | Data |
|---|---|---|---|---|---|
| Stage | Reps | Temp | Time | | Collection |
| Holding | 1 | 95° C. | 10 | min | |
| Stage | | | | | |
| Cycling | 40 | 94° C. | 30 | sec | |
| Stage | | 56° C. | 30 | sec | |
| | | 72° C. | 40 | sec | Yes |

4) Analysis
   1) Click Analysis.→Amplification Plot, under Plot Settings tab→select sequentially ΔRn vs Cycle (default)→log (default)→Target.
   2) Click Analysis Settings, in Ct Settings dialog box, click Edit Default Settings, deselect “automatic threshold” and “automatic baseline” and then set up threshold and baseline manually. Enter 3 in Baseline Start Cycle field, End Cycle value is set to the number which is less 1 or 2 cycles than the Ct value at the start of exponential amplification.
   3) Under options tab, select target (Reporter) to be adjusted, adjust the threshold value manually. The threshold values for FAM channel and JOE channel of the same sample must be the same. The threshold line intersects with the amplification curves of different channels of the same sample in the exponential amplification period.
   4) Ct values will be calculated after adjusting threshold. To review a Ct value of a sample, click the well. In the Target drop down, select the target for review.
      Roche Cobas® z 480 Real-Time PCR Instrument System (software V1.5.0)

The manufacture's manual for general instruction is provided in the manufacture's website: lifescience.roche.com/en_us/products/lightcycler14301-480-instrument-ii.html #documents

Briefly
1) Click New Experiment to set up experiment.
2) In the drop-down menu next to Detection Format, select Dual Color Hydrolysis Probe/UPL Probe for target information set up.

Click Customize and open Detection Format dialog box to set filter combination, select FAM and JOE.

In Reaction Volume field, enter 25 μL.

Filter selection for Cobas z 480: choose 465-510 for FAM; 540-580 for JOE (Table 5).

TABLE 5
Roche Cobas Set-up

Filter Combination Selection*

| Integration | Filter | Excitation | Emission |
|---|---|---|---|
| Time Mode | Combination | Filter | Filter |
| Dynamic | FAM | 465 | 510 |
| | JOE | 540 | 580 |

*Filter Combination Selection can be set by clicking Detection Formats on the Tools menu.

3) Designate individual program under Program Name and set temperature and time parameters for each program in the Program Temperature Targets panel below, referring to the steps, number of cycles, temperature, and duration (Table 6). Use (+) and (−) buttons to add or delete steps in the interface.

TABLE 6
PCR Conditions

| Program | | Analysis | Target | Acquisition | Hold |
|---|---|---|---|---|---|
| Name | Cycles | Mode | (° C.) | Mode | (hh:mm:ss) |
| 1 | 1 | None | 95 | None | 00:10:00 |
| 2 | 40 | Quanti- | 94 | None | 00:00:30 |
| | | fication | 56 | None | 00:00:30 |
| | | | 72 | Single | 00:00:40 |

Click Save As Template to save the program. The template can be used for future experiments by clicking Apply Template.

After editing subset and defining all sample names for this experiment, select Start Run to run the test.
4) Analysis
   1) Click Analysis, under Create New Analysis dialog box, select Abs Quant/Fit Points; In Create New Analysis dialog box, select the name of this experiment from Subset list, and click to confirm.
   2) In the Cycle Range tab, set First Cycle at 5 and Last Cycle at 40.
   3) In the Noise Band tab, click Filter Comb 540-580 or Filter Comb 465-510. In the Filter Combination interface, select FAM (456-510) and JOE (540-580) respectively to adjust the Noise Band (The Noise Band position shall be as low as possible when it intersects the amplification curve of samples in a smooth area). Observe and compare the Noise Band values of FAM and JOE channels, then select the larger number as the threshold.
Important: Threshold setting principle: The threshold values for FAM channel and JOE channel of the same sample must be the same.
   4) In the Analysis tab, change Threshold (Auto) to Threshold (Manual). Enter above obtained threshold in Threshold field, click Calculate below the window to obtain Cp values of corresponding channel (FAM or JOE) in the test, right-click to export data.
   5) Click Filter Comb 540-580 or Filter Comb 465-510 to change the channel.
3) Control Material(s) to be Used:

The cell-free DNA Kit contains three (3) vials of controls: Reagent D, E and F.

Reagents D and E contain two levels of concentration of the reference standard (β-actin gene NCBI: NG_007992.1). Concentrations* are lot specific and provided on the outer labeling for each vial. The derivation of the lot concentrations during the manufacturing process are as follows.
Reagent D (HC): Sample is contrived from the reference standard at a concentration of 300× LoD. The Internal Standard is added followed by the extraction process. The process is repeated ten times for each sample. The result of the ten samples is used for calculating the average and SD.

$$300 \times LoD + / - 3 \times SD$$

Reagent E (LC): Sample is contrived from the reference standard at a concentration of 20× LoD. The Internal Standard is added followed by the extraction process. The process us repeated ten times for each sample. The result of the ten samples is used for calculating the average and SD.

$$20 \times LoD + / - 3 \times SD$$

Reagent F is the No Template Control, as well as the negative control, containing the DNase-free H₂O to control for any contaminations which would affect the accuracy of the result.

### Interpretation of Results

All test controls should be examined prior to interpretation of patient results. If the controls are not valid, the patient results cannot be interpreted.

The concentration of cell free DNA is calculated as follows:

$$cfDNA concentration ( ng / mL ) = 84 \; 6.15 \times {2}^{( Ct , S - Ct , T )}$$

Note: Ct,S is the Ct value of internal standard, and Ct,T is the Ct value of human β-actin gene in the same PCR reaction tube under the same threshold conditions.
All clinical samples must exhibit fluorescence growth curves. If not, the sample is considered invalid. If residual sample is available then repeat from the extraction step; otherwise, resampling, pretreatment and testing.
Ct of internal standard must be >20 and ≤38. If not, rerun the sample from the Addition of Internal Standard (IS) step.
Ct of the NTC (reagent F) must be either no value, or ≥38. If not, contamination is suggested.
Reagent D (HC) and Reagent E (LC) must exhibit fluorescence growth curves and the concentration calculated from the derived FAM and JOE Ct values must fall within the range of the corresponding values provided for each lot per the vial labeling.
If the control values do not fall within the range providing on labeling, readjust the threshold. Note: the threshold for both FAM and JOE must be the same.
The threshold for both the JOE and FAM channel must be the same until moving to the next step. Once all controls have been confirmed as above, proceed with the analysis for cell free DNA.

Each of the LC, HC must be qualified according to the concentration range on the label. Manually adjust the threshold until the value is in the range. Note: The threshold for both the JOE and FAM channel must be the same until moving to the next step. Once all controls have been confirmed as above, proceed with the analysis for cell free DNA. Results are interpreted according to Table 7:

TABLE 7
Cell-free DNA Kit Results Interpretation

| Result | Interpretation* | Actions |
|---|---|---|
| ≤30 | Level 1: Within normal | considered low risk |
| | range | |
| >30 and ≤95 | Level 2: Non-Severe | mild and moderate level of |
| | illness | severity, considering more |
| | | frequent monitoring |
| >95 | Level 3: Severe | severe and critical level of |
| | illness | severity; immediate clinical |
| | | follow-up and monitoring |
| | | needed |

The severity of COVID-19 is grouped as severe (severe to critical) and non-severe (mild to moderate) according to the WHO “Clinical management of COVID-19: interim guidance”. Patients are grouped as severe when the respiratory rate >30 breaths/min, or Pa02/Fi02<300 mm Hg; and the opposite as non-severe. This method was also adopted by 5 NIH later for the same type of classification.

Components Included with the Test

TABLE 8
Components supplied with the test kit

| | Label | Quantity | Volume | |
|---|---|---|---|---|
| Name | Name | (vial) | (μL/vial) | Description |
| Reagent A | RA | 1 | 1,540 | Probes, Primers, dNTPs, |
| | | | | and Tris-HCI buffer |
| Reagent B | RB | 1 | 100 | HotStart Taq DNA |
| | | | | Polymerase |
| Reagent C | RC | 1 | 500 | Synthetic DNA fragment |
| Reagent D: | HC | 1 | 1,000 | High level of β -actin |
| High level | | | | gene fragment |
| control | | | | |
| Reagent E: | LC | 1 | 1,000 | Low level of β-actin |
| Low level | | | | gene fragment |
| control | | | | |
| Reagent F: | NC | 1 | 1,000 | DNase-free H₂O |
| Negative | | | | |
| control | | | | |

Components Required but not Included with the Test
Components required but not included with the test:
Refer to manufacturer's instructions for:
   Applied Biosystems 7500 and 7500 Fast
   Roche Cobas z 480
Extraction Kits
   QIAamp DNA Blood Mini Kit (QIAGEN, catalog #51104 or 51106).
Other Instruments and Consumables
   Vortex
   Centrifuge
   Pipette 10 μL, 200 UL, 1000 UL
   96-well microplate
   DNase-free tips
   Disposable gloves
   1.5 mL DNase-free microcentrifuge tubes
   PCR reaction tubes (suitable for Applied Biosystems 7500 or Roche Cobas z 480)

### Testing Capabilities

Dependent upon instrument parameters.

### Reagent Stability:

Cell-free DNA Kit stability has been demonstrated through real-time, in-use and shipping stability studies. Each of the studies is described in the section below. The acceptance criteria for each of the stability tests is as follows:
Linear range: 10 ng/ml-2400 ng/ml, regression coefficient r ≥0.980.
Accuracy: Absolute deviation does not exceed+0.5 logarithmic orders of magnitude.
Intra-batch precision: Coefficient of variation (CV, %) of the logarithm of the detected concentration is ≤5% if the reference standard is >30 ng/ml; ≤10% if the reference standard is ≤30 ng/mL.
Inter-batch precision: The CV % of logarithm of the detected concentration is ≤15%.
Limit of Quantitation (LoQ): 10 ng/mL, and CV % of logarithm of the detected concentration is ≤10%.
Limit of Detection (LoD): 1 ng/mL, among which: a. ≥95% detection rate for the β-actin gene; b. 100% detection rate for the internal standard.

### Real-Time Stability Plan:

A real-time stability study to determine the shelf life for the cell-free DNA Quantitative Detection Kit with kits randomly selected from three kit lots has been designed and carried out according to ‘In vitro diagnostic medical devices—Evaluation of stability of in vitro diagnostic reagents [EN ISO 23640:2015]” and “CLSI EP25-A”. Samples for testing are contrived at concentrations of 2400 ng/ml, 800 ng/ml and 300 ng/ml, 266.67 ng/ml, 88.89 ng/mL, 29.63 ng/mL, 20 ng/mL, 10 ng/mL, 9.88 ng/mL, and 1 ng/mL using a reference standard. For each time-point of testing, ten (10) sample replicates are tested along with positive and negative quality control samples. Kits are stored at −20±5° C. prior to testing. The testing is being conducted according to the following schedule: day 0, day 122, day 245, day 366, day 427. Day 0 test point is the first day of production date. cell-free DNA Quantitative Detection Kit testing is performed according to the instructions for use on the Roche Cobas z 480 PCR detection system (software v1.5.0) or Applied Biosystems 7500 Real Time PCR System (v2.0.6).

Based on current data up to day 427, the claimed shelf-life: 12 months.

### Performance Evaluation

### Limit of Bank (LoB)

a. Study Protocol

The LoB study was design based on CLSI EP17. Samples at the concentration of 300 ng/ml and 20 ng/ml were obtained by mixing the contrived plasma sample with the reference solution, respectively. All samples were treated with DNase before using. Measured cell-free DNA Quantitative Detection Kit with ten replicates per day for 3 days. The experiment was repeated with three lots of the kit to evaluate the LoB.

b. Conclusion

The results did not have any Ct value and, therefore, the LoB of the cell-free DNA kit is not applicable.

### Limit of Detection (LoD)

### Study Protocol

### Step 1: Tentative LoD Determination

Sample at the concentration of 1000 ng/ml was contrived by mixing contrived plasma samples with the reference solution. Initial LoD was determined by testing at the gradient dilution series included five replicates at every concentration of 0.1 ng/ml, 1.0 ng/mL, 10 ng/mL, 100 ng/mL, and 1000 ng/mL, respectively.

### Data Summary:

1000 ng/mL: 5/5 were detected.
100 ng/mL: 5/5 were detected.
10 ng/ml: 5/5 were detected.
1 ng/ml: 5/5 were detected.
0.1 ng/mL: 1/5 were detected.

### Step 2: LoD Confirmation

The LoD was confirmed by testing 20 replicates at three different concentrations of 1 ng/mL, 0.5 ng/mL, 0.25 ng/ml, respectively.

### Data Summary:

1 ng/ml: 20/20 were detected.
0.5 ng/ml: 10/20 were detected.
0.25 ng/mL: 5/20 were detected.

### Study Conclusion

The LoD of the cell-free DNA Quantitative Detection Kit was confirmed at 1 ng/mL.

### Limit of Quantitation (LoQ)

### Study Protocol

### Step 1: Tentative LoQ Determination

Sample with concentration of 1000 ng/mL was contrived by mixing the plasma samples with reference solution. LoQ was determined by testing at the gradient dilution series included five replicates at every concentration of 0.1 ng/ml, 1.0 ng/ml, 10 ng/mL, 100 ng/mL, and 1000 ng/ml, respectively.

a. Study Summary
5/5 replicates were detected at 1000 ng/mL. The coefficient of variation of logarithm of measured concentration was 1.22% (≤10%).
5/5 replicates were detected at 100 ng/mL. The coefficient of variation of logarithm of measured concentration was 1.36% (≤10%).
5/5 replicates were detected at 10 ng/mL. The coefficient of variation of logarithm of measured concentration was 9.92% (≤10%).
5/5 replicates were detected at 1 ng/mL. The coefficient of variation of logarithm of measured concentration was 153.59% (>10%).
1/5 replicates were detected at 0.1 ng/mL.

### Step 2: LoQ Confirmation

The LoQ was confirmed by testing 20 replicates at three different concentrations of 10 ng/mL, 5 ng/mL, 2.5 ng/mL, respectively.

b. Study Summary
20/20 replicates were detected at 10 ng/mL. The coefficient of variation of logarithm of measured concentration was 9.93% (≤10%).
20/20 replicates were detected at 5 ng/mL. The coefficient of variation of logarithm of measured concentration was 26.79%.
20/20 replicates were detected at 2.5 ng/mL. The coefficient of variation of logarithm of measured concentration was 42.74%.

Conclusion: The LoQ for the cell-free DNA quantitative detection kit was found to be 10 ng/mL.

### Precision

a. Study Protocol

Samples with concentration of 300 ng/ml and 20 ng/ml were contrived by mixing the plasma with reference solution. Precision was evaluated by the coefficient of variation (CV, %) of logarithm of measured concentration.

Intra-batch precision: 300 ng/mL and 20 ng/mL samples were detected with 10 replicates by the same reagent lot, respectively. The coefficient of variation (CV, %) of logarithm of 300 ng/ml sample was ≤5%; the coefficient of variation (CV, %) of logarithm of 20 ng/ml sample was ≤10%.

Inter-batch precision: 300 ng/mL and 20 ng/ml samples were detected with 10 replicates by three reagent lots, respectively. The coefficient of variation (CV, %) of logarithm of both measured concentrations were ≤15%.

Acceptance Criteria:
Intra-batch precision: Coefficient of variation (CV, %) of the logarithm of the detected concentration is ≤5% if the reference standard is >30 ng/ml; ≤10% if the reference standard is ≤30 ng/mL.
Inter-batch precision: The CV % of logarithm of the detected concentration is ≤15%.

### Study Summary

Conclusion: The precision of Cell-free DNA Kit met the acceptance criteria of intra-batch and inter-batch.

### Linearity

### Study Protocol

Sample at a concentration of 2400 ng/mL was contrived by mixing the plasma samples with reference synthetic β-actin DNA material. Linearity was evaluated by testing at the gradient dilution series included three replicates at 8 different concentrations of 2400 ng/mL, 800 ng/mL, 266.67 ng/mL, 88.89 ng/mL, 29.63 ng/mL, 9.88 ng/mL, 3.29 ng/ml and 1.1 ng/mL, respectively.

### Study Conclusion

The linearity range is 10 ng/ml-2400 ng/mL.

### Accuracy

### Study Protocol

Samples at concentrations of 300 ng/mL and 20 ng/mL were contrived by mixing the plasma samples with reference solution. Accuracy was evaluated by the absolute deviation between the logarithm of the measured concentration and the logarithm of the theoretical concentration. 300 ng/mL and 20 ng/mL samples were tested with 3 replicates from the same lot, respectively. The absolute deviation between the logarithm of the measured concentration and the logarithm of the theoretical concentration of both 300 ng/ml and 20 ng/mL should not exceed ±0.5 log orders of magnitude.

### Study Conclusion

The concentrations tested with the cell-free DNA kit did not exceed ±10.5 log orders of magnitude, passing the acceptance criteria for assay accuracy.

### Normal Range

To determine the range of cell-free DNA measured in normal (non-diseased) populations, peripheral venous blood samples were obtained from 213 apparently healthy individuals and analyzed with the cell-free DNA kit. The normal reference interval up to 30.65 ng/ml (95th percentile) was determined. The data is summarized in Table 9.

TABLE 9
Summary of Normal Reference Range Study

| | | cfDNA upper |
|---|---|---|
| Age Range | | 95% Interval |
| (years) | n | (ng/mL) |
| 30-80 | 213 | 30.65 |
| | (97 males; | |
| | 116 females) | |

Note:
Each laboratory should establish its own reference ranges to assure proper representation of specific populations.

### Cross-reactivity (Analytical Specificity):

Evaluation of analytical specificity of the Cell-free DNA kit was determined using both, in-silico analysis and wet testing against pathogenic organisms mainly found in the human respiratory tract (Table 10).

### In-silico Analysis:

BLASTn analysis queries of the cell-free DNA kit primers and probes (human β-actin primer/probe set and internal standard primer/probe set) were performed against public domain nucleotide sequences with the following database search parameters:
Mask low complexity regions=Yes
Expectation value=10
Match/Mismatch=Match 2 Mismatch-3
Gap Costs=Existence 5 Extension 2
Max number of hit sequence=250
Mask lower case=No
Mask low complexity regions=Yes
Number of threads=16
Filter out redundant results=No.

TABLE 10
In-silico Cross-Reactivity Analysis

| | | | | Internal |
|---|---|---|---|---|
| | | | β-actin | Standard |
| | | | (2 primers, | (2 primers, |
| | Microorganism | Ref. Seq | 1 probe) | 1 probe) |
| 1 | Human coronavirus | NC_002645.1 | F: 41.67% | F: 40.00% |
| | 229E | | R: 40.91% | R: 40.91% |
| | | | P: 36.36% | P: 34.62% |
| 2 | Human coronavirus | NC_006213.1 | F: 41.67% | F: 36.00% |
| | OC43 | | R: 50.00% | R: 50.00% |
| | | | P: 40.91% | P: 38.46% |
| 3 | Human coronavirus | NC_006577.2 | F: 37.50% | F: 40.00% |
| | HKU1 | | R: 40.91% | R: 40.91% |
| | | | P: 45.45% | P: 30.77% |
| 4 | Human coronavirus | NC_005831.2 | F: 37.50% | F: 36.00% |
| | NL63 | | R: 50.00% | R: 50.00% |
| | | | P: 40.91% | P: 34.62% |
| 5 | SARS-coronavirus | NC_004718.3 | F: 33.33% | F: 48.00% |
| | | | R: 50.00% | R: 50.00% |
| | | | P: 40.91% | P: 50.00% |
| 6 | MERS-coronavirus | NC_019843.3 | F: 50.00% | F: 48.00% |
| | | | R: 45.45% | R: 45.45% |
| | | | P: 36.36% | P: 34.62% |
| 7 | Human adenovirus 1 | AC_000017.1 | F: 37.50% | F: 41.67% |
| | | | R: 45.45% | R: 45.45% |
| | | | P: 45.45% | P: 34.62% |
| 8 | Human adenovirus 2 | AC_000007.1 | F: 37.50% | F: 40.00% |
| | | | R: 50.00% | R: 50.00% |
| | | | P: 45.45% | P: 34.62% |
| 9 | Human adenovirus 3 | DQ086466.1 | F: 41.67% | F: 36.00% |
| | | | R: 40.91% | R: 40.91% |
| | | | P: 40.91% | P: 42.31% |
| 10 | Human | NC_039199.1 | F: 37.50% | F: 32.00% |
| | Metapneumovirus | | R: 40.91% | R: 40.91% |
| | | | P: 36.36% | P: 30.77% |
| 11 | Human | NC_003461.1 | F: 41.67% | F: 44.00% |
| | parainfluenza | | R: 45.45% | R: 45.45% |
| | virus 1 | | P: 45.45% | P: 34.62% |
| 12 | Human | AB176531.1 | F: 37.50% | F: 36.00% |
| | parainfluenza | | R: 36.36% | R: 36.36% |
| | virus 2 | | P: 40.91% | P: 30.77% |
| 13 | Human | NC_001796.2 | F: 33.33% | F: 36.00% |
| | parainfluenza | | R: 45.45% | R: 45.45% |
| | virus 3 | | P: 31.82% | P: 38.46% |
| 14 | Human | NC_021928.1 | F: 37.50% | F: 40.00% |
| | parainfluenza | | R: 40.91% | R: 40.91% |
| | virus 4a | | P: 36.36% | P: 30.77% |
| 15 | Human | AB543337.1 | F: 33.33% | F: 36.00% |
| | parainfluenza | | R: 40.91% | R: 40.91% |
| | virus 4b | | P: 40.91% | P: 38.46% |
| 16 | Influenza A (H1N1) | GCF_000865725.1 | F: 33.33% | F: 40.00% |
| | | | R: 31.82% | R: 31.82% |
| | | | P: 36.36% | P: 26.92% |
| 17 | Influenza A (H5N1) | GCF_000864105.1 | F: 37.50% | F: 36.00% |
| | | | R: 36.36% | R: 36.36% |
| | | | P: 40.91% | P: 34.62% |
| 18 | Influenza B virus | NC_002204.1 | F: 29.17% | F: 36.00% |
| | | | R: 31.82% | R: 31.82% |
| | | | P: 36.36% | P: 26.92% |
| 19 | Human enterovirus B | NC_001472.1 | F: 37.50% | F: 32.00% |
| | | | R: 40.91% | R: 40.91% |
| | | | P: 36.36% | P: 34.62% |
| 20 | Respiratory | NC_001803.1 | F: 33.33% | F: 36.00% |
| | syncytial | | R: 45.45% | R: 45.45% |
| | virus | | P: 45.45% | P: 42.31% |
| 21 | Human rhinovirus C | NC_009996.1 | F: 33.33% | F: 36.00% |
| | | | R: 45.45% | R: 45.45% |
| | | | P: 40.91% | P: 34.62% |
| 22 | Chlamydia | NC_005043.1 | F: 45.83% | F: 48.00% |
| | pneumoniae | | R: 45.45% | R: 45.45% |
| | TW-183 | | P: 59.09% | P: 46.15% |
| 23 | Haemophilus | DM109591.1 | F: 45.83% | F: 48.00% |
| | influenzae | | R: 63.64% | R: 63.64% |
| | type B | | P: 72.73% | P: 50.00% |
| 24 | Legionella | NZ_LR134380.1 | F: 50.00% | F: 52.00% |
| | pneumophila | | R: 54.55% | R: 54.55% |
| | NCTC12273 | | P: 54.55% | P: 46.15% |
| 25 | Mycobacterium | NC_000962.3 | F: 54.17% | F: 48.00% |
| | tuberculosis | | R: 54.55% | R: 54.55% |
| | H37Rv | | P: 63.64% | P: 53.85% |
| 26 | Streptococcus | NZ_LN831051.1 | F: 50.00% | F: 68.00% |
| | pneumoniae | | R: 59.09% | R: 59.09% |
| | NCTC7465 | | P: 54.55% | P: 46.15% |
| 27 | Streptococcus | NZ_LN831034.1 | F: 50.00% | F: 68.00% |
| | pyogenes | | R: 50.00% | R: 50.00% |
| | NCTC8198 | | P: 54.55% | P: 42.31% |
| 28 | Bordetella | NC_018518.1 | F: 45.83% | F: 48.00% |
| | pertussis | | R: 59.09% | R: 59.09% |
| | 18323 | | P: 68.18% | P: 50.00% |
| 29 | Mycoplasma | NZ_CP010546.1 | F: 50.00% | F: 44.00% |
| | pneumoniae | | R: 54.55% | R: 54.55% |
| | FH | | P: 45.45% | P: 53.85% |
| 30 | Pneumocystis | GCF_001477535.1 | F: 50.00% | F: 52.00% |
| | jirovecii | | R: 59.09% | R: 59.09% |
| | RU7 | | P: 54.55% | P: 53.85% |
| 31 | Candida | Database (BLAST | F: 54.17% | F: 68.00% |
| | albicans | www.candidagenome.org) | R: 72.73% | R: 72.73% |
| | | | P: No hits | P: No hits |
| | | | found | found |
| 32 | Staphylococcus | NZ_CP035288.1 | F: 45.83% | F: 48.00% |
| | epidermidis ATCC | | R: 50.00% | R: 50.00% |
| | 14990 | | P: 50.00% | P: 42.31% |
| 33 | Streptococcus | NZ_LR134274.1 | F: 58.33% | F: 56.00% |
| | salivarius | | R: 63.64% | R: 63.64% |
| | NCTC8618 | | P: 50.00% | P: 46.15% |
| 34 | Staphylococcus | NC_007795.1 | F: 45.83% | F: 56.00% |
| | aureus | | R: 59.09% | R: 59.09% |
| | NCTC 8325 | | P: 50.00% | P: 42.31% |

### Conclusion:

The in-silico cross-reactivity analysis above showed that the homology between any one of the primers/probes of the test and any sequence present from the above analyzed microorganisms is less than 80%. Therefore, neither cross reactivity, nor microbial interference is expected with the microorganisms analyzed.

### Cross Reactivity: Wet Testing

For the further evaluation of the potential for cross-reactivity of the Cell-free DNA Quantitative Detection Kit target sequences, wet-testing was performed.

A total of three replicates were tested for each potential cross-reactant. No unexpected cross-reactivity was observed for the organisms and viruses listed. The results can be seen in the table below (Table 11).

TABLE 11
Wet-testing cross-reactivity of the Cell-free DNA Kit

| | | Results internal |
|---|---|---|
| | Results human | standard |
| | β-actin Gene | Sequences |
| Organism | (#detected/tested) | (#detected/tested) |
| SARS-CoV-2 | Not detected (0/3) | Not detected (0/3) |
| Human coronavirus | Not detected (0/3) | Not detected (0/3) |
| 229E | | |
| Human coronavirus | Not detected (0/3) | Not detected (0/3) |
| OC43 | | |
| Human coronavirus | Not detected (0/3) | Not detected (0/3) |
| HKUI | | |
| Human coronavirus | Not detected (0/3) | Not detected (0/3) |
| NL63 | | |
| SARS-coronavirus | Not detected (0/3) | Not detected (0/3) |
| MERS-coronavirus | Not detected (0/3) | Not detected (0/3) |
| Influenza A virus | Not detected (0/3) | Not detected (0/3) |
| (H3N2) | | |
| Influenza A virus | Not detected (0/3) | Not detected (0/3) |
| (H1N1) | | |
| Parainfluenza virus 1 | Not detected (0/3) | Not detected (0/3) |
| Parainfluenza virus 2 | Not detected (0/3) | Not detected (0/3) |
| Rhinovirus 14 | Not detected (0/3) | Not detected (0/3) |
| Enterovirus 71 | Not detected (0/3) | Not detected (0/3) |
| Escherichia coli | Not detected (0/3) | Not detected (0/3) |

### Conclusion:

None of the pathogens tested in Table 11 yielded Ct results with the cell-free DNA kit described herein. In conclusion, there was no cross reactivity observed with the cell-free DNA kit described herein and the tested pathogens.

### EXAMPLE 2: Modification of cfDNA Detection Methods

### Modification of PCR Reaction Systems

The comparison of a previous PCR reaction system and a modified PCR reaction system is shown in Table 12.

TABLE 12
Comparison of PCR Reaction Systems

| | Previous Method | | | Modified Method | | |
|---|---|---|---|---|---|---|
| | Amount added | | | Amount added | | |
| | to 25 μL | Final | | to 25 μl | Final | |
| Component | (μL) | Concentration | | (μL) | Concentration | |
| 10× Real Time | 2.5 | 1× | | 2.5 | 1× | |
| PCR buffer | | | | | | |
| dNTPs | 4 | 0.4 | mM | 4 | 0.4 | mM |
| MgCl₂ | 6 | 6 | mM | 6 | 6 | mM |
| Forward Primer 1 | 0.1 | 200 | nM | 0.1 | 200 | nM |
| Forward Primer 2 | 0.1 | 200 | nM | 0.1 | 200 | nM |
| Reverse Primer | 0.1 | 700 | nM | 0.1 | 700 | nM |
| Probe 1 | 0.1 | 100 | nM | 0.1 | 100 | nM |
| Probe 2 | 0.1 | 100 | nM | 0.1 | 100 | nM |
| Hot start DNA | 0.2 | 1 U/25 | μL | 0.2 | 1 U/25 | μL |
| polymerase | | | | | | |
| DNA polymerase | / | / | | 0.8 | 3.2% | (v/v) |
| stabilizer | | | | | | |
| deionized water | 9.3 | / | | 3 | / | |
| Sample | 2.5 | 10% | (v/v) | 8 | 32% | (v/v) |

| | | | | |
|---|---|---|---|---|
| Total | 25 | / | 25 | / |

The comparison of a previous PCR reaction condition and a modified PCR reaction condition is shown in Table 13.

TABLE 13
Comparison of PCR Reaction Conditions

| | Previous Method | | | | | Modified Method | | | | |
|---|---|---|---|---|---|---|---|---|---|---|
| | | | | Cycle | | | | | Cycle | |
| Step | Temperature | Time | | Number | | Temperature | Time | | Number | |
| Pre- | 95° C. | 5 | min | 1 | Cycle | 95° C. | 10 | min | 1 | Cycle |
| heating | | | | | | | | | | |
| Cycles | 94° C. | 30 | s | 40 | Cycles | 94° C. | 30 | s | 40 | Cycles |

| | | | | | |
|---|---|---|---|---|---|
| 55° C. | 30 | s | 56° C. | 30 | s |
| 72° C. | 40 | s | 72° C. | 40 | s |

### Limit of Detection

The limit of detection using the modified method is shown in Table 14

TABLE 14
Limit of Detection Using the Modified Method

| | | | | | Internal |
|---|---|---|---|---|---|
| | | | | β-actin | Standard |
| Sample | s (Ct | t (Ct | cfDNA | detection | detection |
| Name | of FAM) | of JOE) | (ng/mL) | rate | rate |
| 10 ng/mL | 27.64 | 34.14 | 9.35 | 100% | 100% |
| | 27.71 | 34.24 | 9.16 | (20/20) | (20/20) |
| | 27.85 | 34.09 | 11.19 | | |
| | 27.88 | 33.64 | 15.61 | | |
| | 27.86 | 34.11 | 11.12 | | |
| | 27.9 | 33.81 | 14.07 | | |
| | 28.1 | 33.98 | 14.37 | | |
| | 27.89 | 33.39 | 18.70 | | |
| | 27.55 | 34.12 | 8.91 | | |
| | 27.65 | 33.42 | 15.51 | | |
| | 27.94 | 34.36 | 9.88 | | |
| | 27.62 | 33.56 | 13.78 | | |
| | 27.46 | 33.33 | 14.47 | | |
| | 27.84 | 33.81 | 13.50 | | |
| | 27.58 | 33.99 | 9.95 | | |
| | 28.08 | 34.09 | 13.13 | | |
| | 27.88 | 33.99 | 12.25 | | |
| | 27.8 | 33.6 | 15.19 | | |
| | 27.85 | 34.6 | 7.86 | | |
| | 27.89 | 34.1 | 11.43 | | |
| 1 ng/mL | 25.67 | 35.09 | 1.24 | 100% | 100% |
| | 25.74 | 34.46 | 2.01 | (20/20) | (20/20) |
| | 25.8 | 34.94 | 1.50 | | |
| | 25.88 | 35.83 | 0.86 | | |
| | 26.08 | 35.59 | 1.16 | | |
| | 26.09 | 35.6 | 1.16 | | |
| | 25.97 | 34.49 | 2.31 | | |
| | 26.13 | 35.74 | 1.08 | | |
| | 26.16 | 35.72 | 1.12 | | |
| | 25.93 | 36.65 | 0.50 | | |
| | 25.87 | 35.08 | 1.43 | | |
| | 26.11 | 35.55 | 1.22 | | |
| | 26.11 | 36.55 | 0.61 | | |
| | 26.22 | 34.39 | 2.94 | | |
| | 26.16 | 35.71 | 1.13 | | |
| | 26.17 | 35.97 | 0.95 | | |
| | 26.09 | 34.81 | 2.01 | | |
| | 26.11 | 34.48 | 2.56 | | |
| | 25.95 | 36.1 | 0.74 | | |
| | 26.11 | 34.41 | 2.68 | | |
| 0.1 ng/mL | 26.04 | NA | NA | 65% | 100% |
| | 26.02 | 38.16 | 0.19 | (13/20) | (20/20) |
| | 25.98 | 38.07 | 0.19 | | |
| | 26.11 | 37.6 | 0.29 | | |
| | 25.56 | NA | NA | | |
| | 25.61 | NA | NA | | |
| | 25.56 | 37.51 | 0.21 | | |
| | 25.53 | 37.62 | 0.19 | | |
| | 25.71 | 36.29 | 0.55 | | |
| | 25.58 | 37.62 | 0.20 | | |
| | 25.6 | 37.73 | 0.19 | | |
| | 25.52 | 37.5 | 0.21 | | |
| | 25.71 | 36.57 | 0.46 | | |
| | 25.08 | 36.46 | 0.32 | | |
| | 25.53 | NA | NA | | |
| | 25.64 | NA | NA | | |
| | 25.6 | NA | NA | | |
| | 25.72 | NA | NA | | |
| | 25.68 | 35.6 | 0.87 | | |
| | 25.87 | 37.96 | 0.19 | | |

The detection rate for 10 ng/ml β-actin is 100% (20/20), and the detection rate for the internal standard oligonucleotide is 100% (20/20). The detection rate for 1 ng/ml β-actin is 100% (20/20), and the detection rate for the internal standard oligonucleotide is 100% (20/20). The detection rate for 0.1 ng/mL β-actin is 65% (13/20), and the detection rate for the internal standard oligonucleotide is 100% (20/20). Therefore, the limit of detection for the modified method is 1 ng/ml.

### Limit of Quantification

TABLE 15
Limit of Quantification using the Modified Method

| | | s | t | | | | | Coefficient |
|---|---|---|---|---|---|---|---|---|
| Sample | | (Ct of | (Ct of | cfDNA | Log | | | of |
| Name | | FAM) | JOE) | (ng/mL) | (concentration) | Mean | SD | Variation |
| 10 | ng/mL | 27.64 | 34.14 | 9.35 | 0.97 | 1.09 | 0.10 | 8.98% |
| | | 27.71 | 34.24 | 9.16 | 0.96 | | | |
| | | 27.85 | 34.09 | 11.19 | 1.05 | | | |
| | | 27.88 | 33.64 | 15.61 | 1.19 | | | |
| | | 27.86 | 34.11 | 11.12 | 1.05 | | | |
| | | 27.9 | 33.81 | 14.07 | 1.15 | | | |
| | | 28.1 | 33.98 | 14.37 | 1.16 | | | |
| | | 27.89 | 33.39 | 18.70 | 1.27 | | | |
| | | 27.55 | 34.12 | 8.91 | 0.95 | | | |
| | | 27.65 | 33.42 | 15.51 | 1.19 | | | |
| | | 27.94 | 34.36 | 9.88 | 0.99 | | | |
| | | 27.62 | 33.56 | 13.78 | 1.14 | | | |
| | | 27.46 | 33.33 | 14.47 | 1.16 | | | |
| | | 27.84 | 33.81 | 13.50 | 1.13 | | | |
| | | 27.58 | 33.99 | 9.95 | 1.00 | | | |
| | | 28.08 | 34.09 | 13.13 | 1.12 | | | |
| | | 27.88 | 33.99 | 12.25 | 1.09 | | | |
| | | 27.8 | 33.6 | 15.19 | 1.18 | | | |
| | | 27.85 | 34.6 | 7.86 | 0.90 | | | |
| | | 27.89 | 34.1 | 11.43 | 1.06 | | | |
| 1 | ng/mL | 25.67 | 35.09 | 1.24 | 0.09 | 0.12 | 0.21 | 179.20% |
| | | 25.74 | 34.46 | 2.01 | 0.30 | | | |
| | | 25.8 | 34.94 | 1.50 | 0.18 | | | |
| | | 25.88 | 35.83 | 0.86 | −0.07 | | | |
| | | 26.08 | 35.59 | 1.16 | 0.06 | | | |
| | | 26.09 | 35.6 | 1.16 | 0.06 | | | |
| | | 25.97 | 34.49 | 2.31 | 0.36 | | | |
| | | 26.13 | 35.74 | 1.08 | 0.03 | | | |
| | | 26.16 | 35.72 | 1.12 | 0.05 | | | |
| | | 25.93 | 36.65 | 0.50 | −0.30 | | | |
| | | 25.87 | 35.08 | 1.43 | 0.15 | | | |
| | | 26.11 | 35.55 | 1.22 | 0.09 | | | |
| | | 26.11 | 36.55 | 0.61 | −0.22 | | | |
| | | 26.22 | 34.39 | 2.94 | 0.47 | | | |
| | | 26.16 | 35.71 | 1.13 | 0.05 | | | |
| | | 26.17 | 35.97 | 0.95 | −0.02 | | | |
| | | 26.09 | 34.81 | 2.01 | 0.30 | | | |
| | | 26.11 | 34.48 | 2.56 | 0.41 | | | |
| | | 25.95 | 36.1 | 0.74 | −0.13 | | | |
| | | 26.11 | 34.41 | 2.68 | 0.43 | | | |

The coefficient of variation of Log concentration (CV, %) for the 10 ng/ml group is 8.98%; and the coefficient of variation of Log concentration (CV, %) for the 10 ng/ml group is 179.20%. Therefore, the limit of quantification for the modified method is 10 ng/mL.

### Sample Linearity

Sample linearity is determined by linear regression of mean log concentration Yi and theoretical Log Xi, and the linear coefficient is r. For a detection kit, the acceptable r is |r|≥0.980.

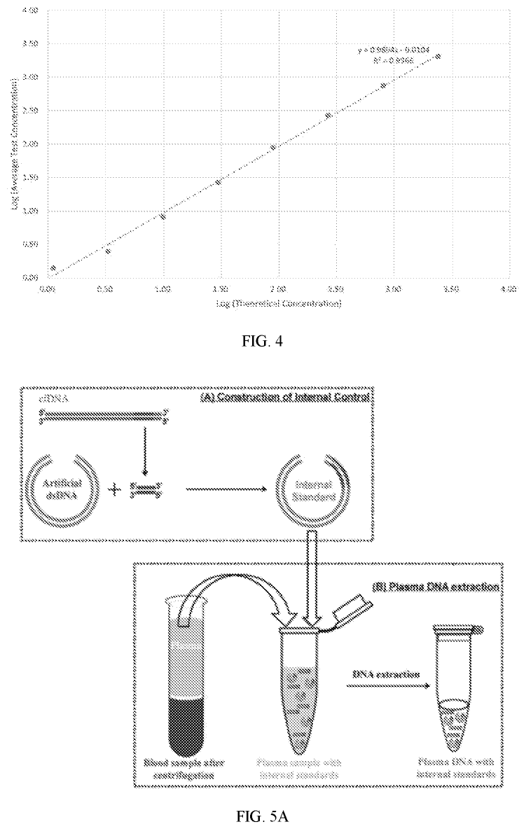

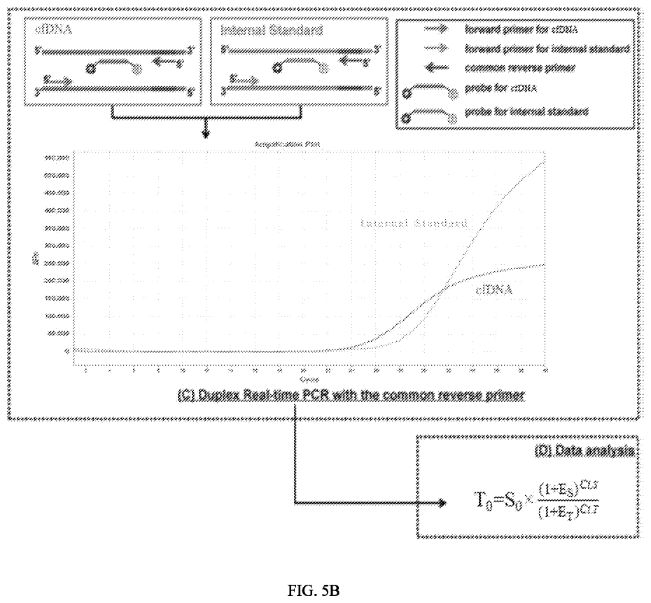

As shown in FIG. 4, r2 is 0.9966 for the 1.1-2400 ng/ml samples. |r|=0.9983 >0.9800. In addition, combined with the results of LoQ experiment, the cfDNA detection method has a sample linearity range of 10 ˜ 2400 ng/ml.

### Precision

As shown in Tables 16, the coefficient of variation (CV, %) of logarithm of 300 ng/ml was 2.83%, the coefficient of variation (CV, %) of logarithm of 20 ng/ml was 2.76%.

Therefore, the precision of modified method of cfDNA quantitative detection technology is qualified, and it is superior to the previous methods of cfDNA quantitative detection technology (e.g., the method described in Chinese Patent Publication No. CN 106399536 A) in terms of low concentration precision.

TABLE 16
Precision of the Modified Detection Method

| | | s | t | | | | | Coefficient |
|---|---|---|---|---|---|---|---|---|
| Sample | | (Ct of | (Ct of | cfDNA | Log | | | of |
| Name | | FAM) | JOE) | (ng/mL) | (concentration) | Mean | SD | Variation |
| 300 | ng/mL | 25.32 | 26.51 | 370.87 | 2.57 | 2.48 | 0.07 | 2.83% |
| | | 24.78 | 26.44 | 267.75 | 2.43 | | | |
| | | 24.87 | 26.5 | 273.38 | 2.44 | | | |
| | | 24.95 | 26.51 | 286.97 | 2.46 | | | |
| | | 25.2 | 26.56 | 329.65 | 2.52 | | | |
| | | 24.42 | 26.47 | 204.33 | 2.31 | | | |
| | | 25.11 | 26.45 | 334.25 | 2.52 | | | |
| | | 25.23 | 26.57 | 334.25 | 2.52 | | | |
| | | 25.12 | 26.63 | 297.09 | 2.47 | | | |
| | | 25.22 | 26.56 | 334.25 | 2.52 | | | |
| 20 | ng/mL | 25.7 | 30.77 | 25.19 | 1.40 | 1.43 | 0.04 | 2.76% |
| | | 25.81 | 30.72 | 28.14 | 1.45 | | | |
| | | 25.77 | 30.74 | 27.00 | 1.43 | | | |
| | | 25.71 | 30.76 | 25.54 | 1.41 | | | |
| | | 25.61 | 30.65 | 25.72 | 1.41 | | | |
| | | 25.58 | 30.64 | 25.37 | 1.40 | | | |
| | | 25.57 | 30.4 | 29.75 | 1.47 | | | |
| | | 25.61 | 30.3 | 32.78 | 1.52 | | | |
| | | 25.57 | 30.51 | 27.57 | 1.44 | | | |
| | | 25.7 | 30.87 | 23.50 | 1.37 | | | |

### Accuracy

The accuracy of the modified detection method is shown in Table 17.

For the 300 ng/ml sample, the absolute deviation range is about −0.08 ˜−0.01 and 5 does not exceed ±0.5 log orders of magnitude.

For the 20 ng/ml sample, the absolute deviation range is about 0.09 ˜ 0.14 and does not exceed ±0.5 log orders of magnitude.

Therefore, the accuracy of modified method of cfDNA quantitative detection was qualified, and the detection accuracy of high-value enterprise reference materials and low-value enterprise reference materials are higher than that of the previous method of cfDNA quantitative detection technology (e.g., the method described in Chinese Patent Publication No. CN 106399536 A).

TABLE 17
Accuracy of the Modified Detection Method

| | | | | | | Theoretical | | |
|---|---|---|---|---|---|---|---|---|
| | | s | t | | | cfDNA | | |
| Sample | | (Ct of | (Ct of | cfDNA | Log | concentration | | Absolute |
| Name | | FAM) | JOE) | (ng/mL) | (concentration) | (ng/mL) | Log | Deviation |
| 300 | ng/mL | 24.6 | 26.12 | 295.04 | 2.47 | 300 | 2.48 | −0.01 |
| | | 24.28 | 26.05 | 248.10 | 2.39 | 300 | 2.48 | −0.08 |
| | | 24.38 | 26.11 | 255.07 | 2.41 | 300 | 2.48 | −0.07 |
| 20 | ng/mL | 25.26 | 30.35 | 24.84 | 1.40 | 20 | 1.30 | 0.09 |
| | | 25.36 | 30.31 | 27.37 | 1.44 | 20 | 1.30 | 0.14 |
| | | 25.35 | 30.33 | 26.81 | 1.43 | 20 | 1.30 | 0.13 |

### Reference Range

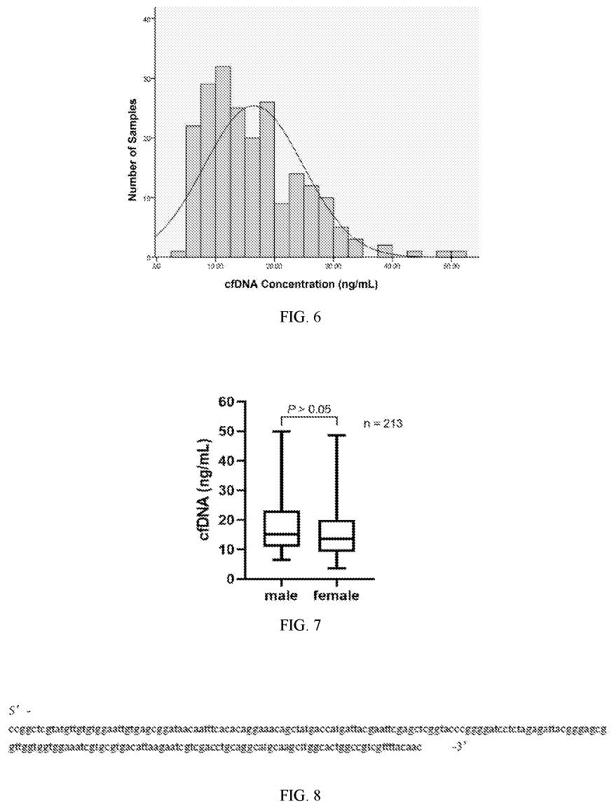

The reference range for the modified detection method is shown in FIGS. 6 and 7. Because of the optimization of the internal standard sequence and the primers and probes, the amplification for the internal standard is more stable and accurate. The reference range for the healthy sample group is therefore narrower than previous methods.

### EXAMPLE 3: Accurate Plasma DNA Quantification Reveals Disease Deterioration Timely in Hospitalized Patients with COVID-19

The coronavirus disease 2019 (COVID-19) outbreak has overwhelmed the world as one of the greatest threats (see, e.g., Woolf S H, Chapman D A, Lee J H. COVID-19 as the Leading Cause of Death in the United States. JAMA 2021; 325:123-4). The surge of infectors has been challenging healthcare systems worldwide. Effective patient triage and the following management are critical for optimizing medical resource allocation for those who expose to higher risks. Observations on a large population reported around 20% of patients developing severe and critical diseases with complications, such as acute respiratory distress syndrome (ARDS), sepsis, thromboembolism, and multi-organ failure (see, e.g., Wu Z, McGoogan J M. Characteristics of and Important Lessons From the Coronavirus Disease 2019 (COVID-19) Outbreak in China: Summary of a Report of 72314 Cases From the Chinese Center for Disease Control and Prevention. JAMA 2020; 323:1239-42). Early and timely assessment for the disease severity is crucial in providing optimized treatment and appropriate interventions, therefore improving the cure rate and lower mortality.

The WHO “Clinical management of COVID-19: interim guidance” (see, e.g., World Health Organization. Clinical management of COVID-19: interim guidance. website: who.int/publications/i/item/clinical-management-of-covid-19) adopts a set of definition based on pulmonary conditions and related complications for early triage and severity determination, yet limit mention in disease monitoring. The Acute Physiology and Chronic Health Evaluation (APACHE) II score and Sequential Organ Failure Assessment (SOFA) score are widely used to assess disease severity in general critical illnesses, which showed considerable prognostic efficacy for COVID-19 patients also having limited sensitivity or practicality issues (see, e.g., Zou X et al. Acute Physiology and Chronic Health Evaluation II Score as a Predictor of Hospital Mortality in Patients of Coronavirus Disease 2019. Crit Care Med 2020; 48: e657-65; Assaf D, et al. Utilization of machine-learning models to accurately predict the risk for critical COVID-19. Intern Emerg Med 2020; 15:1435-43; and Qu R, et al. C-reactive protein concentration as a risk predictor of mortality in intensive care unit: a multicenter, prospective, observational study. BMC Anesthesiol 2020; 20:292). Chest computed tomography (CT) imaging is considered to be effective for the early detection of lung involvement in COVID-19 (see, e.g., Zu Z Y, et al. Coronavirus Disease 2019 (COVID-19): A Perspective from China. Radiology 2020; 296: E15-25). However, its resolution is only applicable on millimeter level in imaging reading by human eyes causing inadequate sensibility in monitoring (see, e.g., Kwee T C, Kwee R M. Chest C T in COVID-19: What the Radiologist Needs to Know. Radiographics 2020; 40:1848-65). Recently, the SARS-COV-2 viral-antigens concentration in plasma was presented to be associated with disease progression, such as ICU admission and intubation rate, nonetheless these viral-antigens were not detectable consistently in all the COVID-19 positive patients (see, e.g., Ogata A F et al. Ultra-Sensitive Serial Profiling of SARS-COV-2 Antigens and Antibodies in Plasma to Understand Disease Progression in COVID-19 Patients with Severe Disease. Clin Chem 2020; 66:1562-72). Therefore, clinically available biomarkers that are objectively, rapidly, cost-effectively, quantitatively to convey reliable and real-time status of patients, are urgently needed not only for triage but also for monitoring.

A novel duplex real-time PCR assay with an internal standard, eliminating the preanalytical errors while increasing the precision and accuracy for autologous cfDNA quantification in plasma has been described in series of scenarios (see, e.g., Liu J-P, Zhang S-C, Pan S-Y. Value of dynamic plasma cell-free DNA monitoring in septic shock syndrome: A case report. World J Clin Cases 2020; 8:200-7; Chen D et al. 2011. The Clinical Significance of Plasma DNA Quantification for Quake Trauma Patients. In: Gahan P B, editor. Proceedings of the 6th international conference on circulating nucleic acids in plasma and serum. Hong Kong: Springer, Dordrecht. p. 171-82; Pan S et al. Can plasma DNA monitoring be employed in personalized chemotherapy for patients with advanced lung cancer? Biomed Pharmacother 2012; 66:131-7; Naumann D N, et al. Endotheliopathy is associated with higher levels of cell-free DNA following major trauma: A prospective observational study. PLoS One 2017; 12: e0189870; Chen D et al. Development and Evaluation of a Duplex Real-Time PCR Assay With a Novel Internal Standard for Precise Quantification of Plasma DNA. Ann Lab Med 2017; 37:18-27; Xia W-Y, et al. Liquid biopsy for non-invasive assessment of liver injury in hepatitis B patients. World J Gastroenterol 2019; 25:3985-95; and Wang H et al. Real-time monitoring efficiency and toxicity of chemotherapy in patients with advanced lung cancer. Clin Epigenetics 2015; 7:119). Thus, quantifying plasma DNA using the assay described herein is advantageous for the assessment of COVID-19 in monitoring patients.

### Methods

### Study Design and Participants

All the patients with confirmed COVID-19, excluding pregnancy and child, were enrolled and followed until discharge or 170 days of hospitalization. COVID-19 diagnoses were confirmed by positive RT-PCR assay for nasal and pharyngeal swab specimens. There was no available method to predetermine sample size, as patients were undergoing medical care.

### Outcomes

The severity of COVID-19 was classified by severe (severe to critical) and non-severe (mild to moderate) (see, e.g., Table 2 of World Health Organization, Clinical management of COVID-19: interim guidance, May 27, 2020). During monitoring, a series of un-overlapped 72-hour progressions of the disease was assessed from medical records of a 6-hour period compared against the period 72 hours later. We defined deterioration of progression by one or more of the followings: a) abnormally high or low body temperature; b) usage or increment of vasoactive, sedative, and analgesic agents; c) respiration supports upgrading; d) increasing of visible or occult bleeding; e) develop additional complications. The result of outcomes was given independently by three clinical experts with the consensus of at least two of them.

### Plasma DNA Quantification

The 2-mL blood sample anticoagulated with EDTA-K2 was used for plasma DNA quantification using the kit described herein. This test is a duplex fluorescence PCR assay using human β-actin gene as the amplification target and a synthetic DNA as the internal standard.

### Data Collection and Processing

The collected medical records of patients are not individually identifiable. Patient characteristics from medical records included the followings: demographic variables, clinical signs and symptoms, imaging results, laboratory findings, and medical history. Clinical signs and symptoms included: body temperature, systolic/diastolic blood pressure, heart rate, respiratory rate, vasoactive/sedative/analgesic agents administration and unconsciousness. Imaging results included abnormality of chest radiography or CT imaging. Laboratory findings included partial arterial oxygen pressure, oxygen saturation, white blood cell counts and differentiation, neutrophil to lymphocyte ratio (NLR), platelet counts, hematocrit, serum sodium and potassium, pH, total bilirubin, creatinine, and D-dimer levels. Medical history included: past operation, chronic obstructive pulmonary disease, liver cirrhosis, renal dialysis, immunodeficiency disease, cancer, chemotherapy, radiation, long term or high dose steroids.

The APACHE II and SOFA scores were calculated based on the worst value for each physiological variable within the past 24 hours of the visit timepoint and were averaged by two clinicians who were blinded to plasma DNA results.

### Statistical Analysis

The distribution of quantitative data was tested by Shapiro-Wilk normality test. Data with skewed distribution were shown as median with interquartile range (IQR). The correlation between variables was calculated using Spearman's rank coefficient. For a complete-case analysis, 15 missing values of D-dimer were handled using multiple imputation of predictive mean matching (PMM) from the whole dataset with nearest neighbors of 3. The introduced influence from imputation was evaluated through distribution comparison with no significant differences (P=0.685, see FIG. 13). Then the whole dataset was randomly divided into developing and validating subsets with a ratio of 3:1 in deterioration and non-deterioration, respectively. Eight variables, including disease severity, APACHE II score, SOFA score, plasma DNA, neutrophil counts, lymphocyte counts, NLR and D-dimer, were performed multivariate logistic regression with backward stepwise selection, removing terms with P >0.1 and adding those with P<0.05. Considering the multisampling of disease progression in a same patient, data in the above regression were deemed as clustered by patient's ID. Based on odds ratio to identify correlators for the prediction model's development, the estimate of model performance was determined on bootstrapping resampling, and the goodness-of-fit was assessed using Pearson x2 test.

Finally, the validating subsets were used to ascertain the discrimination performance by nomogram and its calibration curve. The decision curve analysis (DCA) was performed by calculating the net benefits of the nomogram for a range of threshold probabilities. All statistical analysis was performed using Stata/IC 15.0 (StataCorp LLC, USA) and R statistical software (version 4.0.3). Except as otherwise noted, two-tailed P<0.05 was considered to indicate a statistically significant difference.

### Results

### Characteristics of the Cohort

A total of 17 patients, with one exclusion due to hospital stay less than 72 hours, 6 females and 11 males, were included, aged from 27 to 83 years with a median age of 57 years (IQR: 50-72 years). Ten were admitted in intensive care unit (ICU) on the day of study entry, of which 7 underwent intratracheal intubation and 2 were treated with extracorporeal membrane oxygenation (ECMO). By the end of study, 15 patients were discharged with cured (median follow-up: 46 days, IQR: 20-59 days), 2 patients remained hospitalized, and no death reported. On the enrollment, patients with severe diseases were higher in APACHE II (P=0.002) and SOFA (P=0.001) scores in comparing to those with non-severe. And the age (P=0.29) and sex ratio (P >0.99) had no significant differences between them (Table 18).

TABLE 18
Baseline characteristics of patients with COVID-19

| | Mild or moderate | Severe or critical | |
|---|---|---|---|
| | (n = 9) | (n = 8) | P Value |
| Age, y | 63.5 (56.5-71.5) | 54 (50-72) | 0.289 |
| Sex | | | |
| Female | 3 | 3 | |
| Male | 6 | 5 | >0.99 |
| APACHE II score | 3 (2-6) | 17 (14.5-19.5) | 0.002 |
| SOFA score | 0 (0-1) | 9 (5-10) | 0.001 |
| ICU admission | 2 | 8 | 0.002 |
| Intratracheal intubation | 1 | 6 | 0.015 |
| ECMO treatment | 0 | 2 | 0.206 |
| CRRT* | 0 | 2 | 0.206 |
| Comorbidities | 5 | 5 | >0.99 |
| Hypertension | 4 | 5 | 0.637 |
| Cardiovascular disease | 1 | 3 | 0.294 |
| Diabetes | 3 | 2 | >0.99 |
| Chronic liver disease | 0 | 1 | 0.471 |

*CRRT: continuous renal replacement therapy

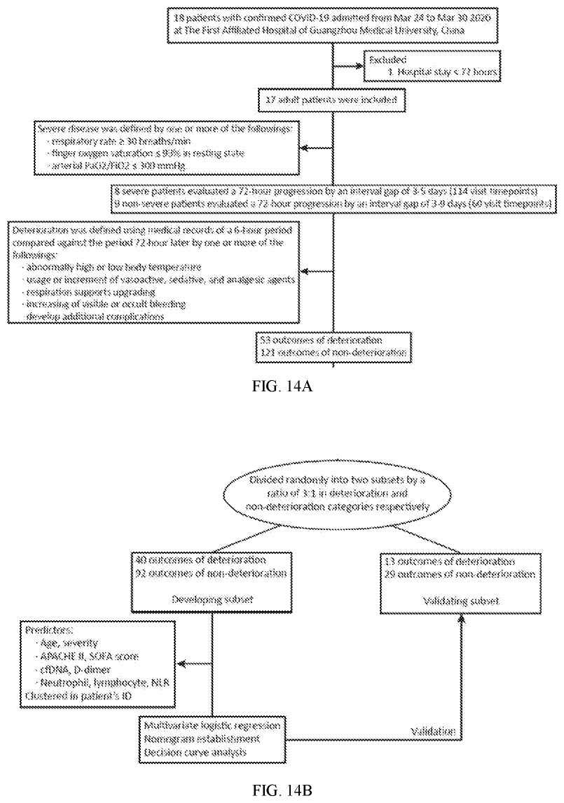

A total of 174 observation visits were determined 72-hour disease progression, of which 114 outcomes were determined from 8 patients categorized as severe on study entry with an interval visiting gap of 3-5 days (median: 3 days), and 60 outcomes from 9 non-severe with an interval gap of 3-9 days (median: 4 days). Then the whole outcome dataset was randomly divided into developing and validating subsets by a ratio of 3:1 in deterioration and non-deterioration, respectively, containing 40 outcomes of deterioration and 92 outcomes of non-deterioration in developing subset, and 13 of deterioration and 29 of non-deterioration in validating subset. The study workflow is provided in FIGS. 14A-B. There were no significant differences in candidate predictors between the two subsets (see Table 19).

TABLE 19
The candidate predictors between the developing and validating subsets

| | Developing (n = 132) | Validating (n = 42) | P Value |
|---|---|---|---|
| APACHE II score | 11 (7.5-18) | 10.5 (8-15) | 0.877 |
| SOFA score | 4 (2-7) | 5 (2-8) | 0.854 |
| Plasma DNA (ng/mL) | 136.2 (82.3-253.1) | 122.1 (67.2-218.4) | 0.425 |
| D-dimer (ng/ml) | 1392 (790-3322) | 1984 (797-3344) | 0.970 |
| Neutrophil count (×10⁹/L) | 4.92 (3.54-6.88) | 5.15 (3.47-6.97) | 0.981 |
| Lymphocyte count (×10⁹/L) | 1.23 (0.84-1.74) | 1.20 (0.79-1.72) | 0.981 |
| NLR* | 4.17 (2.78-6.12) | 3.92 (3.04-5.38) | 0.938 |

*NLR, neutrophil to lymphocyte ratio

### Comparison Between Severe and Non-Severe Status

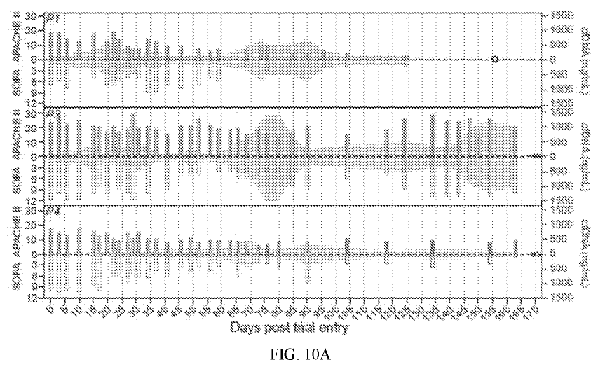

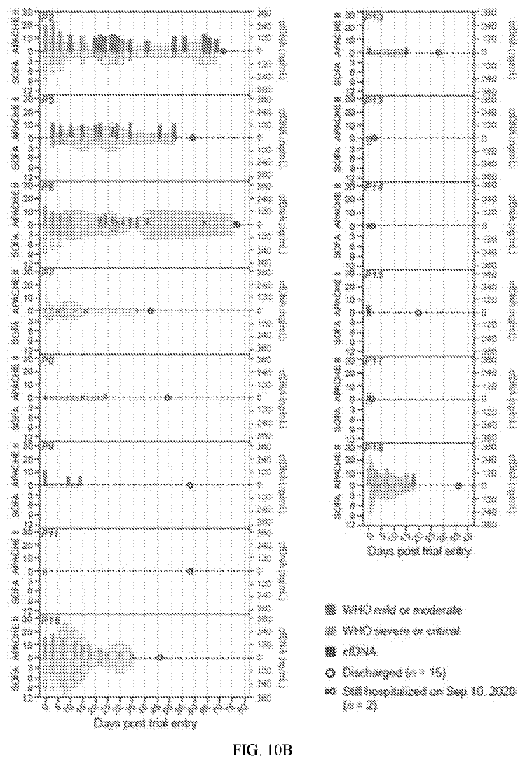

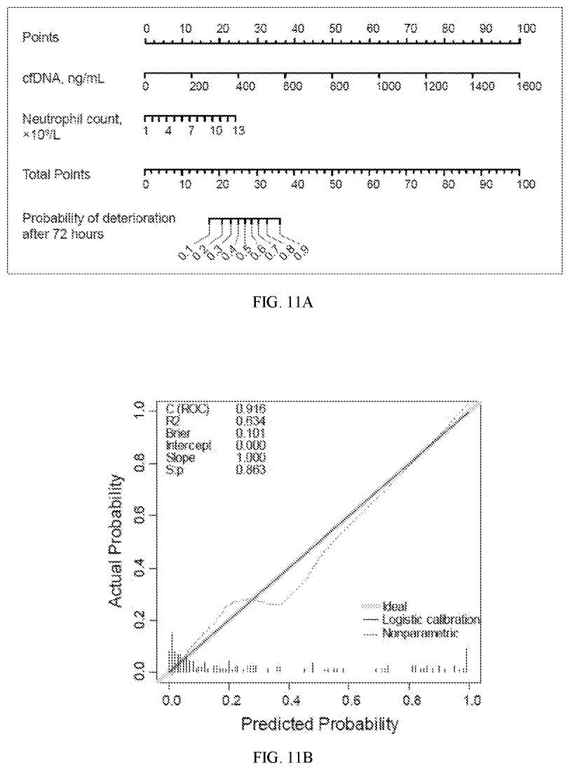

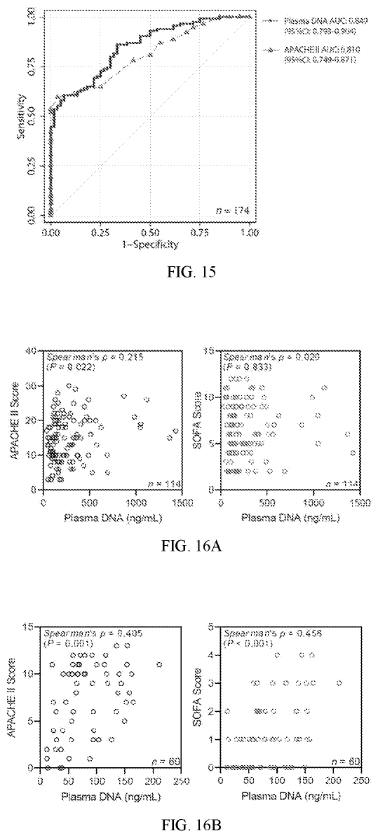

The comparison of severity scores and laboratory tests between severe and non-severe status during monitoring are presented in Table 20. Patients of severe status were definitely higher in APACHE II (P<0.001) and SOFA (P<0.001) scores, and more likely to have coagulopathy with a higher D-dimer level (P<0.001). Impaired immunity has been proved more commonly during severe status with a lower lymphocyte count, together with a higher level of neutrophil, achieved a significant higher NLR compared with those of non-severe status. The receiver operating characteristic (ROC) analysis showed that AUC of plasma DNA was 0.849 (95% CI: 0.793-0.904) with a sensitivity of 86.0% and specificity of 66.7% at the cut-off value of 95.02 ng/mL, which has no statistic difference with that of APACHE II score (AUC: 0.810, 95% CI: 0.749-0.871; plasma DNA vs. APACHE II: P=0.323; see FIG. 15). Plasma DNA levels had a similar pattern against the severity scores in different severity status, but did not dynamically parallel with severity scores as view from the longitudinal 5 monitoring (FIGS. 10A-B). The Spearman's correlation tests showed that plasma DNA had a rather weak coefficient (p) of 0.215 (P=0.022) with APACHE II score and did not correlate to SOFA score (P=0.833) in severe status, while in non-severe the coefficient was 0.405 (P =0.001) for APACHE II score and 0.458 (P<0.001) for SOFA score (FIGS. 16A-B), respectively, suggesting an additional role in disease severity assessment.

TABLE 20
Severity scores and laboratory tests between severe and non-severe status

| | Severe (n = 114) | Non-severe (n = 60) | P Value |
|---|---|---|---|
| APACHE II score | 15 (10-20) | 8.5 (4-10.5) | <0.001 |
| SOFA score | 6 (4-9) | 1 (0-2) | <0.001 |
| Plasma DNA (ng/ml) | 172.0 (109.5-345.9) | 74.3 (47.8-118.6) | <0.001 |
| D-dimer (ng/ml) | 2565 (1167-5854) | 708 (348-986) | <0.001 |
| Neutrophil count (×10⁹/L) | 5.34 (4.46-7.27) | 3.63 (2.86-5.40) | <0.001 |
| Lymphocyte count (×10⁹/L) | 0.99 (0.72-1.43) | 1.60 (1.20-2.32) | <0.001 |
| NLR* | 4.97 (3.74-6.81) | 2.36 (1.72-3.26) | <0.001 |

*NLR, neutrophil to lymphocyte ratio.

### Prediction Model Development and Validation

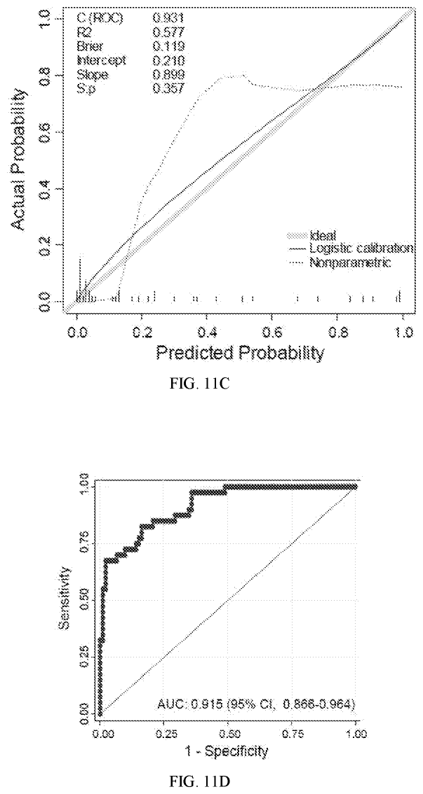

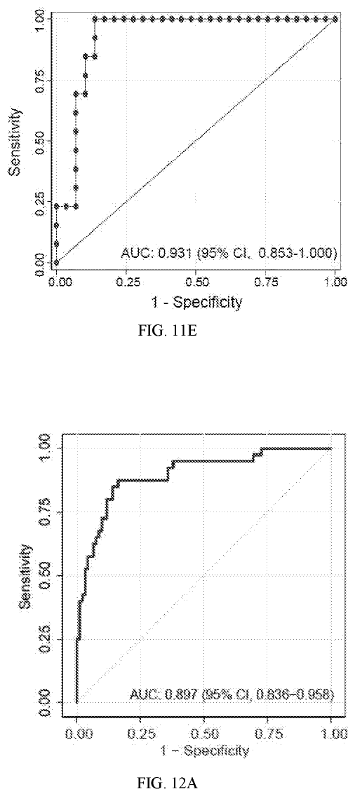

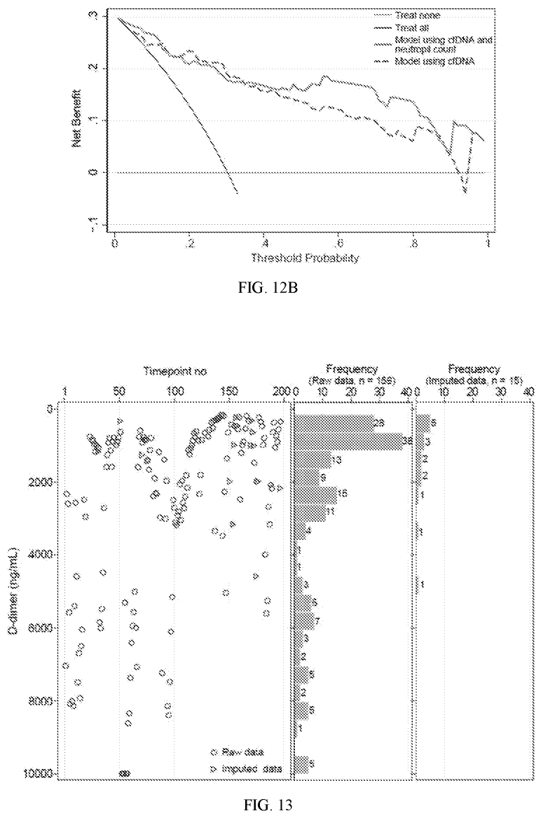

Baseline variables and laboratory tests that were considered clinically relevant were selected as candidate predictors, including WHO disease severity, APACHE II score, SOFA score, plasma DNA, D-dimer, neutrophil count, lymphocyte count and NLR. All the indices showed a significant correlation to disease deterioration in univariate analysis. Given the number of events available and high correlations (p >0.7) among predictors (Table 21), a backward stepwise multivariate logistic regression was applied using P >0.1 to remove variables, and clustered in patient's ID. After internal validation with bootstrapping 300 times, plasma DNA and neutrophil count were retained as independent predictors for deterioration in the developing dataset (Table 22). A nomogram incorporating these two predictors was then constructed (FIG. 11A). The nomogram's calibration curve (FIG. 11B) and a nonsignificant Pearson x2 statistic (P=0.987) showed good calibration in the developed model. The favorable calibration of the nomogram was confirmed with a nonsignificant Pearson x2 test statistic (P=0.670) in the validating dataset (FIG. 11C). The C-index of the nomogram in developing and validating subsets was 0.915 (95% CI: 0.866-0.964, FIG. 11D) and 0.931 (95% CI: 0.853-1.000, FIG. 11E), respectively, which revealed good discrimination. Considering clinical use in convenience, we attempted a model of concise version containing only one predictor plasma DNA that has the majority weight of predicting deterioration presented in nomogram, which achieved an AUC of 0.897 (95% CI: 0.836-0.958, FIG. 12A) with no statistical differences to the developed model (P=0.431). By setting a cut-off value of 169.3 ng/ml, plasma DNA can discriminate deterioration at a sensitivity of 85.0% and specificity of 85.9%. The decision curve analysis (DCA) of the developing subset indicated that when the threshold probability for a doctor or a patient was within a range from 0.04 to 0.92, the prediction model added more net benefit than the “treat all” or “treat none” strategies, whichever to use a two-index model or its concise one-index version (FIG. 12B).

TABLE 21
Correlation of all pairs among candidate predictors in developing subset (n = 132)

| | | Lymphocyte | Neutrophil | | Plasma | SOFA | APACHE II |
|---|---|---|---|---|---|---|---|
| | NLR* | count | count | D-dimer | DNA | score | score |
| Severe (yes vs. no) | 0.616 | −0.461 | 0.318 | 0.590 | 0.556 | 0.740 | 0.512 |
| APACHE II score | 0.518 | −0.311 | 0.350 | 0.548 | 0.406 | 0.754 | |
| SOFA score | 0.673 | −0.453 | 0.407 | 0.742 | 0.470 | | |
| Plasma DNA | 0.382 | −0.380 | NS^{†} | 0.203 | | | |
| D-dimer | 0.558 | NS^{†} | 0.582 | | | | |
| Neutrophil count | 0.503 | 0.186 | | | | | |
| Lymphocyte count | −0.729 | | | | | | |

*NLR, neutrophil to lymphocyte ratio;
^{†}NS, nonsignificant (P ≥ 0.05)

TABLE 22
Logistic regression analysis of candidate predictors in model development (n = 132)

| | Univariate | | Multivariate* | | Internal Validation^{†} | |
|---|---|---|---|---|---|---|
| | Odds Ratio (95% CI) | P value | Odds Ratio (95% CI) | P value | Odds Ratio (95% CI) | P value |
| Severity (yes vs. no) | 7.235 (2.380-21.994) | <0.001 | 0.123 (0.015-0.984) | 0.048 | | |
| APACHE II score | 1.137 (1.069-1.210) | <0.001 | | | | |
| SOFA score | 1.297 (1.150-1.464) | <0.001 | 1.186 (0.968-1.452) | 0.099 | | |
| Plasma DNA (ng/mL) | 1.012 (1.008-1.017) | <0.001 | 1.018 (1.012-1.024) | <0.001 | 1.014 (1.008-1.021) | <0.001 |
| D-dimer (ng/mL) | 1.000 (1.000-1.000) | 0.001 | 1.000 (1.000-1.000) | 0.028 | | |
| Neutrophil count (×10⁹/L) | 1.195 (1.012-1.412) | 0.036 | 1.561 (1.213-2.009) | 0.001 | 1.580 (1.216-2.053) | 0.001 |
| Lymphocyte count | 0.653 (0.364-1.169) | 0.151 | | | | |
| (×10⁹/L) | | | | | | |
| NLR^{‡} | 1.130 (1.003-1.274) | 0.045 | 0.846 (0.729-0.982) | 0.028 | | |

*Backward stepwise clustered in patient's ID;
^{†}Internal validation with bootstrapping 300 times;
^{‡}NLR: neutrophil to lymphocyte ratio.

### Case Study on Three Severe Patients

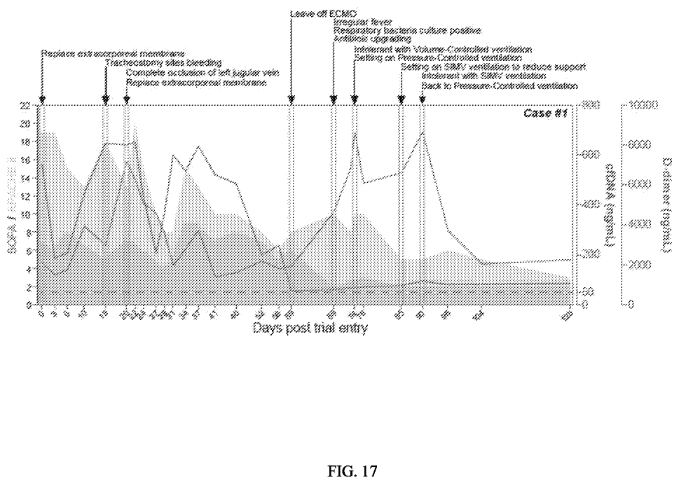

Case #1, a 61-year-old male in ICU showed that the elevated plasma DNA, except for a correlation with APACHE II score (before Day-59), coincided with irregular fever and intolerance of ventilator parameters (from Day-69 to Day-90), while severity scores and D-dimer levels were in decreasing (see FIG. 17), and PCT remained within 0.1 ng/mL. The outcome indicates that plasma DNA provided warning signs for unrecognized lung injury that other indicators such as D-dimer would not. A reasonable cause might be due to the relatively low coefficient of plasma DNA to severity scores.

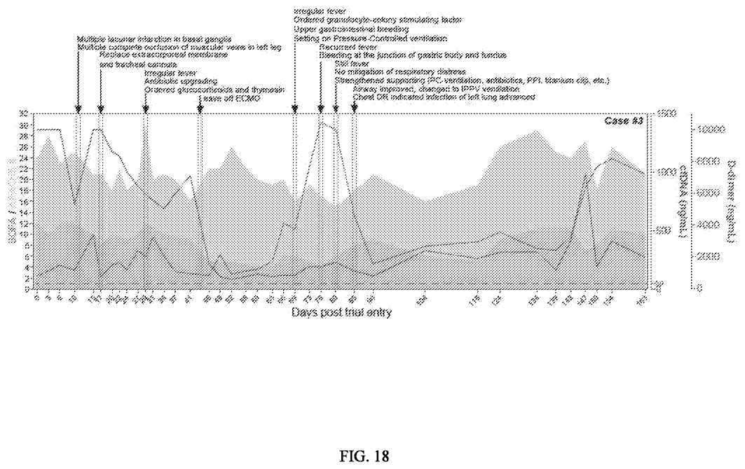

Case #3, a 68-year-old male patient in ICU had a surge of plasma DNA level with uncontrolled fever, suggested non-remission under the current treatment (see FIG. 18). The patient had recurrent fever and was found gastrorrhagia on Day-76, having a peak plasma DNA level of 1426.72 ng/mL, yet still showing stable severity scores and D-dimer level. It proposes the necessity of plasma DNA in monitoring the critical diseases.

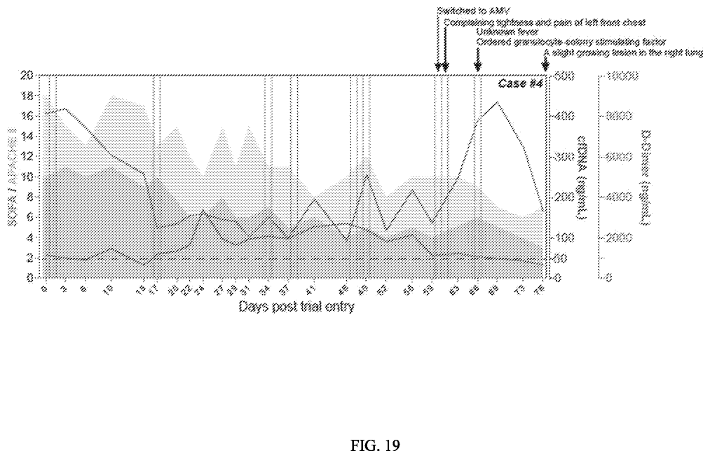

Case #4, a 66-year-old male patient in ICU, developed uncontrolled or maladaptive conditions from Day-59, which was accompanied by consistent increasing in plasma DNA but decreasing in severity scores and D-dimer. Then, the granulocyte-colony stimulating factor was provided on Day-66, and a slight growing lesion in the right lung was proved by CT ten days later (Day-76, see FIG. 19). The versatility of plasma DNA is distinct from D-dimer that primarily reflects fibrinolysis, and severity scores that synthesize systemic conditions at a high-level.

### DISCUSSION

Previous studies have found that older age, male and more comorbidities are risk factors associated with progression in COVID-19, but these personal attributes cannot reflect patient's real-time status. Most patients are remained under mild respiratory symptoms, but certain individuals might progress to severe or even critical illness, requiring specific management especially in ICU (see, e.g., Wang D et al. Clinical Characteristics of 138 Hospitalized Patients With 2019 Novel Coronavirus-Infected Pneumonia in Wuhan, China. JAMA 2020; 323:1061-9; and Wu C et al. Risk Factors Associated With Acute Respiratory Distress Syndrome and Death in Patients With Coronavirus Disease 2019 Pneumonia in Wuhan, China. JAMA Intern Med 2020; 180:934-43). However, there is no single algorithm that determines the need for invasive interventions, and clinicians must consider a variety of factors. The increasing interest in anti-IL-6 therapy is arousing the questions that whether and when elevated IL-6 represents a therapeutic target due to its double-edge of pro-inflammation and anti-inflammation (Hedrick T L et al., COVID-19: Clean up on IL-6. Am J Respir Cell Mol Biol 2020; 63:541-3). Markers that dynamically indicate severity of COVID-19 and having therapeutic relevance are still in scarcity. In this study, we examined the utility of plasma DNA level in predicting COVID-19 progression in hospitalized patients. To our knowledge, it's the first prospective study in multipoint series that showed a liquid biopsy assay, plasma DNA quantification, can be a tool for predicting disease deterioration 72 hours in advance.

A dozen years ago, for the first time, plasma DNA levels were found to increase after burn injury and were significantly correlated with the length of hospital stay (see, e.g., Chiu T W, et al., Plasma cell-free DNA as an indicator of severity of injury in burn patients. Clin Chem Lab Med 2006; 44:13-7), which suggests the potentiality of cfDNA for injury assessment. The cfDNA quantitative assays can be classified into amplification of specific target and non-amplified ones with a relatively lower specificity and sensitivity. At present, it is generally thought that circulating genomic DNA is derived mainly from apoptosis and necrosis of nucleated cells (see, e.g., Stroun M, et al., About the possible origin and mechanism of circulating DNA apoptosis and active DNA release. Clin Chim Acta 2001; 313:139-42; and Tsang J C H, Lo Y M D. Circulating nucleic acids in plasma/serum. Pathology 2007; 39:197-207) and is eliminated principally by liver (see, e.g., Celec P, Vlkova B, Laukova L, Babickova J, Boor P. Cell-free DNA: the role in pathophysiology and as a biomarker in kidney diseases. Expert Rev Mol Med 2018; 20: el). Therefore, the plasma DNA level is balanced between cfDNA release and clearance processes. Our previous report suggesting that plasma DNA could be used to monitor disease progression in patients with septic shock. However, this inference might not be drawn unanimously from other studies, as a large amount of analytic variations from sample processing to measurement are still challenging for standardization (see, e.g., Streleckiene G et al., Effects of Quantification Methods, Isolation Kits, Plasma Biobanking, and Hemolysis on Cell-Free DNA Analysis in Plasma. Biopreserv Biobank 2019; 17:553-61). The inevitable loss of cfDNA during sample preparation results in uncertain variance of analysis, which weakens the clinical applicability of cfDNA due to the incomparability between a variety of assays. By means of internal standard added into plasma and processed synchronously, and of meticulous design on consistent amplification efficiency of the standard and plasma DNA, this accurate assay, as well as the frequently used laboratory tests for infection management including blood routine and D-dimer, was evaluated for assessing COVID-19 in this study. The results from our 17 patients showed that plasma DNA levels not only can indicate the disease severity in patient triage as illustrated in FIG. 15 and perform like current criteria, including WHO severity, APACHE II and SOFA scores, to allocate limited resource efficiently, but also have a predictive value for short-term (72 hours) deterioration which overwhelmed the clinical criteria that were eliminated in the stepwise regression. We performed a thorough inspection of medical records case-by-case using a total of 174 observation visits, which revealed that most of the underlying injuries not proved by routine practice were accompanied with a sharp elevation of plasma DNA. It clearly showed, as illustrated in the three representative cases (FIGS. 17-19), that plasma DNA quantified by the accurate assay on a molecular level linking directly to tissue/organ injury can indicate disease short-term progression, which is more sensitive than clinical scores mainly based on a high-level description of symptom or physiology. Furthermore, plasma DNA, having a short half-life of about 20 minutes (see, e.g., Yu S C Y, et al. High-resolution profiling of fetal DNA clearance from maternal plasma by massively parallel sequencing. Clin Chem 2013; 59:1228-37), enabling repetitive measurements throughout the clinical monitoring, can provide dynamic feedback on disease progression, while clinical scores calculating the past cannot reflect the ongoing situations. This could be a reasonable explanation for a relative low coefficient of plasma DNA to APACHE II and SOFA scores, suggesting plasma DNA assay as a supplement to clinical scores for severity assessment, especially under emergency circumstances. Additionally, it is s a strong indicator in assessing disease progression and refining physiopathologic parameters related to patients' status that should be considered in further study.

As autopsy studies indicate a broad organotropism for the SARS-COV-2 virus beyond the lungs (see, e.g., Fox S E, et al., Pulmonary and cardiac pathology in African American patients with COVID-19: an autopsy series from New Orleans. Lancet Respir Med 2020; 8:681-6.), cfDNA methylation on tissue specificity might help elucidate COVID-19 pathogenesis. However, it remains limitations on the methylation patterning for all known cell and tissue types, and the high complexity of whole genome bisulfite sequencing precludes the clinical availability of methylation measurement. A total abundance of plasma DNA, which can be measured within two hours at low cost, appeals more feasibility in clinical scenario using a cut-off value of 95.02 ng/mL for severe discrimination. It also provides doctor a broader image of the patient's overall condition 72 hours in advance. Warning deterioration offers valuable time-window on whether to involve more frequent monitoring or interventions. The fast response of plasma DNA enables healthcare providers to manage patients more efficiently, especially under public emergencies where people sharing limited medical resources. Notably, aerosolized DNases are currently being evaluated in trials in COVID-19 (see, e.g., Weber A G, et al., Nebulized in-line endotracheal dornase alfa and albuterol administered to mechanically ventilated COVID-19 patients: a case series. Mol Med 2020; 26:91), which enables plasma DNA to be also monitored as a therapeutic target for companion diagnostics.

### Summary and Conclusions

Early recognition of COVID-19 deterioration is an urge to triage efficiently and optimize medical resources allocation. However, Markers that dynamically indicate severity of COVID-19 are still in scarcity. Here we conducted a prospective study involving hospitalized patients with confirmed COVID-19 to seek effective predictors for disease deterioration.

The consecutive hospitalized patients with confirmed COVID-19 infection were included and followed-up until discharge or 170 days of hospitalization. A series of un-overlapped 72-hour disease progressions was assessed using medical records of a 6-hour period compared against that 72 hours later. The multivariable logistic regression was used to quantify the association of predictors with the 72-hour disease deterioration. The predictive performance was assessed by nomogram and decision curve analysis.

A total of 174 un-overlapped visits from 17 patients with COVID-19 were determined 72-hour disease progression, which were correlated with plasma DNA and neutrophil count independently. A concise version of the developed model using only plasma DNA that has the majority weight for predicting deterioration in nomogram, achieved an AUC of 0.897 (95% CI: 0.836-0.958), which provides more net benefit than the “treat all” or “treat none” strategies within a range of threshold probability from 0.04 to 0.92. A thoroughly inspection in three severe cases further supported the utilities of plasma DNA to unravel deterioration.

Using a novel accurate quantification assay, plasma DNA can effectively predict COVID-19 deterioration 72 hours in advance, overwhelming APACHE II and SOFA scores, which provides a critical utility for intensive care patients.

Plasma DNA quantification provides an indispensable aid for the assessment of disease progression and the timely decision-making for patients who might require more aggressive intervention, such as intratracheal intubation or ECMO.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A double-stranded internal standard oligonucleotide for the detection of cell-free DNA in a biological sample, comprising a sequence that is at least 80% identical to the sequence of SEQ ID NO: 1.

2.-4. (canceled)

5. A method of generating an internal standard oligonucleotide, comprising
(a) providing a double-stranded oligonucleotide sequence that comprises a region of about 25-200 bp on a target human gene;
(b) inserting the oligonucleotide into a recombination vector; and
(c) digesting the recombination vector of step (b) using one or more endonucleases, thereby obtaining a linear internal standard oligonucleotide thereby generating the internal standard oligonucleotide of claim 1.

6.-15. (canceled)

16. A pharmaceutical composition comprising an effective amount of the oligonucleotide of claim 40, and a pharmaceutically acceptable carrier, diluent, or both.

17. A method for quantifying cell-free DNA in a biological sample, comprising contacting a biological sample with the oligonucleotide of claim 40.

18. The method of claim 17, further comprising detecting and quantifying a human β-actin gene in the biological sample.

19. The method of claim 18, further comprising quantifying cell-free DNA in the biological sample based on the quantification of the human β-actin gene.

20. A method for detecting or quantifying cell-free DNA in a biological sample, wherein said method comprises:
(A) incubating the biological sample with:
(1) a DNA polymerase and dNTP;
(2) a forward primer for a human β-actin gene having a nucleotide sequence consisting of SEQ ID NO: 2;
(3) a reverse primer for a human β-actin gene having a nucleotide sequence consisting of SEQ ID NO: 3; and
(4) a detectably labeled human β-actin probe, wherein the probe comprises an oligonucleotide sequence that is able to specifically hybridize to an oligonucleotide sequence of human β-actin gene; wherein the incubation is in a reaction under conditions sufficient to permit the forward and reverse primers to mediate a polymerase chain reaction amplification of a region of the human β-actin gene to thereby produce an amplified human β-actin fragment, if said human β-actin gene is present in said clinical sample; and
(B) detecting the human β-actin gene;
thereby detecting or quantifying the presence of cell-free DNA in the biological sample.

21. The method of claim 20, further comprising quantifying the human β-actin gene in the biological sample if said human β-actin gene is present in said clinical sample.

22.-23. (canceled)

24. The method of claim 20, wherein the human β-actin probe hybridizes to the amplified human β-actin fragments.

25. The method of claim 20, further comprising:
(C) adding an amount of internal standard oligonucleotides having a sequence of SEQ ID NO: 1 to the biological sample;
(D) incubating the biological sample in (C) with:
(1) a DNA polymerase and dNTP;
(2) a forward primer having a nucleotide sequence consisting of SEQ ID NO: 4;
(3) a reverse primer having a nucleotide sequence consisting of SEQ ID NO: 3; and
(4) a detectably labeled internal standard probe, wherein the probe comprises an oligonucleotide sequence that is able to specifically hybridize to the internal standard oligonucleotide;
wherein the incubation is in a reaction under conditions sufficient to permit the forward and reverse primers to mediate a polymerase chain reaction amplification of a region of the sequence of SEQ ID NO: 1 to thereby produce an amplified fragment of the region; and
(E) detecting the internal standard oligonucleotides.

26.-27. (canceled)

28. The method of claim 25, wherein the internal standard probe hybridizes to the fragments of the region of SEQ ID NO: 1.

29. (canceled)

30. The method of claim 28, wherein the DNA polymerase in (A) has a 5′→3′ exonuclease activity that hydrolyzes the hybridized human β-actin probe, wherein the DNA polymerase in (C) has a 5′→3′ exonuclease activity that hydrolyzes the hybridized internal standard probe, to thereby separate the detectable labels on the probes and cause a signal to become detected.

31.-36. (canceled)

37. The method of claim 25, wherein the quantifying of the human β-actin gene is performed based on one or more of the parameters:
(1) The starting copy number of the internal standard oligonucleotide (S₀);
(2) The amplification efficiency of the human β-actin gene (E_{T});
(3) The amplification efficiency of the internal standard oligonucleotide (E_{S});
(4) The cycle threshold for the human β-actin gene (Ct,T); and
(5) The cycle threshold for the internal standard oligonucleotide (Ct,S), wherein the quantifying of the human β-actin gene is performed according to the Formula (I)
$${T}_{0} = {S}_{0} \times \frac{{( 1 + {E}_{S} )}^{Ct , S}}{{( 1 + {E}_{T} )}^{Ct , T}} . \qquad ( I )$$

38. (canceled)

39. A kit, comprising:
(1) one or more internal standard oligonucleotide, wherein the one or more internal standard oligonucleotide comprises a sequence that is at least 90% identical to the sequence of SEQ ID NO: 1;
(2) one or more oligonucleotide, wherein the one or more oligonucleotide comprises a sequence that is at least 90% identical to the full length of an oligonucleotide sequence selected from any one of SEQ ID NOs: 2-6;
(3) a PCR buffer solution, a DNA polymerase, dNTP, and MgCl₂; and
(4) optionally instructions for performing the method of claim 17.

40. An internal standard oligonucleotide, comprising:
(a) an oligonucleotide sequence that is at least 80% identical to the corresponding region of a target human gene; and
(b) a forward primer binding site and a reverse primer binding site, wherein the length between the forward primer binding site and the reverse primer binding site is about 90 bp to about 200 bp.

41. The internal standard oligonucleotide of claim 40, wherein the reverse primer binding site is within the sequence that is at least 80% identical to the corresponding region of a target human gene.

42. The internal standard oligonucleotide of claim 40, wherein the internal standard oligonucleotide has a length of about 100 bp to about 3000 bp.

43. The internal standard oligonucleotide of claim 40, wherein the target human gene is a human housekeeping gene.

44. (canceled)

45. The method of claim 43, wherein the housekeeping gene is selected from the group consisting of: human 18S rRNA (18S ribosomal RNA), human 28S rRNA (28S ribosomal RNA), human TUBA (α-tubulin), human ACTB (β-actin), human β2M (B2-microglobulin), human ALB (albumin), human RPL32 (ribosomal protein L32), human TBP (TATA sequence binding protein), human CYCC (cyclophilin C), human EF1A (elongation factor 1α), human GAPDH (glyceraldehyde-3-phosphate dehydrogenase), human HPRT (hypoxanthine phosphoribosyl transferase), and human RPII (RNA polymerase II).

46.-58. (canceled)

59. A method of predicting the severity of an infection by SARS-COV-2, comprising:
(A) obtaining a biological sample from a subject having a SARS-COV-2 infection;
(B) quantifying cell-free DNA (cfDNA) in the biological sample using the method of claim 17; and
(C) predicting the severity based on the quantification of the cfDNA, wherein a cfDNA concentration above a cut-off value indicates deterioration of the SARS-COV-2 infection.

60.-92. (canceled)


SEQUENCE LISTING (7 sequences)

SEQ ID NO: 1 - DNA, 200 bp, synthetic construct
  source 1..200: mol_type = other DNA
ccggctcgtatgttgtgtggaattgtgagcggataacaatttcacacaggaaacagctatgaccatgattacgaattcgagctcggtacccggggatcctctagagattacgggagcggttggtggtggaaatcgtgcgtgacattaagaatcgtcgacctgcaggcatgcaagcttggcactggccgtcgttttacaac

SEQ ID NO: 2 - DNA, 24 bp, synthetic construct
  source 1..24: mol_type = other DNA
ggacctgactgactacctcatgaa

SEQ ID NO: 3 - DNA, 22 bp, synthetic construct
  source 1..22: mol_type = other DNA
cttaatgtcacgcacgatttcc

SEQ ID NO: 4 - DNA, 25 bp, synthetic construct
  source 1..25: mol_type = other DNA
aaacagctatgaccatgattacgaa

SEQ ID NO: 5 - DNA, 22 bp, synthetic construct
  source 1..22: mol_type = other DNA
caccgagcgcggctacagcttc

SEQ ID NO: 6 - DNA, 26 bp, synthetic construct
  source 1..26: mol_type = other DNA
ccaccaaccgctcccgtaatctctag

SEQ ID NO: 7 - DNA, 10454 bp, Homo sapiens
  source 1..10454: mol_type = genomic DNA
cgggaggcagaggttgcagtgagctgagattgtgccactgcagtccagcctgggcgacagagcaagactccatctcaaaacaacaacaactaaaaacgaacaacaacaacaacaaaaaactgaggccaggtgcagtggctcacacctctaatcccagcaatttgggaggctgaggtgagaggatcacttcagctcagcagttcgagaccagcccaggcaacacagggagacagaccctgttatattgcagagagaccccatctccacaaaatataaaaatattagtcagatgtggtggcatccctgccgtcccagctactcaggaggctgagacaggaggatcgcttgagcccaggaggtcaaggctgcagtgagctgtgatcatgccactgcactccagcctgggccacagagctagaccctgtctctaaaatttttagagaccttatctctaaaaataaattaaataaataaaccgggagcacctactttttctttttcttttactttttttttttttttttggagacagggtctctatcacccaggctggagtgcggtggcatgatcttggttcactgcagcctcgacccctcaggctcaggcagttctcccaccttagcctccccagtagctgggactacaggcacatgccaccatgcccggctaattttgtctttttttttttttttggtagagacagggtctcaccatgctcctcaaaactcctggactcgagagatcctcctgcctcggcctcccaaaatgctgggattacggtgtgagccgctgtgcccggctattttatttttaaatgaataaaagctggagcacccaacttttttgttgttgtttttctgagacagagttttgctcgtcacccaggctggagtgcagcggcgcaatctcggcccactgcaacctctgcctcccgggttcaagcgattctcctgcctcagcctcctgagtagctgggattacaggcacgcgccactatgcccagctaatattttgtatttttagtagagacaaggtttcatcatgtttgccaggctgatcttgaatttttgacctcaggtgatccacccgccttgacctcccaaagtgcagggattacaggcgtgagccaccacgcccagcctggagcacctaatttttaaatttaatttttcttttcttttttttttttttttgagacagggtcttcctctgtcatccaagctggaatgcagtgacttgatcatagctcactataaccttgaccttccacgctgaagcaatgctcctgccccagcctcccaattagctgggagtacaggcacgtgccacaacacccagctgattttgtagagataggatctcccgtgttgcccaagcttgtctccaactcctgggctcgagcgatcttccctcctcggcctcccaaaatgctgggattacaggtacaagccatcacaccccagtgggagagaccccacttgctgccacgtgaccatgggctgatggtgtcctctctgggcctcaggtgataaattctgaggagggaggtagggcccaggaattccgactttcaaacagcctttggactcaggccttggggacattccaggggaccctttaccagggaggggcagtgtgaggcaggaggcagggctgccgcctcagggaccctgaggctagagtccttcccaccgcatttggggacttgagcacttcaccagtcctgactcagtttcctagtctgtgaatgggggtgtgtctcctggaatgttgccccccctcattcattcattctcctctcactgagcacctaccgtgttcagggtccctgtcctcctagagctaactcaagtcttggagggaagctctgtctcagttccttcctctacaaaatggagcttgaggacgggcacggtggctcatgcctgtaatcccaggactttgggaggctgaggtgggaggatcgcttgagccccccggagctggagaccagcctgggcaacatggtgagaagccatacctctacagataatttaaaaattagccaggcttggaagtgtgtgcctatagtcccagctactcgtgaaggctttgagcccaggaggtcgaggctgcagtgagctgtgatcgcactccagcctgggcatcacagcaagaccttgtatcaaaaatagtaataataataaaaaggaggttggattccctcctggcaggatagggagggcgctgcagtgcccagggcaaggtggctgggtggttgttttgcgggagggccaaggagtggtccctgggtctgcgctgtaagagttggttgcctggaggccttgcagggtgggggtgtcaccaaagcaaaggcttggaggggaaaaaacaaaagtcccagccaggcaggtggagtccccttggctgagttcaaccgagggttctccgggggctgcgtgcgtgccccagtgacagctccgaaagctcccttacagggcaaagttcccaagcacagaagagaacctgttcacttctcccctgctcggcccgccccctggccaggcacctctacttcctcttttcctgctccgctgcttgctttctctcttcagctcctccctgcccctcaccccaggctgctcggccacctccaacctgccacctgaggacacccaggcagtcactcattcaacagcgaggagccctggggtgggtgtagtgggaaggagtgggggtgacggagaccctgggagggctcgcagcctggtggctgaggcccagttctaaatgccagctgcaagccttggtctgaggtagggaggaaggcgtggctgcagaggctaaaacgcttccccaaagaggggctttctgggatgggacttgaagggtgcataggagagcactaggaagtggccgctgcagacagagggaaccacaagccaggaggacaggccaggaatgctgcagcccggggcggggtggggctggagctcctgtctcttggccagctgaatggaggcccagtggcaacacaggtcctgcctggggatcaggtctgctctgcaccccaccttgctgcctggagccgcccacctgacaacctctcatccctgctctgcagatccggtcccatccccactgcccaccccacccccccagcactccacccagttcaacgttccacgaacccccagaaccagccctcatcaacaggcagcaagaagggccccccgcccatcgccccacaacgccagccgggtgaacgttggcaggtcctgaggcagctggcaagacgcctgcagctgaaagatacaaggccagggacaggacagtcccatccccaggaggcagggagtatacaggctggggaagtttgcccttgcgtggggtggtgatggaggaggctcagcaagtcttctggactgtgaacctgtgtctgccactgtgtgctgggtggtggtcatctttcccaccaggctgtggcctctgcaaccttcaagggaggagcaggtcccattggctgagcacagccttgtaccgtgaactggaacaagcagcctccttcctggccacaggttccatgtccttatatggactcatctttgcctattgcgacacacactcagtgaacacctactacgcgctgcaaagagccccgcaggcctgaggtgcccccacctcaccactcttcctatttttgtgtaaaaatccagcttcttgtcaccacctccaaggagggggaggaggaggaaggcaggttcctctaggctgagccgaatgcccctctgtggtcccacgccactgatcgctgcatgcccaccacctgggtacacacagtctgtgattcccggagcagaacggaccctgcccacccggtcttgtgtgctactcagtggacagacccaaggcaagaaagggtgacaaggacagggtcttcccaggctggctttgagttcctagcaccgccccgcccccaatcctctgtggcacatggagtcttggtccccagagtcccccagcggcctccagatggtctgggagggcagttcagctgtggctgcgcatagcagacatacaacggacggtgggcccagacccaggctgtgtagacccagcccccccgccccgcagtgcctaggtcacccactaacgccccaggccttgtcttggctgggcgtgactgttaccctcaaaagcaggcagctccagggtaaaaggtgccctgccctgtagagcccaccttccttcccagggctgcggctgggtaggtttgtagccttcatcacgggccacctccagccactggaccgctggcccctgccctgtcctggggagtgtggtcctgcgacttctaagtggccgcaagccacctgactcccccaacaccacactctacctctcaagcccaggtctctccctagtgacccacccagcacatttagctagctgagccccacagccagaggtcctcaggccctgctttcagggcagttgctctgaagtcggcaagggggagtgactgcctggccactccatgccctccaagagctccttctgcaggagcgtacagaacccagggccctggcacccgtgcagaccctggcccaccccacctgggcgctcagtgcccaagagatgtccacacctaggatgtcccgcggtgggtggggggcccgagagacgggcaggccgggggcaggcctggccatgcggggccgaaccgggcactgcccagcgtggggcgcgggggccacggcgcgcgcccccagcccccgggcccagcaccccaaggcggccaacgccaaaactctccctcctcctcttcctcaatctcgctctcgctctttttttttttcgcaaaaggaggggagagggggtaaaaaaatgctgcactgtgcggcgaagccggtgagtgagcggcgcggggccaatcagcgtgcgccgttccgaaagttgccttttatggctcgagcggccgcggcggcgccctataaaacccagcggcgcgacgcgccaccaccgccgagaccgcgtccgccccgcgagcacagagcctcgcctttgccgatccgccgcccgtccacacccgccgccaggtaagcccggccagccgaccggggcaggcggctcacggcccggccgcaggcggccgcggccccttcgcccgtgcagagccgccgtctgggccgcagcggggggcgcatggggggggaaccggaccgccgtggggggcgcgggagaagcccctgggcctccggagatgggggacaccccacgccagttcggaggcgcgaggccgcgctcgggaggcgcgctccgggggtgccgctctcggggcgggggcaaccggcggggtctttgtctgagccgggctcttgccaatggggatcgcagggtgggcgcggcggagcccccgccaggcccggtgggggctggggcgccattgcgcgtgcgcgctggtcctttgggcgctaactgcgtgcgcgctgggaattggcgctaattgcgcgtgcgcgctgggactcaaggcgctaactgcgcgtgcgttctggggcccggggtgccgcggcctgggctggggcgaaggcgggctcggccggaaggggtggggtcgccgcggctcccgggcgcttgcgcgcacttcctgcccgagccgctggccgcccgagggtgtggccgctgcgtgcgcgcgcgccgacccggcgctgtttgaaccgggcggaggcggggctggcgcccggttgggagggggttggggcctggcttcctgccgcgcgccgcggggacgcctccgaccagtgtttgccttttatggtaataacgcggccggcccggcttcctttgtccccaatctgggcgcgcgccggcgccccctggcggcctaaggactcggcgcgccggaagtggccagggcgggggcgacctcggctcacagcgcgcccggctattctcgcagctcaccatggatgatgatatcgccgcgctcgtcgtcgacaacggctccggcatgtgcaaggccggcttcgcgggcgacgatgccccccgggccgtcttcccctccatcgtggggcgccccaggcaccaggtaggggagctggctgggtggggcagccccgggagcgggcgggaggcaagggcgctttctctgcacaggagcctcccggtttccggggtgggggctgcgcccgtgctcagggcttcttgtcctttccttcccagggcgtgatggtgggcatgggtcagaaggattcctatgtgggcgacgaggcccagagcaagagaggcatcctcaccctgaagtaccccatcgagcacggcatcgtcaccaactgggacgacatggagaaaatctggcaccacaccttctacaatgagctgcgtgtggctcccgaggagcaccccgtgctgctgaccgaggcccccctgaaccccaaggccaaccgcgagaagatgacccaggtgagtggcccgctacctcttctggtggccgcctccctccttcctggcctcccggagctgcgccctttctcactggttctctcttctgccgttttccgtaggactctcttctctgacctgagtctcctttggaactctgcaggttctatttgctttttcccagatgagctctttttctggtgtttgtctctctgactaggtgtctaagacagtgttgtgggtgtaggtactaacactggctcgtgtgacaaggccatgaggctggtgtaaagcggccttggagtgtgtattaagtaggtgcacagtaggtctgaacagactccccatcccaagaccccagcacacttagccgtgttctttgcactttctgcatgtcccccgtctggcctggctgtccccagtggcttccccagtgtgacatggtgtatctctgccttacagatcatgtttgagaccttcaacaccccagccatgtacgttgctatccaggctgtgctatccctgtacgcctctggccgtaccactggcatcgtgatggactccggtgacggggtcacccacactgtgcccatctacgaggggtatgccctcccccatgccatcctgcgtctggacctggctggccgggacctgactgactacctcatgaagatcctcaccgagcgcggctacagcttcaccaccacggccgagcgggaaatcgtgcgtgacattaaggagaagctgtgctacgtcgccctggacttcgagcaagagatggccacggctgcttccagctcctccctggagaagagctacgagctgcctgacggccaggtcatcaccattggcaatgagcggttccgctgccctgaggcactcttccagccttccttcctgggtgagtggagactgtctcccggctctgcctgacatgagggttacccctcggggctgtgctgtggaagctaagtcctgccctcatttccctctcaggcatggagtcctgtggcatccacgaaactaccttcaactccatcatgaagtgtgacgtggacatccgcaaagacctgtacgccaacacagtgctgtctggcggcaccaccatgtaccctggcattgccgacaggatgcagaaggagatcactgccctggcacccagcacaatgaagatcaaggtgggtgtctttcctgcctgagctgacctgggcaggtcggctgtggggtcctgtggtgtgtggggagctgtcacatccagggtcctcactgcctgtccccttccctcctcagatcattgctcctcctgagcgcaagtactccgtgtggatcggcggctccatcctggcctcgctgtccaccttccagcagatgtggatcagcaagcaggagtatgacgagtccggcccctccatcgtccaccgcaaatgcttctaggcggactatgacttagttgcgttacaccctttcttgacaaaacctaacttgcgcagaaaacaagatgagattggcatggctttatttgttttttttgttttgttttggttttttttttttttttggcttgactcaggatttaaaaactggaacggtgaaggtgacagcagtcggttggagcgagcatcccccaaagttcacaatgtggccgaggactttgattgcacattgttgtttttttaatagtcattccaaatatgagatgcgttgttacaggaagtcccttgccatcctaaaagccaccccacttctctctaaggagaatggcccagtcctctcccaagtccacacaggggaggtgatagcattgctttcgtgtaaattatgtaatgcaaaatttttttaatcttcgccttaatacttttttattttgttttattttgaatgatgagccttcgtgcccccccttcccccttttttgtcccccaacttgagatgtatgaaggcttttggtctccctgggagtgggtggaggcagccagggcttacctgtacactgacttgagaccagttgaataaaagtgcacaccttaaaaatgaggccaagtgtgactttgtggtgtggctgggttgggggcagcagagggtgaaccctgcaggagggtgaaccctgcaaaagggtggggcagtgggggccaacttgtccttacccagagtgcaggtgtgtggagatccctcctgccttgacattgagcagccttagagggtgggggaggctcaggggtcaggtctctgttcctgcttattggggagttcctggcctggcccttctatgtctccccaggtaccccagtttttctgggttcacccagagtgcagatgcttgaggaggtgggaagggactatttgggggtgtctggctcaggtgccatgcctcactggggctggttggcacctgcatttcctgggagtggggctgtctcagggtagctgggcacggtgttcccttgagtgggggtgtagtgggtgttcctagctgccacgcctttgccttcacctatgggatcgtggctgtcagccttgagggtcagcctggcccaggctcccataggcttaggagaggccgcaattcctacctgttcatccagacagagggggacctggaatcaaagtcaagttggggtagggggtccatggggccatatctggcctgcagacagctctggttagctatgggctgaggtctggattctgccttgtgactggagactgggcgccatcccgtggcctctgagggcccagtttgttcgtgacccatgctgcagagggagatacccaggctcctggacctcagggcctgcctggcactgcaggccaggttctctttttatttctgagacggggtcttgtgttgcccaggctggagtgtaatggcacaaccatggctcactgcagcctcaactttgggctcaagcaattctcctgcctcagcctcccaagtagctgaaactacacaggcatgccccagcacgcccagctagttgcaggtcagattctcattgttctacagggccctgagttaaatactctcgcaagaagaaaccaagcctggcctgccttccctctgctagaagctgtgattgacaccagcagagaatgaaggaagaaaagggggtgaggatcccttgcagggatgccgtttaccccaaacaccacctggtgcatagcagttgttctggaaatgatccggtccccccaggaggcctggactctcaactgtgcacagtggcctactggcagagaggaaggtggtacccttcctcctgtgttatcttggaggtcccctgaagggctctctggggtggttctagctcttttggtctgatttctgcagtggtgagccctgggccaccttgggccacttaatcattcaacccccattcattgagtgaccactctacggtgggggaggctactgagatgacctggacaaggaccctgccctggaggagctgtcagtctggaagtgaggatttaattcatttgcccattcattcattcattcattcattcattgaggacttaattcatttgctcattcattcattcatttgctcattcatccattcattcatccatccatccatccatccaatcactatttgtgccagatagtgcgggtatagaggagaaccaaccagatgtgttccgtgtcctcaccaggcacccagtttaatggggaagacatcacaagcacgaaggcaaacaaatgagtgtacagtgacatgctgtgatcataggaagagagagcagagactttgatgaacacgagcatcgggtgatgttcattatgtcgattgtggtggtggtcttgcaggtgtagacatacttcaaaccttatctaattgggcctgctgtggtgacgcatgcttgtaatcccagcattttgggaagctgaggtgggaggatcacttgagcccaggagctcaaggctgcagtgagctatgattgcaccactgcactccagcctgggtgacagaacaagaccgtgactcttaaaaattaaaataggccaggcaggcgtggtggctcgcgcctgt